(19) United States
(12) Patent Application Publication
(10) Pub. No.: US 2008/0306102 A1
(43) Pub. Date: Dec. 11, 2008

(54) NOVEL SPIROOXYINDOLE COMPOUNDS AND DRUGS CONTAINING SAME

(75) Inventors: Hisashi NAKASHIMA, Tokyo (JP); Takaaki ARAKI, Tokyo (JP); Takahisa OGAMINO, Tokyo (JP); Yasushi KANEKO, Tokyo (JP); Kazutoyo ABE, Tokyo (JP); Kimiyuki SHIBUYA, Tokyo (JP); Tadaaki OHGIYA, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: KOWA CO., LTD., Nagoya-shi (JP)

(21) Appl. No.: 12/122,252

(22) Filed: May 16, 2008

Related U.S. Application Data
(60) Provisional application No. 60/938,763, filed on May 18, 2007.

(51) Int. Cl. A61K 31/438; C07D 471/10; A61P 3/00

(52) U.S. Cl. 514/278; 546/18

(57) ABSTRACT

A Spiro oxindole compound represented by formula (1) of the present invention or salt thereof, or their solvate shows a superior inhibitory effect of 11β-hydroxysteroid dehydrogenasel, and is useful as an agent for preventing or treating a disease that involves 11β-hydroxysteroid dehydrogenasel (in particular, diabetes, insulin resistance, diabetes complication, obesity, dyslipidemia, hypertension, fatty liver, or metabolic syndrome).

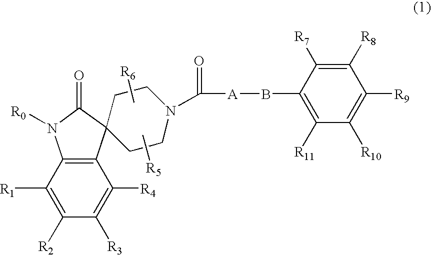

### TECHNICAL FIELD

The present invention relates to a novel Spiro oxindole compound having an 11β-hydroxysteroid dehydrogenasel-inhibitory activity and a medicine comprising the same.

### BACKGROUND ART

11β-hydroxysteroid dehydrogenase (hereinafter, abbreviated as 11β-HSD)1 is an enzyme that converts in cells an inactive form of glucocorticoid (cortisone or 11-dehydrocorticosterone) into an active form of glucocorticoid (cortisol or 11β-corticosterone), and is found to be expressed on the liver, central nerves and the like as well as subcutaneous fat and visceral fat (non-patent documents 1 and 2). Meanwhile, in cells, enzyme 11β-HSD2 is also present that converts an active form of glucocorticoid into an inactivated form. An active form of glucocorticoid is converted in cells from inactive precursor by the action of 11β-HSD1, thereby exercises its effect. Glucocorticoid has been reported to involve in adipocyte differentiation and to inhibit glycolipid metabolism that is helped by insulin (non-patent document 3). 11β-HSD1 activity and expression level in adipose tissues positively correlate with body-mass index (BMI) or insulin resistance (non-patent document 4). Further, it is reported that a transgenic mouse over-expressing 11β-HSD1 specifically in adipose tissues exhibits a phenotype comprising a combination of major factors of metabolic syndrome, such as visceral fat accumulation, insulin resistance, dyslipidemia, hypertension and fatty liver (non-patent documents 5 and 6). By contrast, it is reported that, in an 11β-HSD1 knockout mouse, an inactive form cannot be converted to an active form and as a result, the induction of the group of gluconeogenic enzymes attributable to the burden of high-fat food does not occur in the liver, which acts suppressively on hyperglycaemia due to obesity (non-patent document 7). It is also reported that decreased blood triglyceride, elevated HDL cholesterol, and improved insulin resistance were observed (non-patent document 8). From these findings, active form of glucocorticoid produced excessively by 11β-HSD1 is considered to cause the onset of a metabolic disease such as diabetes, insulin resistance, diabetes complication, obesity, dyslipidemia (hyperlipidemia), hypertension, and fatty liver, or a metabolic syndrome pathology which comprises a series of these metabolic diseases. Therefore, a selective inhibitor of 11β-HSD1 is believed to be useful for treating or preventing the above pathologies.

Heretofore, many compounds have been reported for the purpose of inhibiting 11β-HSD1 activity. The examples of reported compounds include compounds having a spiro structure (patent documents 1 to 4), adamantane derivative (patent document 5), sulfonamide derivative (patent document 6), pyrazole derivative (patent document 7), isoxazole derivative (patent document 8), triazole derivative (patent document 9), tetrazole derivative (patent document 10), pyridine derivative (patent document 11), pyrimidine derivative (patent document 12), piperidine derivative (patent document 13), pyridazine derivative (patent document 14), pyrrolidine derivative (patent document 15), thiazole derivative (patent document 16), thiophene derivative (patent document 17), lactam derivative (patent document 18) and the like. Among these, Example 56 described in the above patent document 1, (1′-{[1-(4-Chlorophenyl)cyclopropyl]carbonyl}spiro[indole-3, 4′-piperidin]-2(1H)-one) (compound A), can be given as an example of an inhibitor of 11β-HSD1 comprising spiro oxindole of the present invention. However, the compounds of general formula and examples described in this document are different from the compound of the present invention in terms of the substituent on the spiro ring (1′ position). In addition, their activity values IC₅₀ for human 11β-HSD1 are 20 μM or less, that are not potent.

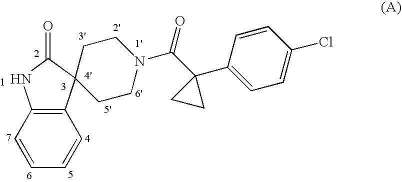

Further, patent documents 2 to 4 related to a compound having a spiro structure and other documents do not comprise a specific description that suggests the compounds of the present invention.

Patent Document 1: International publication No. WO2005/110992 pamphlet
Patent Document 2: International publication No. WO2006/040329 pamphlet
Patent Document 3: International publication No. WO2006/053024 pamphlet
Patent Document 4: International publication No. WO2006/055752 pamphlet
Patent Document 5: International publication No. WO2005/108368 pamphlet
Patent Document 6: International publication No. WO2006/134467 pamphlet
Patent Document 7: International publication No. WO2006/132436 pamphlet
Patent Document 8: International publication No. WO2006/132197 pamphlet
Patent Document 9: International publication No. WO2007/007688 pamphlet
Patent Document 10: International publication No. WO2007/029021 pamphlet
Patent Document 11: International publication No. WO2006/010546 pamphlet
Patent Document 12: International publication No. WO2006/000371 pamphlet
Patent Document 13: International publication No. WO2005/046685 pamphlet
Patent Document 14: International publication No. WO2007/003521 pamphlet
Patent Document 15: International publication No. WO2004/037251 pamphlet
Patent Document 16: International publication No. WO2006/051662 pamphlet
Patent Document 17: International publication No. WO2004/112779 pamphlet
Patent Document 18: International publication No. WO2006/049952 pamphlet
Non-patent Document 1: J. Mol. Endocrinol., 37:327-340 (2006)
Non-patent Document 2: Endcr. Rev., 25:831-866 (2004)

### Non-patent Document 3: Rinsho-i, vol. 30, No. 9, 1782-1787 (2004)

Non-patent Document 4: J. Clin. Endocrinol. Metab., 88:2738-2744 (2003)

### Non-patent Document 5: Science 294: 2166-2170 (2001)

Non-patent Document 6: J. Clin. Invest. 112:83-90 (2003)
Non-patent Document 7: Proc. Natl. Acad. Sci. USA 94:14924-14929 (1997)
Non-patent Document 8: J. Biol. Chem., 276 41293-41301 (2001)

### DISCLOSURE OF THE INVENTION

The object of the present invention is to provide a novel compound that inhibits 11β-HSD1 selectively, and is useful as a medicine.

### Means to Solve the Object

The present inventors made a keen study to find a compound that selectively inhibits 11β-HSD1. Consequently, the present inventors have found that a compound having a spiro oxindole skeleton represented by the following formula (1) is a compound that inhibits 11β-HSD1 selectively and thus completed the present invention. More specifically, the present invention relates to a spiro oxindole compound represented by the following general formula (1) or salt thereof, or their solvate:

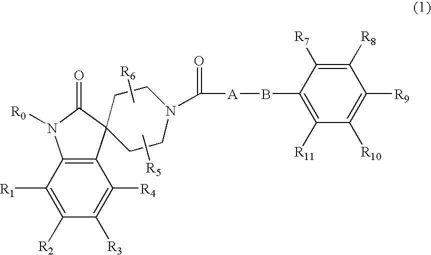

(wherein A is a —CR₁₂R₁₃—(CH₂)ⁿ— or —NR₁₄—, B is an oxygen atom, sulfur atom, —NR₁₅—, —CR₁₆R₁₇—, sulfonyl group, sulfinyl group, or —NHCO—CH₂— (here, when A is —NR₁₄—, B is —CR₁₆R₁₇—); R₀ is a hydrogen atom, alkyl group with 1 to 6 carbon atoms, alkenyl group with 2 to 6 carbon atoms, (aryl with 6 to 14 carbon atoms)-alkyl group with 1 to 6 carbon atoms, cycloalkyl group with 3 to 6 carbon atoms, (cycloalkyl with 3 to 6 carbon atoms)-alkyl group with 1 to 6 carbon atoms, or (5 to 14-membered heteroaryl)-alkyl group with 1 to 6 carbon atoms; each R₁, R₂, R₃, and R₄ are same or different and are a hydrogen atom, halogen atom, alkyl group with 1 to 6 carbon atoms, haloalkyl group with 1 to 6 carbon atoms, alkenyl group with 2 to 6 carbon atoms, (alkoxy with 1 to 6 carbon atoms)-alkyl group with 1 to 6 carbon atoms, alkynyl group with 2 to 6 carbon atoms, aryl group with 6 to 14 carbon atoms, aryloxy group with 6 to 14 carbon atoms, cycloalkyl group with 3 to 6 carbon atoms, 5 to 14-membered heteroaryl group, 5 to 14-membered heteroaryloxy group, or 5 to 7-membered heterocycloalkyl group; each R₅ and R₆ are same or different and are a hydrogen atom, alkyl group with 1 to 6 carbon atoms, haloalkyl group with 1 to 6 carbon atoms, alkenyl group with 2 to 6 carbon atoms, (alkoxy with 1 to 6 carbon atoms)-alkyl group with 1 to 6 carbon atoms, alkynyl group with 2 to 6 carbon atoms, aryl group with 6 to 14 carbon atoms, aryloxy group with 6 to 14 carbon atoms, cycloalkyl group with 3 to 6 carbon atoms, 5 to 14-membered heteroaryl group, 5 to 14-membered heteroaryloxy group, or 5 to 7-membered heterocycloalkyl group; each R₇, R₈, R₉, R₁₀, and R₁₁ are same or different and are a hydrogen atom, halogen atom, amino group, cyano group, nitro group., hydroxy group, alkoxy group with 1 to 6 carbon atoms, haloalkoxy group with 1 to 6 carbon atoms, mono- or di-alkylamino group with 1 to 6 carbon atoms, alkyl group with 1 to 6 carbon atoms, haloalkyl group with 1 to 6 carbon atoms, alkenyl group with 2 to 6 carbon atoms, (alkoxy with 1 to 6 carbon atoms)-alkyl group with 1 to 6 carbon atoms, alkynyl group with 2 to 6 carbon atoms, alkylsulfinyl group with 1 to 6 carbon atoms, alkylsulfonyl group with 1 to 6 carbon atoms, aryl group with 6 to 14 carbon atoms, aryloxy group with 6 to 14 carbon atoms, cycloalkyl group with 3 to 6 carbon atoms, 5 to 14-membered heteroaryl group, 5 to 14-membered heteroaryloxy group, or 5 to 7-membered heterocycloalkyl group, or R₇ and R₈, or R₈ and R₉ may together form a benzene ring; when they exist together, each R₁₂, R₁₃, R₁₄, R₁₅, R₁₆, and R₁₇, same or different, are a hydrogen atom, alkyl group with 1 to 6 carbon atoms, haloalkyl group with 1 to 6 carbon atoms, or alkenyl group with 2 to 6 carbon atoms; and n is an integer of 0 to 10).

The present invention also relates to a pharmaceutical composition consisting of a spiro oxindole compound represented by general formula (1) or salt thereof, or their solvate, and a pharmaceutically acceptable carrier.

Further, the present invention relates to an inhibitor of 11β-hydroxysteroid dehydrogenasel, comprising the above-mentioned spiro oxindole compound or salt thereof, or their solvate as an active ingredient.

Still further, the present invention relates to an agent for preventing and/or treating diabetes, insulin resistance, diabetes complication, obesity, dyslipidemia, hypertension, fatty liver, or metabolic syndrome, which agent comprises the above-mentioned spiro oxindole compound or salt thereof, or their solvate as an active ingredient.

Furthermore, the present invention relates to a use of the compound of the present invention for producing a formulation for inhibiting 11β-hydroxysteroid dehydrogenasel.

Furthermore, the present invention relates to a use of the compound of the present invention for producing a formulation for an agent for preventing and/or treating diabetes, insulin resistance, diabetes complication, obesity, dyslipidemia, hypertension, fatty liver, or metabolic syndrome.

Furthermore, the present invention relates to a method for inhibiting 11β-hydroxysteroid dehydrogenasel, which method comprises administering an effective amount of the compound of the present invention.

Furthermore, the present invention relates to a method for preventing and/or treating diabetes, insulin resistance, diabetes complication, obesity, dyslipidemia, hypertension, fatty liver, or metabolic syndrome, which method comprises administering an effective amount of the compound of the present invention.

### EFFECT OF THE INVENTION

The Spiro oxindole compound of the present invention shows a superior inhibitory effect of 11β-hydroxysteroid dehydrogenasel, and is useful as an agent for preventing and/or treating a disease that involves 11β-hydroxysteroid dehydrogenasel (in particular, diabetes, insulin resistance, diabetes complication, obesity, dyslipidemia, hypertension, fatty liver, or metabolic syndrome).

### BEST MODE OF CARRYING OUT THE INVENTION

The present invention will be explained in detail herein below.

A “halogen” in the present invention refers to a halogeno group, and is specifically a fluorine atom, chlorine atom, bromine atom, iodine atom or the like.

An “alkyl” in the present invention may be straight-chained or branched. Therefore, an “alkyl with 1 to 6 carbon atoms” is specifically a methyl group, ethyl group, propyl group, isopropyl group, butyl group, isobutyl group, sec-butyl group, tert-butyl group, pentyl group, isopentyl group, neo-pentyl group, hexyl group, isohexyl group or the like, or a structural isomer thereof.

A “cycloalkyl with 3 to 6 carbon atoms” in the present invention is specifically a monocyclic or polycyclic cycloalkyl group with 3 to 6 carbon atoms such as a cyclopropyl group, cyclobutyl group, cyclopentyl group, cyclohexyl group or the like, or a structural isomer thereof.

A “haloalkyl” in the present invention is an alky group substituted for same or different 1 to 3 halogen atoms. Therefore, a “haloalkyl with 1 to 6 carbon atoms” is specifically a monofluoromethyl group, difluoromethyl group, trifluoromethyl group, 2,2,2-trifluoroethyl group or the like, or a structural isomer thereof.

An “alkoxy” in the present invention may be straight-chained or branched. Therefore, an “alkoxy with 1 to 6 carbon atoms” is specifically a methyloxy group, ethyloxy group, propyloxy group, isopropyloxy group, butyloxy group, isobutyloxy group, sec-butyloxy group, tert-butyloxy group, pentyloxy group, isopentyloxy group, neo-pentyloxy group, hexyloxy group, isohexyloxy group or the like, or a structural isomer thereof.

A “haloalkoxy” in the present invention is an alkoxy group substituted for same or different 1 to 3 halogen atoms. Therefore, a “haloalkoxy with 1 to 6 carbon atoms” is specifically a monofluoromethyloxy group, difluoromethyloxy group, trifluoromethyloxy group, 2,2,2-trifluoroethyloxy group or the like, or a structural isomer thereof.

An “alkenyl” in the present invention may be straight-chained or branched. Therefore, an “alkenyl with 2 to 6 carbon atoms” is specifically a vinyl group, prop-1-en-1-yl group, allyl group, isopropenyl group, but-1-en-1-yl group, but-2-en-1-yl group, but-3-en-1-yl group, 2-methylprop-2-en-1-yl group, 1-methylprop-2-en-1-yl group, pent-1-en-1-yl group, pent-2-en-1-yl group, pent-3-en-1-yl group, pent-4-en-1-yl group, 3-methylbut-2-en-1-yl group, 3-methylbut-3-en-1-yl group, hex-1-en-1-yl group, hex-2-en-1-yl group, hex-3-en-1-yl group, hex-4-en-1-yl group, hex-5-en-1-yl group, 4-methylpent-3-en-1-yl group or the like, or a structural isomer thereof.

An “alkynyl” in the present invention may be straight-chained or branched. Therefore, an “alkynyl with 2 to 6 carbon atoms” is specifically an ethynyl group, prop-1-yn-1-yl group, prop-2-yn-1-yl group, but-1-yn-1-yl group, but-3-yn-1-yl group, 1-methylprop-2-yn-1-yl group, pent-1-yn-1-yl group, pent-4-yn-1-yl group, hex-1-yn-1-yl group, hex-5-yn-1-yl group or the like, or a structural isomer thereof.

An “aryl” in the present invention refers to a monocyclic to tricyclic aromatic hydrocarbon ring. Therefore, specific examples of an “aryl with 6 to 14 carbon atoms” include phenyl, naphthyl, azulenyl, anthryl and the like. Further, an “aryloxy with 6 to 14 carbon atoms” is specifically, for example, phenyloxy, naphthyloxy, azulenyloxy, anthryloxy or the like.

A “heteroaryl” in the present invention refers to a 5 to 14-membered (preferably, a 5 to 10-membered) monocyclic to tricyclic aromatic heterocyclic group containing 1 to 4 heteroatoms selected from an oxygen atom, sulfur atom, and nitrogen atom, or a partially saturated group thereof. Therefore, a “5 to 14-membered heteroaryl” is specifically a furyl group, thienyl group, pyrrolyl group, oxazolyl group, isoxazolyl group, dihydroisoxazolyl group, thiazolyl group, isothiazolyl group, imidazolyl group, pyrazolyl group, oxadiazolyl group, thiadiazolyl group, triazolyl group, tetrazolyl group, pyridyl group, azepinyl group, oxazepinyl group, benzofuranyl group, isobenzofuranyl group, benzothienyl group, indolyl group, isoindolyl group, indazolyl group, benzoimidazolyl group, benzoxazolyl group, benzoisoxazolyl group, benzothiazolyl group, benzoisothiazolyl group, benzoxadiazolyl group, benzothiadiazolyl group, benzotriazolyl group, chinolyl group, isochinolyl group, cinnolinyl group, quinazolinyl group, quinoxalinyl group, phthalazinyl group, naphthyridinyl group, purinyl group, pteridinyl group, carbazolyl group, carbolinyl group, acridinyl group, phenoxazinyl group, phenothiazinyl group, phenazinyl group or the like. Further, a “5 to 14-membered heteroaryloxy” is specifically a furyloxy group, thienyloxy group, pyrrolyloxy group, oxazolyloxy group, isoxazolyloxy group, dihydroisoxazolyloxy group, thiazolyloxy group, isothiazolyloxy group, imidazolyloxy group, pyrazolyloxy group, oxadiazolyloxy group, thiadiazolyloxy group, triazolyloxy group, tetrazolyloxy group, pyridyloxy group, azepinyloxy group, oxazepinyloxy group, benzofuranyloxy group, isobenzofuranyloxy group, benzothienyloxy group, indolyloxy group, isoindolyloxy group, indazolyloxy group, benzoimidazolyloxy group, benzoxazolyloxy group, benzoisoxazolyloxy group, benzothiazolyloxy group, benzoisothiazolyloxy group, benzoxadiazolyloxy group, benzothiadiazolyloxy group, benzotriazolyloxy group, chinolyloxy group, isochinolyloxy group, cinnolinyloxy group, quinazolinyloxy group, quinoxalinyloxy group, phthalazinyloxy group, naphthyridinyloxy group, purinyloxy group, pteridinyloxy group, carbazolyloxy group, carbolinyloxy group, acridinyloxy group, phenoxazinyloxy group, phenothiazinyloxy group, phenazinyloxy group or the like.

A “heterocycloalkyl” in the present invention refers to a 5 to 7-membered saturated heterocyclic group containing 1 to 4 heteroatoms selected from an oxygen atom, sulfur atom and nitrogen atom. Therefore, a “5 to 7-membered heterocycloalkyl” is specifically a pyrrolidinyl group, piperidinyl group, piperazinyl group, morpholyl group or the like.

A “monoalkylamino” in the present invention refers to a group wherein one above-mentioned alkyl group is bound to a nitrogen atom. Therefore, a “monoalkylamino with 1 to 6 carbon atoms” is specifically a methylamino group, ethylamino group, propylamino group, isopropylamino group, butylamino group, sec-butylamino group, tert-butylamino group, pentylamino group, isopentylamino group, neo-pentylamino group, hexylamino group, isohexylamino group or the like, or a structural isomer thereof.

A “dialkylamino” in the present invention refers to a group wherein two above-mentioned alkyl groups, same or different, are bound to a nitrogen atom. Therefore, a “dialkylamino with 1 to 6 carbon atoms” is specifically a dimethylamino group, methylethylamino group, diethylamino group, methylpropylamino group, ethylpropylamino group, dipropylamino group, diisopropylamino group, dibutylamino group or the like, or a structural isomer thereof.

An “(alkoxy with 1 to 6 carbon atoms)-alkyl with 1 to 6 carbon atoms” in the present invention is specifically a methyloxymethyl group, methyloxyethyl group, ethyloxymethyl group, ethyloxyethyl group or the like, or a structural isomer thereof.

An “alkylsulfonyl” in the present invention refers to a sulfonyl (SO₂) substituted for the above-mentioned alkyl. Therefore, an “alkylsulfonyl with 1 to 6 carbon atoms” is specifically a methylsulfonyl group, ethylsulfonyl group, propylsulfonyl group, butylsulfonyl group or the like, or a structural isomer thereof.

An “alkylsulfinyl” in the present invention refers to a sulfinyl (SO) substituted for the above-mentioned alkyl. Therefore, an “alkylsulfinyl with 1 to 6 carbon atoms” is specifically a methylsulfinyl group, ethylsulfinyl group, propylsulfinyl group, butylsulfinyl group or the like, or a structural isomer thereof.

An “(aryl with 6 to 14 carbon atoms)-alkyl with 1 to 6 carbon atoms” in the present invention is specifically a benzyl group, phenethyl group, 3-phenyl-n-propyl group, 4-phenyl-n-butyl group, 5-phenyl-n-pentyl group, 8-phenyl-n-hexyl group, naphthylmethyl group or the like, or a structural isomer thereof.

A “(cycloalkyl with 3 to 6 carbon atoms)-alkyl with 1 to 6 carbon atoms” in the present invention is specifically a cyclopropylmethyl group, cyclobutylmethyl group, cyclopentylmethyl group, cyclopropylethyl group, cyclobutylethyl group, cyclopentylethyl group or the like, or a structural isomer thereof.

A “(5 to 14-membered heteroaryl)-alkyl with 1 to 6 carbon atoms” in the present invention indicates a group wherein an “alkyl with 1 to 6 carbon atoms” is bound to the above-mentioned “5 to 14-membered heteroaryl”, and is specifically a 2-pyridylmethyl group, 3-pyridylmethyl group, 4-pyridylmethyl group, 2-pyridylethyl group, 3-pyridylethyl group, or 4-pyridylethyl group or the like, or a structural isomer thereof.

In general formula (1), R₀ is preferably a hydrogen atom, alkyl group with 1 to 6 carbon atoms, alkenyl group with 2 to 6 carbon atoms, cycloalkyl group with 3 to 6 carbon atoms, or (cycloalkyl with 3 to 6 carbon atoms)-alkyl group with 1 to 6 carbon atoms.

In general formula (1), an alkyl group with 1 to 6 carbon atoms of R₀ is more preferably a methyl group, ethyl group, propyl group, or isopropyl group.

In general formula (1), a cycloalkyl group with 3 to 6 carbon atoms of R₀ is more preferably a cyclopropyl group.

In general formula (1), a (cycloalkyl with 3 to 6 carbon atoms)-alkyl group with 1 to 6 carbon atoms of R₀ is more preferably a cyclopropylmethyl group.

In general formula (1), preferred R₁, R₂, R₃, and R₄ are a hydrogen atom.

In general formula (1), preferred R₅ and R₆ are a hydrogen atom.

Preferred examples of R₇, R₈, R₉, R₁₀ and R₁₁ in general formula (1) include a hydrogen atom, halogen atom, cyano group, nitro group, alkyl group with 1 to 6 carbon atoms, haloalkyl group with 1 to 6 carbon atoms, alkoxy group with 1 to 6 carbon atoms, or aryl group with 6 to 14 carbon atoms, and more preferred examples include a hydrogen atom, halogen atom, nitro group, alkyl group with 1 to 6 carbon atoms, or haloalkyl group with 1 to 6 carbon atoms.

In general formula (1), a halogen atom of R₇, R₈, R₉, R₁₀ and R₁₁ is more preferably a fluorine atom, chlorine atom, bromine atom, or iodine atom.

In general formula (1), an alkyl group with 1 to 6 carbon atoms of R₇, R₈, R₉, R₁₀ and R₁₁ is more preferably a methyl group, ethyl group, propyl group, butyl group, or tert-butyl group, and even more preferably a methyl group or tert-butyl group.

In general formula (1), a haloalkyl group with 1 to 6 carbon atoms of R₇, R₈, R₉, R₁₀ and R₁₁ is more preferably a trifluoromethyl group.

In general formula (1), an alkoxy group with 1 to 6 carbon atoms of R₇, R₈, R₉, R₁₀ and R₁₁ is more preferably a methoxy group.

In general formula (1), an aryl group with 6 to 14 carbon atoms of R₇, R₈, R₉, R₁₀ and R₁₁ is more preferably a phenyl group.

In general formula (1), R₁₂, R₁₃, R₁₄, R₁₅, R₁₆, and R₁₇ are preferably a hydrogen atom or alkyl group with 1 to 6 carbon atoms, and more preferably a hydrogen atom.

In general formula (1), A is more preferably a —CR₁₂R₁₃—(CH₂)ₙ—, wherein n is an integer of 0 to 10, more preferably the integer 0.

In general formula (1), B is preferably an oxygen atom or sulfur atom, and more preferably an oxygen atom.

When an asymmetric carbon atom is present in the Spiro oxindole compound shown by general formula (1) of the present invention, there exists an optical isomer, and the present invention encompasses those optical isomers or any mixtures comprising racemate and the like.

The present invention also encompasses various hydrates or solvates of the Spiro oxindole compound shown by general formula (1) or pharmaceutically-acceptable acid-addition salt thereof, and a crystal polymorphic substance of the same.

Examples of pharmaceutically acceptable salt of the Spiro oxindole compound shown by general formula (1) specifically include acid addition salt and the like treated with an inorganic acid (for example, a hydrochloric acid, hydrobromic acid, hydroiodic acid, sulfuric acid, nitric acid, phosphoric acid and the like) or an organic acid (for example, a formic acid, acetic acid, propionic acid, oxalic acid, malonic acid, succinic acid, fumaric acid, maleic acid, lactic acid, malic acid, tartaric acid, citric acid, methanesulfonic acid, ethanesulfonic acid, p-toluenesulfonic acid, asparaginic acid, glutamic acid and the like).

Examples of solvates of the Spiro oxindole compound shown by general formula (1) or pharmaceutically-acceptable salt thereof include hydrates or various solvates (for example, a solvate with alcohol such as ethanol).

Among the Spiro oxindole compounds shown by general formula (1), examples of a compound or salt, or their solvate having a particularly preferred combination include those having a combination wherein R₀ is a hydrogen atom or alkyl group with 1 to 6 carbon atoms; R₁, R₂, R₃, R₄, R₅, and R₆ are a hydrogen atom; R₇, R₈, R₉, R₁₀, and R₁₁ are same or different and are a hydrogen atom, halogen atom, nitro group, alkyl group with 1 to 6 carbon atoms, or haloalkyl group with 1 to 6 carbon atoms; and R₁₂, R₁₃, R₁₄, R₁₅, R₁₆, and R₁₇ are represented by a hydrogen atom.

As a compound of the present invention, the following compounds, pharmaceutically acceptable salts thereof, and their solvates are particularly preferred:
1′-{2-(2-trifluoromethylphenoxy)acetyl}spiro(indole-3,4′-piperidine)-2(1H)-one,
1′-{2-(2-methylphenoxy)acetyl}spiro(indole-3,4′-piperidine)-2(1H)-one,
1′-{2-(2-iodophenoxy)acetyl}spiro(indole-3,4′-piperidine)-2(1H)-one,
1′-{2-(2-nitrophenoxy)acetyl}spiro(indole-3,4-piperidine)-2(1H)-one,
1′-{2-(2,3-dichlorophenoxy)acetyl}spiro(indole-3,4′-piperidine)-2(1H)-one,
1′-{2-(2-bromophenoxy)acetyl}spiro(indole-3,4′-piperidine)-2(1H)-one,
1′-{2-(2-t-butylphenoxy)acetyl}spiro(indole-3,4′-piperidine)-2(1H)-one,
1′-{2-(2-trifluoromethylphenylthio)acetyl}spiro(indole-3,4′-piperidine)-2(1H)-one,
1-methyl-1′-{2-(2-trifluoromethylphenoxy)acetyl}spiro(indol e-3,4′-piperidine)-2-one,
1-ethyl-1′-{2-(2-trifluoromethylphenoxy)acetyl}spiro(indole-3,4′-piperidine)-2-one,
1-propyl-1′-{2-(2-trifluoromethylphenoxy)acetyl}spiro(indol e-3,4′-piperidine)-2-one, and
1-cyclopropyl-1′-{2-(2-trifluoromethylphenoxy)acetyl}spiro(indole-3,4′-piperidine)-2-one.

### (Method for Producing Compounds Represented by General Formula (I) or Solvates Thereof)

I. Method for Producing. Compounds Represented by (I) or Solvates Thereof

Reacting cyclic amines shown by general formula (II) with carboxylic acids shown by general formula (III) or reactive derivatives thereof yields amide derivatives shown by general formula (IV). Alkylating benzene derivatives shown by general formula (V) to the obtained compounds shown by general formula (IV) in the presence of a base produces spiro oxindole compounds (I) of interest. The reaction path is shown by the following chemical formula.

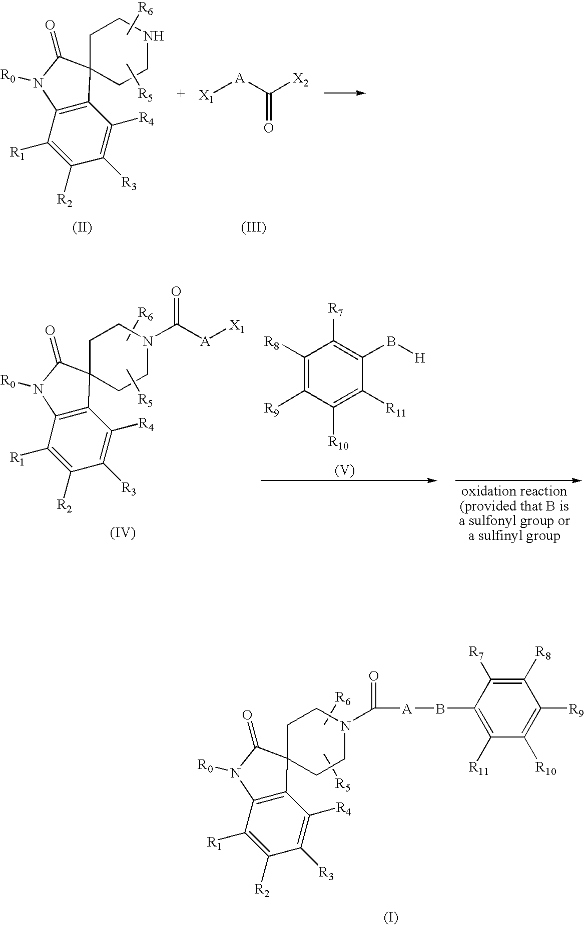

(wherein R₀, R₁, R₂, R₃, R₄, R₅, R₆, R₇, R₈, R₉, R₁₀, R₁₁, A, and B show the same things as they show in the above general formula (I), X₁ shows a hydroxy group, halogen atom, alkylsulfonyloxy group, haloalkylsulfonyloxy group, or arylsulfonyloxy group, and X₂ shows a halogen atom, hydroxy group, or aryloxy group).

A reaction of an acyl halide compound (III) wherein X₂ is a halogen atom, with an amine compound (II) can be conducted in a solvent in the presence or absence of a base. A solvent is not particularly limited, and for example, the followings can be used independently or in combination: 1,2-dichloroethane, chloroform, methylene chloride, ethyl acetate, isopropyl acetate, toluene, benzene, tetrahydrofuran, dioxane, acetonitrile, propionitrile, N,N-dimethylformamide and the like. A base is not particularly limited, and for example, the followings can be used: organic bases such as pyridine, N,N-dimethylaminopyridine (DMAP), collidine, lutidine, 1,8-diazabicyclo[5.4.0]undecene (DBU), 1,5-diazabicyclo[4,3,0]nonene (DBN), 1,4-diazabicyclo[2.2.2]octene (DABCO), triethylamine, 2,6-di-t-butylpyridine, N,N-diisopropylethylamine, N,N-diisopropylpentylamine, N-methylmorpholine, and trimethylamine; alkali metal hydrides such as lithium hydride, sodium hydride, and potassium hydride; alkali metal hydroxides such as lithium hydroxide, sodium hydroxide, and potassium hydroxide; alkali metal carbonates such as lithium carbonate, sodium carbonate, potassium carbonate, and cesium carbonate; bicarbonate metals such as sodium hydrogen carbonate and potassium hydrogen carbonate; lithium diisopropylamide, sodium diisopropylamide, potassium diisopropylamide, lithium hexamethyldisilazide, sodium hexamethyldisilazide, potassium hexamethyldisilazide, sodium t-butoxide, potassium t-butoxide, n-butyllithium, s-butyllithium, t-butyllithium and the like. The reaction condition varies depending on the materials used, but generally, an amide compound (IV) is obtained by conducting the reaction at −20 to 100° C., preferably at 0 to 30° C. for 5 minutes to 1 day, preferably for 2 to 12 hours.

A reaction of a carboxylic compound (III) wherein X₂ is a hydroxy group, with a compound (II) can be conducted in a solvent using a condensation agent in the presence or absence of a base, and in the presence or absence of a condensation accelerator. A solvent is not particularly limited, and for example, the followings can be used: 1,2-dichloroethane, chloroform, methylene chloride, ethyl acetate, isopropyl acetate, toluene, benzene, tetrahydrofuran, dioxane, acetonitrile, propionitrile, N,N-dimethylformamide, N-methylpyrrolidone and the like. A base is not particularly limited, and for example, the followings can be used: organic bases such as pyridine, DMAP, collidine, lutidine, DBU, DBN, DABCO, triethylamine, diisopropylethylamine, diisopropylpentylamine, trimethylamine and the like; alkali metal hydrides such as lithium hydride, sodium hydride, and potassium hydride; alkali metal hydroxides such as lithium hydroxide, sodium hydroxide, and potassium hydroxide; alkali metal carbonates such as lithium carbonate, sodium carbonate, potassium carbonate, and cesium carbonate; and bicarbonates such as sodium hydrogen carbonate and potassium hydrogen carbonate. A condensation accelerator is not particularly limited, and the followings can be used: DMAP, 1-hydroxy-7-azobenzotriazole (HOAt), 1-hydroxybenzotriazole (HOBT), 3-hydroxy-3,4-dihydro-4-oxo-1,2,3-benzotriazole (HODhbt), N-hydroxy-5-norbornene-2,3-dicarboxylmide (HONB), pentafluorophenol (HOPfp), N-hydroxyphthalimide (HOPht), N-hydroxysuccinimide (HOSu) and the like. A condensation agent is not particularly limited, and the followings can be used: N,N′-dicyclohexylcarbodiimide (DCC), N,N′-diisopropylcarbodiimide (DIPCI), 1-ethyl-3-(3-dimethylaminopropyl)carbodiimide (WSCI), 1-ethyl-3-(3-dimethylaminopropyl)carbodiimide hydrochloride (WSC.HCl), diethyl cyanophosphate (DEPC), benzotriazole-1-yl-oxy-tris(dimethylamino)phosphoniumhexa fluorophosphate (BOP), benzotriazole-1-yl-oxy-tris(pyrrolidinylamino)phosphoniumhe xa fluorophosphate (PyBOP), 2-(1H-benzotriazole-1-yl)-1,1,3,3-tetramethyluronium tetrafluoroborate (TBTU) and the like. The reaction condition varies depending on the materials used, but generally, an amide compound (IV) is obtained by conducting the reaction at −20 to 100° C., preferably at 0 to 30° C. for 5 minutes to 1 day, preferably for 2 to 12 hours.

When X₁ of the obtained amide compound (IV) is a leaving group (a halogen atom, alkylsulfonyloxy group, haloalkylsulfonyloxy group, or arylsulfonyloxy group), the reaction of a compound (IV) with a benzene derivative (V) can be conducted in a solvent in the presence of a base. A solvent is not particularly limited, and for example, the followings can be used independently or in combination: tetrahydrofuran, toluene, dioxane, N,N-dimethylformamide, N-methylpyrrolidone, methylene chloride, acetonitrile, propionitrile and the like. A base is not particularly limited, and for example, the followings can be used: alkali metal hydrides such as lithium hydride, sodium hydride, and potassium hydride; alkali metals such as lithium, sodium, and potassium; alkali metal hydroxides such as lithium hydroxide, sodium hydroxide, and potassium hydroxide; alkali metal carbonates such as lithium carbonate, sodium carbonate, potassium carbonate, and cesium carbonate; DABCO, lithium diisopropylamide, sodium diisopropylamide, potassium diisopropylamide, lithium hexamethyldisilazide, sodium hexamethyldisilazide, potassium hexamethyldisilazide, sodium t-butoxide, potassium t-butoxide, n-butyllithium, s-butyllithium, t-butyllithium and the like. The reaction condition varies depending on the materials used, but generally, a spiro oxindole compound (I) of interest is obtained by conducting the reaction at −20 to 150° C., preferably at 15 to 80° C. for 5 minutes to 1 day, preferably for 5 to 12 hours.

When X₁ of the obtained amide compound (IV) is a hydroxy group, the compound (IV) and a benzene derivative (V) can be subjected to the Mitsunobu reaction. Examples of a phosphorous compound used in this process include a phosphine reagent; a phosphorous reagent consisting of the phosphine reagent and an azo reagent or an ethylenedicarboxylic acid reagent such as dimethyl maleate and N,N,N′,N′-tetramethylfumaramide; and a phosphonium ylide reagent and the like, used in the Mitsunobu reaction. Examples of a preferred embodiment of this process include 1) a method of reacting a benzene derivative (V) or salt thereof in the presence of a phosphine reagent and azo reagent or an ethylenedicarboxylic acid reagent such as dimethyl maleate, N,N,N′,N′-tetramethylfumaramide and the like (the first method), and 2) a method of reacting a benzene derivative (V) or salt thereof in the presence of a phosphonium ylide reagent (the second method).

The first method can be conducted by dissolving an amide compound (IV), benzene derivative (V) or salt thereof, and a phosphine reagent in a reaction solvent, and adding thereto an azo reagent or ethylenedicarboxylic acid reagent, and performing a reaction under an argon or nitrogen atmosphere at 0° C. to 100° C., preferably at room temperature to 80° C. for 2 hours to 1 day. As a solvent to be used in this reaction, the followings can be used: N,N-dimethylformamide, tetrahydrofuran, dioxane, acetonitrile, nitromethane, acetone, ethyl acetate, benzene, chlorobenzene, toluene, chloroform, methylene chloride and the like. Among these, N,N-dimethylformamide, tetrahydrofuran, dioxane, and acetonitrile are preferred, and N,N-dimethylformamide and tetrahydrofuran are particularly preferred. Examples of a phosphine reagent include, for example, trialkylphosphines such as trimethylphosphine, triethylphosphine, tripropylphosphine, triisopropylphosphine, tributylphosphine, triisobutylphosphine, and tricyclohexylphosphine; and arylphosphines such as triphenylphosphine and diphenylphosphino polystyrene. Among these, trimethylphosphine, tributylphosphine, and triphenylphosphine are preferred. Examples of an azo reagent include, for example, diethyl azodicarboxylate (DEAD), diisopropyl azodicarboxylate, 1,1′-azobis(N,N-dimethylformamide) (TMAD), 1,1′-(azodicarbonyl)dipiperidine (ADDP), 1,1′-azobis(N,N-diisopropylformamide) (TIPA), 1,6-dimethyl-1,5,7-hexahydro-1,4,6,7-tetrazocine-2,5-dion (DHTD) and the like, and diethyl azodicarboxylate is particularly preferred.

The second method can be conducted by dissolving in a reaction solvent an amide compound (IV), benzene derivative (V) or salt thereof, and a phosphonium ylide reagent, and performing a reaction under an argon or nitrogen atmosphere at room temperature to 120° C., preferably at 80° C. to 100° C. for 2 hours to 12 hours. Examples of a phosphonium ylide reagent used in the reaction include alkanoylmethylene trialkylphosphorane, alkanoylmethylene triarylphosphorane, alkoxycarbonylmethylene trialkylphosphorane, alkoxycarbonylmethylene triarylphosphorane, cyanomethylene trialkylphosphorane, cyanomethylene triarylphosphorane and the like. Here, examples of trialkyl include trimethyl, triethyl, tripropyl, triisopropyl, tributyl, triisobutyl, tricyclohexyl and the like, and examples of triaryl include triphenyl, diphenyl polystyrene and the like. Further, the reaction may be conducted using a method comprising allowing a phosphonium halide reagent to act on an amide compound (IV), benzene derivative (V) or salt thereof in the presence of a base to produce a phosphonium ylide reagent in the reaction system. Examples of a phosphonium halide reagent used in this case include, for example, (cyanomethyl) trialkylphosphonium halide, (cyanomethyl)triarylphosphonium halide, (alkylcarbonylmethyl)trialkylphosphonium halide, (alkylcarbonylmethyl)triarylphosphonium halide, (alkoxycarbonylmethyl)trialkylphosphonium halide, (alkoxycarbonylmethyl)triarylphosphonium halide and the like.

A compound wherein B of formula (1) is a sulfonyl group or sulfinyl group can be obtained by allowing a reaction product from amide compound (IV) and thiophenols (V) to go through a common sulfur-atom oxidation reaction. As an oxidant, for example, 3-chloroperbenzoic acid, peracetic acid, sodium periodate or the like can be used. Further, hydrogen peroxide can also be used in the presence or absence of tantalum pentachloride. The reaction condition varies depending on the material used, but generally, a spiro oxindole compound (I) of interest is obtained by conducting the reaction at −20 to 50° C., preferably at 0 to 30° C. for 5 minutes to 1 day, preferably for 1 to 12 hours.

Further, a compound shown by formula (1) can also be produced according to the reaction formula below.

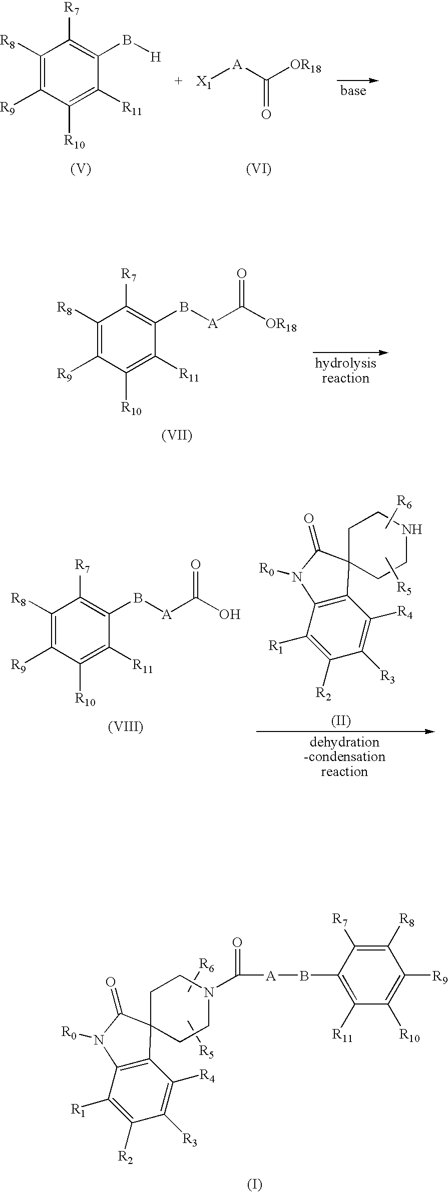

(wherein R₀, R₁, R₂, R₃, R₄, R₅, R₆, R₇, R₈, R₉, R₁₀, R₁₁, A, and B show the same things as they show in the above-mentioned general formula (1), R₁₈ shows a lower-alkyl group, and X₁ shows a halogen atom, alkylsulfonyloxy group, haloalkylsulfonyloxy group, or arylsulfonyloxy group).

A reaction of a benzene derivative (V) with an ester derivative (VI) having a leaving group can be conducted in a solvent in the presence of a base. A solvent is not particularly limited, and for example, the followings can be used independently or in combination: tetrahydrofuran, toluene, dioxane, N,N-dimethylformamide, N-methylpyrrolidone, methylene chloride, acetonitrile, propionitrile and the like. A base is not particularly limited, and for example, the followings can be used: alkali metal hydrides such as lithium hydride, sodium hydride, and potassium hydride; alkali metals such as lithium, sodium, and potassium; alkali metal hydroxides such as lithium hydroxide, sodium hydroxide, and potassium hydroxide; alkali metal carbonates such as lithium carbonate, sodium carbonate, potassium carbonate, and cesium carbonate; DABCO, lithium diisopropylamide, sodium diisopropylamide, potassium diisopropylamide, lithium hexamethyldisilazide, sodium hexamethyldisilazide, potassium hexamethyldisilazide, sodium t-butoxide, potassium t-butoxide, n-butyllithium, s-butyllithium, t-butyllithium and the like. The reaction condition varies depending on the materials used, but generally, a substance of interest (VII) is obtained by conducting the reaction at −20 to 150° C., preferably at 15 to 80° C. for 5 minutes to 1 day, preferably for 5 hours to 15 hours.

The obtained ester derivative (VII) is subjected to a usual hydrolysis reaction to yield a carboxylic acid derivative (VIII). The reaction can be conducted in a solvent in the presence of a base or acid. A solvent is not particularly limited, and for example, the followings can be used independently or in combination: tetrahydrofuran, dioxane, methanol, ethanol, water and the like. A base is not particularly limited, and for example, the followings can be used: alkali metal hydroxides such as sodium hydroxide and potassium hydroxide; alkali metal carbonates such as lithium carbonate, sodium carbonate, potassium carbonate, and cesium carbonate; potassium trimethylsilanolate and the like. An acid is not particularly limited, and the followings can be used: hydrochloric acid, acetic acid, trifluoroacetic acid, boron tribromide, aluminium chloride and the like. The reaction condition varies depending on the materials used, but generally, a carboxylic acid derivative (VIII) is obtained by conducting the reaction at −20 to 100° C., preferably at 15 to 50° C. for 5 minutes to 1 day, preferably for 1 hour to 12 hours.

A condensation reaction of the obtained carboxylic acid derivative (VIII) with a compound (II) can be conducted in a solvent using a condensation agent in the presence or absence of a base, and in the presence or absence of a condensation accelerator. A solvent is not particularly limited, and for example, the followings can be used: 1,2-dichloroethane, chloroform, methylene chloride, ethyl acetate, isopropyl acetate, toluene, benzene, tetrahydrofuran, dioxane, acetonitrile, propionitrile, N,N-dimethylformamide, N-methylpyrrolidone and the like. A base is not particularly limited, and for example, the followings can be used: organic bases such as pyridine, DMAP, collidine, lutidine, DBU, DBN, DABCO, triethylamine, diisopropylethylamine, diisopropylpentylamine, and trimethylamine; alkali metal hydrides such as lithium hydride, sodium hydride, and potassium hydride; alkali metal hydroxides such as lithium hydroxide, sodium hydroxide, and potassium hydroxide; alkali metal carbonates such as lithium carbonate, sodium carbonate, potassium carbonate, and cesium carbonate; and bicarbonates such as sodium hydrogen carbonate and potassium hydrogen carbonate. A condensation accelerator is not particularly limited, and DMAP, HOAt, HOBt, HODhbt, HONB, HOPfp, HOPht, HOSu and the like can be used. A condensation agent is not particularly limited, and DCC, DIPCI, WSCI, WSC.HCl, DEPC, BOP, PyBOP, TBTU and the like can be used. The reaction condition varies depending on the materials used, but generally, a spiro oxindole compound (I) of interest is obtained by conducting the reaction at −20 to 100° C., preferably at 0 to 30° C. for 5 minutes to 1 day, preferably for 2 to 12 hours.

Further, a compound shown by formula (1) can also be produced according to the reaction formula below.

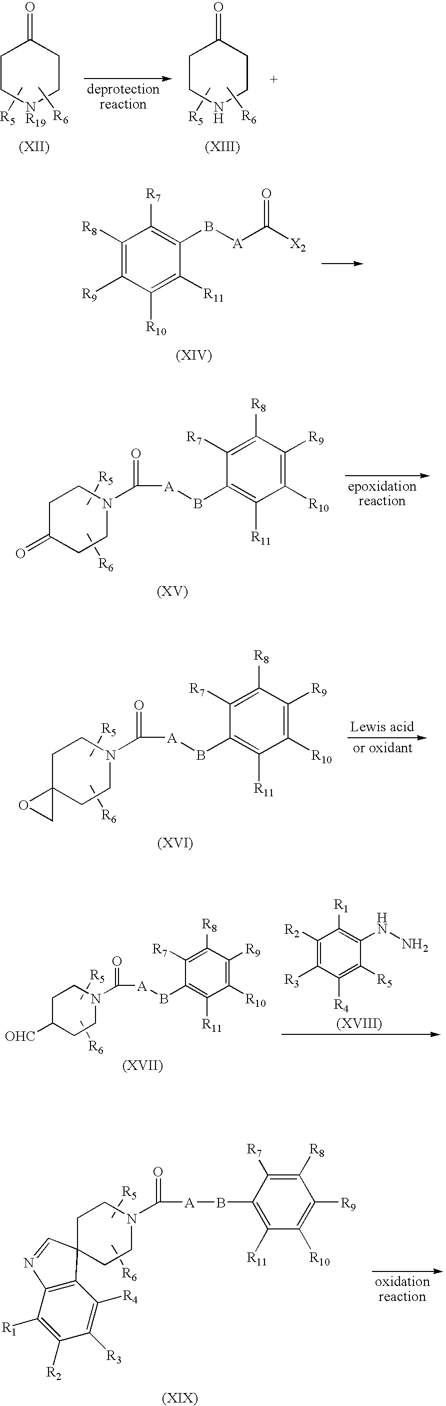

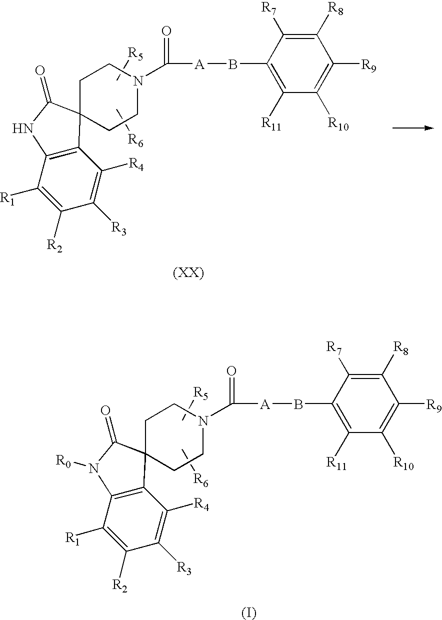

(wherein R₀, R₁, R₂, R₃, R₄, R₅, R₆, R₇, R₈, R₉, R₁₀, R₁₁, A, and B show the same things as they show in the above-mentioned general formula (1), R₁₉ shows a protecting group, and X₂ shows a halogen atom, hydroxy group or aryloxy group).

The above compound (XII) can be synthesized by a known method. For example, a reference can be made to methods described in a patent document, International Publication No. WO1997/036871 Pamphlet and a non-patent document, Bull. Chem. Soc. Jpn., Vol. 34:418-422 (1958).

A deprotection of protecting group R₁₉ of the above compound (XII) is not particularly limited, which can be conducted with reference to a commonly used method (Protective Groups in Organic Synthesis Third Edition, John Wiley & Sons, Inc.) as a deprotection condition of the protecting group. A protecting group is not particularly limited, and for example, the followings can be used: a benzyl group, 9-fluorenylmethoxycarbonyl group (Fmoc group), 2,2,2-trichloroethoxycarbonyl group (Troc group), 2-(trimethylsilyl)ethoxycarbonyl group (Teoc group), t-butoxycarbonyl group (Boc group), allyloxycarbonyl group (Alloc group), vinyloxycarbonyl group, benzyloxycarbonyl group (Cbz group), p-methoxybenzyloxydarbonyl group, p-nitrobenzyloxycarbonyl group, allyl group, 2-(trimethylsilyl)ethoxymethyl group (SEM group), 4-methoxybenzyl group, triphenylmethyl group, benzenesulfonyl group, and o-nitrobenzenesulfonyl group. In particular, a benzyl group, Fmoc group, Boc group, and Cbz group are preferred.

A reaction of an acyl halide compound (XIV) wherein X₂ is a halogen atom with a compound (XIII) can be conducted in a solvent in the presence or absence of a base. A solvent is not particularly limited, and for example, the followings can be used independently or in combination: 1,2-dichloroethane, chloroform, methylene chloride, ethyl acetate, isopropyl acetate, toluene, benzene, tetrahydrofuran, dioxane, acetonitrile, propionitrile, N,N-dimethylformamide and the like. A base is not particularly limited, and for example, the followings can be used: organic bases such as pyridine, N,N-dimethylaminopyridine (DMAP), collidine, lutidine, 1,8-diazabicyclo[5.4.0]undecene (DBU), 1,5-diazabicyclo[4,3,0]nonene (DBN), 1,4-diazabicyclo[2.2.2]octene (DABCO), triethylamine, 2,6-di-t-butylpyridine, N,N-diisopropylethylamine, N,N-diisopropylpentylamine, N-methylmorpholine, and trimethylamine; alkali metal hydrides such as lithium hydride, sodium hydride, and potassium hydride; alkali metal hydroxides such as lithium hydroxide, sodium hydroxide, and potassium hydroxide; alkali metal carbonates such as lithium carbonate, sodium carbonate, potassium carbonate, and cesium carbonate; bicarbonate metals such as sodium hydrogen carbonate and potassium hydrogen carbonate; lithium diisopropylamide, sodium diisopropylamide, potassium diisopropylamide, lithium hexamethyldisilazide, sodium hexamethyldisilazide, potassium hexamethyldisilazide, sodium t-butoxide, potassium t-butoxide, n-butyllithium, s-butyllithium, t-butyllithium and the like. The reaction condition varies depending on the materials used, but generally, an amide compound (XV) is obtained by conducting the reaction at −20 to 100° C., preferably at 0 to 30° C. for 5 minutes to 1 day, preferably for 2 to 12 hours.

A reaction of a carboxylic acid compound (XIV) wherein X₂ is a hydroxy group with a compound (XIII) can be conducted in a solvent using a condensation agent in the presence or absence of a base, and in the presence or absence of a condensation accelerator. A solvent is not particularly limited, and for example, the followings can be used: 1,2-dichloroethane, chloroform, methylene chloride, ethyl acetate, isopropyl acetate, toluene, benzene., tetrahydrofuran, dioxane, acetonitrile, propionitrile, N,N-dimethylformamide, N-methylpyrrolidone and the like. A base is not particularly limited, and for example, the followings can be used: organic bases such as pyridine, DMAP, collidine, lutidine, DBU, DBN, DABCO, triethylamine, diisopropylethylamine, diisopropylpentylamine, and trimethylamine; alkali metal hydrides such as lithium hydride, sodium hydride, and potassium hydride; alkali metal hydroxides such as lithium hydroxide, sodium hydroxide, and potassium hydroxide; alkali metal carbonates such as lithium carbonate, sodium carbonate, potassium carbonate, and cesium carbonate; and bicarbonates such as sodium hydrogen carbonate and potassium hydrogen carbonate. A condensation accelerator is not particularly limited, and the followings can be used: DMAP, 1-hydroxy-7-azobenzotriazole (HOAt), 1-hydroxybenzotriazole (HOBT), 3-hydroxy-3,4-dihydro-4-oxo-1,2,3-benzotriazole (HODhbt), N-hydroxy-5-norbornene-2,3-dicarboxylmide (HONB), pentafluorophenol (HOPfp), N-hydroxyphthalimide (HOPht), N-hydroxysuccinimide (HOSu) and the like. A condensation, agent is not particularly limited, and the followings can be used: N,N′-dicyclohexylcarbodiimide (DCC), N,N′-diisopropylcarbodiimide (DIPCI), 1-ethyl-3-(3-dimethylaminopropyl)carbodiimide (WSCI), 1-ethyl-3-(3-dimethylaminopropyl)carbodiimide hydrochloride (WSC.HCl), diethyl cyanophosphate (DEPC), benzotriazole-1-yl-oxy-tris(dimethylamino)phosphonium hexafluorophosphate (BOP), P benzotriazole-1-yl-oxy-tris(pyrrolidinylamino)phosphonium hexafluorophosphate (PyBOP), 2-(1H-benzotriazole-1-yl)-1,1,3,3-tetramethyluronium tetrafluoroborate (TBTU) and the like. The reaction condition varies depending on the materials used, but generally, an amide compound (XV) is obtained by conducting the reaction at −20 to 100° C., preferably at 0 to 30° C. for 5 minutes to 1 day, preferably for 2 to 12 hours.

An epoxidation reaction of a compound (XV) can be conducted in a solvent in the presence of a base using trimethylsulfoxonium iodide. A solvent is not particularly limited, and for example, the followings can be used independently or in combination: dimethylsulfoxide (DMSO), N,N-dimethylformamide, N-methylpyrrolidone, tetrahydrofuran, toluene, dioxane and the like. A base is not particularly limited, and for example, the followings can be used: lithium hydride, sodium hydride, potassium hydride, lithium diisopropylamide, sodium diisopropylamide, potassium diisopropylamide, lithium hexamethyldisilazide, sodium hexamethyldisilazide, potassium hexamethyldisilazide, sodium t-butoxide, potassium t-butoxide, n-butyllithium, s-butyllithium, t-butyllithium and the like. The reaction condition varies depending on the materials used, but generally, an epoxide derivative (XVI) of interest is obtained by conducting the reaction at −20 to 100° C., preferably at 0 to 30° C. for 30 minutes to 1 day, preferably for 3 hours to 15 hours.

A ring-opening reaction of the epoxide derivative (XVI) can be conducted in a solvent in the presence of a Lewis acid or in the presence of an oxidant. A solvent is not particularly limited, and for example, 1,2-dichloroethane, chloroform, methylene chloride, toluene, benzene or the like can be used. A Lewis acid is not particularly limited, and for example, boron trifluoride diethyl ether, titanium(IV) chloride, magnesium bromide, aluminum chloride, zinc bromide, berylium chloride or the like can be used. An oxidant is not particularly limited, and sodium periodate, orthoperiodic acid or the like can be used. The reaction condition varies depending on the materials used, but generally, an aldehyde derivative (XVII) of interest is obtained by conducting the reaction at −20 to 100° C., preferably at 0 to 30° C. for 5 minutes to 1 day, preferably for 2 to 12 hours.

A reaction of an aldehyde derivative (XVII) and a hydrazine derivative (XVIII) can be conducted in a solvent in the presence or absence of an acid. A solvent is not particularly limited, and for example, the followings can be used independently or in combination: tetrahydrofuran, toluene, dioxane, N,N-dimethylformamide, N-methylpyrrolidone, methylene chloride., acetonitrile, propionitrile and the like. An acid is not particularly limited, and for example, the followings can be used: hydrochloric acid, sulfuric acid, phosphoric acid, trifluoroacetic acid, difluoroacetic acid, fluoroacetic acid and the like. The reaction condition varies depending on the materials used, but generally, a substance of interest (XIX) is obtained by conducting the reaction at −20 to 150° C., preferably at 15 to 80° C. for 5 minutes to 2 days, preferably for 5 hours to 24 hours.

An oxidation reaction of the compound (XIX) can be conducted in a solvent by allowing an oxidant to act on the compound. A solvent is not particularly limited, and for example, 1,2-dichloroethane, chloroform, methylene chloride, toluene, benzene tetrahydrofuran, dioxane or the like can be used. An oxidant is not particularly limited, and for example, 3-chloroperbenzoic acid, peracetic acid, hydrogen peroxide, sodium periodate, orthoperiodic acid or the like can be used. The reaction condition varies depending on the materials used, but generally, a spiro oxindole compound (I) of interest is obtained by conducting the reaction at −20 to 100° C., preferably at 0 to 30° C. for 5 minutes to 1 day, preferably for 2 hours to 12 hours.

Further, a compound shown by formula (1) can also be produced according to the reaction formula below.

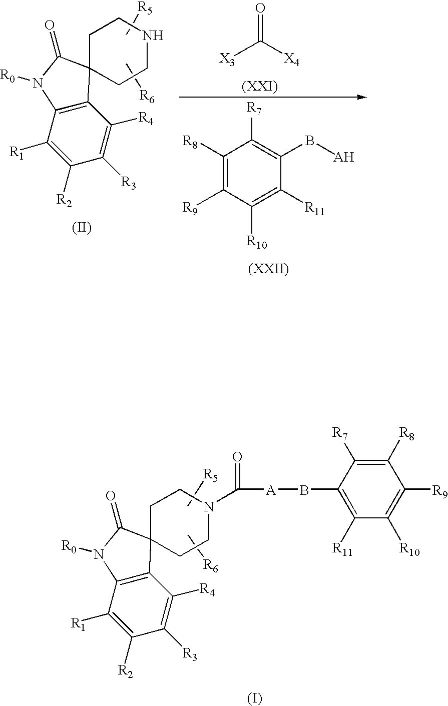

(wherein, R₀, R₁, R₂, R₃, R₄, R₅, R₆, R₇, R₈, R₉, R₁₀, R₁₁, A, and B show the same things as they show in the above general formula (I), and each X₃ and X₄ show a chlorine atom, imidazole, or aryloxy group).

A reaction of an amine compound (II) with a compound (XXII) can be conducted in a solvent in the presence or absence of a base, by allowing the three components (II), (XXI), and (XXII) to react at the same time. A solvent is not particularly limited, and for example, the followings can be used independently or in combination: tetrahydrofuran, toluene, dioxane, methylene chloride, acetonitrile and the like. A base is not particularly limited., and for example, the followings can be used: pyridine, collidine, lutidine, triethylamine, diisopropylethylamine, diisopropylpentylamine, trimethylamine and the like. The reaction condition varies depending on the materials used, but generally, a substance of interest (I) is obtained by conducting the reaction at −20 to 150° C., preferably at 0 to 80° C. for 5 minutes to 2 days, preferably for 1 to 12 hours.

### II. Method for Producing Compounds Represented by (II) or Salt Thereof, or Their Solvates

The spiro oxindoles (II) used in the above-mentioned production method can be produced according to methods known from literatures, or pursuant to those methods, for example, according to the chemical formula below.

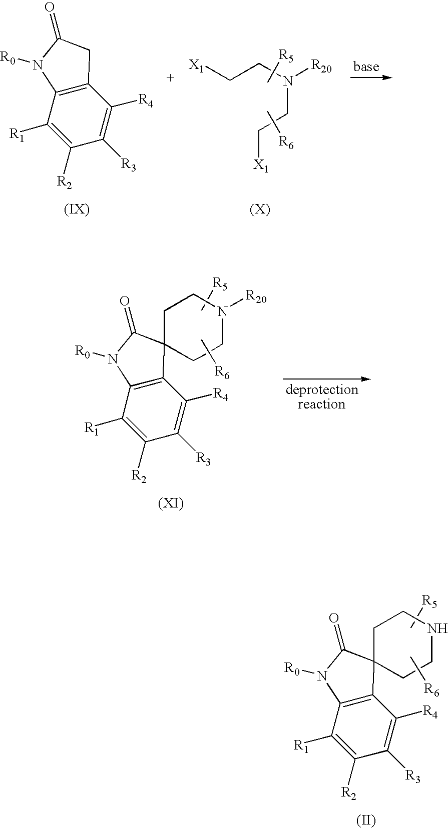

(wherein R₀, R₁, R₂, R₃, R₄, R₅, and R₆ show the same things as they show in the above-mentioned general formula (1), R₂₀ shows a protecting group, X₁ shows a halogen atom, alkylsulfonyloxy group, haloalkylsulfonyloxy group, or arylsulfonyloxy group).

A reaction of an oxindole derivative (IX) with amines (X) can be conducted in a solvent in the presence of a base. A solvent is not particularly limited, and for example, the followings can be used independently or in combination: tetrahydrofuran, toluene, dioxane, N,N-dimethylformamide, dimethylsulfoxide (DMSO), acetonitrile, propionitrile and the like. A base is not particularly limited, and for example, the followings can be used: alkali metal hydrides such as lithium hydride, sodium hydride, and potassium hydride; alkali metal carbonates such as lithium carbonate, sodium carbonate, potassium carbonate, and cesium carbonate; DABCO, lithium diisopropylamide, sodium diisopropylamide, potassium diisopropylamide, lithium hexamethyldisilazide, sodium hexamethyldisilazide, potassium hexamethyldisilazide, sodium t-butoxide, potassium t-butoxide, n-butyllithium, s-butyllithium, t-butyllithium and the like. The reaction condition varies depending on the materials used, but generally, a spiro oxindole (XI) is obtained by conducting the reaction at 0 to 150° C., preferably at 15 to 90° C. for 5 minutes to 1 day, preferably for 5 to 15 hours.

A deprotection of protecting group R₂₀ of the compound (XI) obtained in the above method is not particularly limited, which can be conducted with reference to a commonly used method (Protective Groups in Organic Synthesis Third Edition, John Wiley & Sons, Inc.) as a deprotection condition of the protecting group. A protecting group is not particularly limited, and for example, the followings can be used: a benzyl group, 9-fluorenylmethoxycarbonyl group (Fmoc group), 2,2,2-trichloroethoxycarbonyl group (Troc group), 2-(trimethylsilyl)ethoxycarbonyl group (Teoc group), t-butoxycarbonyl group (Boc group), allyloxycarbonyl group (Alloc group), vinyloxycarbonyl group, benzyloxycarbonyl group (Cbz group), p-methoxybenzyloxycarbonyl group, p-nitrobenzyloxycarbonyl group, allyl group, 2-(trimethylsilyl)ethoxymethyl group (SEM group), 4-methoxybenzyl group, triphenylmethyl group, benzenesulfonyl group, and o-nitrobenzenesulfonyl group. In particular, a benzyl group, Fmoc group, Boc group, and Cbz group are preferred.

The intermediates and substances of interest obtained in each of the above reactions can be isolated and purified as desired by subjecting to a purification method that are used routinely in the field of organic synthetic chemistry, for example, filtration, extraction, washing, drying, condensation, recrystallization, various types of chromatography and the like. Alternatively, the intermediates can be used for next reactions without a particular purification.

Further, various isomers can be isolated by applying a routine procedure utilizing the difference in physical-chemical property between the isomers. For example, a racemic mixture can be led to optically-pure isomers by a common racemic resolution method such as an optical resolution method comprising leading a mixture to diastereomeric salt with a common optically-active acid such as tartaric acid, or a method using optically-active column chromatography. Further, a diastereomeric mixture can be separated by a fractional crystallization, various types of chromatography or the like. Alternatively, an optically-active compound can be produced by using an appropriate optically-active material.

### EMBODIMENT OF USE

The pharmaceutical composition of the present invention comprises a spiro oxindole compound shown by general formula (I), pharmaceutically acceptable salt thereof, or their solvate as an active ingredient. The compound of the present invention can be used independently, but generally, the compound is used in combination with a pharmaceutically acceptable carrier and/or diluent.

Examples of an administration form of a medicine that comprises the compound of the present invention or pharmaceutically acceptable salt thereof, or their solvate as an active ingredient include an oral administration by a tablet, capsule., granules, powder, syrup or the like; or a parenteral administration by an intravenous injection, intramuscular injection, suppository, inhaler, percutaneous absorption, eye-drops, nasal preparation or the like. Further, to prepare a pharmaceutical formulation in such various forms, the active ingredient can be prepared independently or as a pharmaceutical composition where appropriate, by combining with other pharmaceutically acceptable carriers, specifically, an excipient, binder, extender, disintegrant, surfactant, lubricant, dispersant, buffer, preservative, flavoring agent, flavor, coating agent, diluent or the like.

The dose of the medicine of the present invention varies depending on weight, age, sex, symptom and the like of the patient, but generally, in a case of an adult, the compound represented by general formula (1) can be administered in an amount of 0.1 to 500 mg, in particular 1 to 300 mg a day, as a single or several separate doses either orally or parenterally.

Next, the present invention will be described further with reference to the following examples, while the scope of the present invention will not be limited to these examples.

### EXAMPLE 1

### Production of 1′-{2-(2-trifluoromethylphenoxy)acetyl}spiro(indole-3,4′-pi peridine)-2(1H)-one

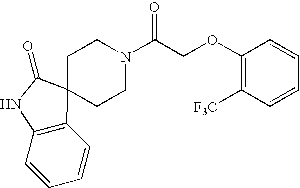

### Process 1

### Production of spiro(indole-3,4′-piperidine)-2(1H)-one

Spiro(indole-3,4′-piperidine)-2(1H)-one was produced by the method described below.

Under an argon atmosphere, a tetrahydrofuran solution (20 mL) of oxindole (3.00 g, 22.5 mmol) and a tetrahydrofuran solution (20 mL) of benzylbis(2-chloroethyl)amine (5.20 g, 22.5 mmol) were added sequentially to a tetrahydrofuran solution (100 mL) of sodium hydride (1.60 g, 67.6 mmol) at room temperature. The mixture was stirred at the same temperature for 1 hour and further stirred at 90° C. for 3 hours. The reaction solution was cooled to room temperature, and then added with a tetrahydrofuran solution (10 mL) of sodium hydride (0.540 g, 22.5 mmol) and stirred further at 90° C. for 12 hours. The reaction solution was added with saturated ammonium-chloride aqueous solution and stirred at room temperature for 10 minutes. The mixed solution was poured into the mixed solution of a saturated aqueous solution of sodium hydrogen carbonate and brine, then extracted with ethyl acetate. The organic layer was dried with anhydrous sodium sulfate, followed by a vacuum concentration. The resultant residue was purified by silica-gel chromatography (chloroform:methanol=20:1) and 1′-benzylspiro(indole-3,4′-piperidine)-2(1H)-one (2.94 g, 44.6%) was obtained as a yellow amorphous solid.

¹H-NMR (400 MHz, CDCl₃) δ; 1.78-1.93 (m, 2H), 1.95-2.06 (m, 2H), 2.65-2.77 (m, 2H), 2.87-3.00 (m, 2H), 3.69 (s, 2H), 6.90 (d, J=7.6 Hz, 1H), 7.02(t, J=7.6 Hz, 1H), 7.20 (t, J=7.6 Hz, 1H), 7.26 (t, J=6.2 Hz, 1H), 7.34 (t, J=7.6 Hz, 2H), 7.40-7.41(m, 3H), 8.72 (s, 1H).

To a methanol solution (5 mL) of 1′-benzylspiro(indole-3,4′-piperidine)-2(1H)-one (300 mg, 1.03 mmol), 10% palladium carbon (30.0 mg) was added. The mixture was stirred under a hydrogen atmosphere at room temperature for 15 hours. The reaction solution was filtered using celite followed by a vacuum concentration, and spiro(indole-3,4′-piperidine)-2(1H)-one (187 mg, 90.2%) was obtained as a colorless amorphous solid.

¹H-NMR (400 MHz, CDCl₃) δ; 1.73-1.78 (m, 2H), 1.88-1.94 (m, 2H), 3.06-3.12 (m, 2H), 3.35-3.41 (m, 2H), 6.92 (d, J=7.7 Hz, 1H), 7.04 (t, J=7.7 Hz, 1H), 7.22 (t, J=7.7 Hz, 1H), 7.42 (d, J=7.7 Hz, 1H), 8.70 (br, 1H).

### Process 2

### Production of 2-trifluoromethylphenoxyacetic acid

2-trifluoromethylphenoxyacetic acid was produced by the method described below.

To an N,N-dimethylformamide solution (5 mL) of 2-trifluoromethylphenol (300 mg, 1.85 mmol), ethyl bromoacetate (340 mg, 2.04 mmol) and potassium carbonate (384 mg, 2.78 mmol) was added at room temperature. The mixture was stirred at the same temperature for 15 hours. The reaction solution was added with water and extracted with ethyl acetate. The organic layer was washed with brine and dried with anhydrous sodium sulfate, followed by a vacuum concentration. The resultant residue was purified by silica-gel chromatography (hexane:ethyl acetate=2:1) and 2-trifluoromethylphenoxyethyl acetate (430 mg, 100%) was obtained as colorless oil.

¹H-NMR (400 MHz, CDCl₃) δ; 1.28(t, J=7.1 Hz, 3H), 4.26 (q, J=7.1 Hz, 2H), 4.72 (s, 2H), 6.88 (d, J=7.8 Hz, 1H), 7.06 (t, J=7.8 Hz, 1H), 7.47 (t, J=7.8 Hz, 1H), 7.60 (d, J=7.8 Hz, 1H).

To an ethanol solution (4 mL) of 2-trifluoromethylphenoxyethyl acetate (429 mg, 1.85 mmol), an aqueous solution of 4N-sodium hydroxide (1 mL) was added at room temperature. The mixture was stirred at the same temperature for 1.5 hours. The reaction solution was subjected to a vacuum concentration. The residue was added with 2N-hydrochloric acid and extracted with chloroform. The organic layer was dried with anhydrous sodium sulfate, followed by a vacuum concentration, and 2-trifluoromethylphenbxyacetic acid (376 mg, 92.3%) was obtained as white crystalline powder.

¹H-NMR (400 MHz, CD₃OD) δ; 4.79(s, 2H), 7.04-7.10 (m, 2H), 7.54 (t, J=7.8 Hz, 1H), 7.59 (d, J=7.8 Hz, 1H).

### Process 3

### Production of 1′-{2-(2-trifluoromethylphenoxy)acetyl}spiro(indole-3,4′-pi peridine)-2(1H)-one

1′-{2-(2-trifluoromethylphenoxy)acetyl}spiro(indole-3, 4′-piperidine)-2(1H)-one was produced by the method described below.

To a methylene chloride solution (3 mL) of 2-trifluoromethylphenoxyacetic acid (30.0 mg, 0.136 mmol), oxalyl chloride (34.6 mg, 0.273 mmol) and N,N-dimethylformamide (0.01 mL) were added at room temperature. The mixture was stirred at the same temperature for 1 hour. The reaction solution was subjected to a vacuum concentration and then the residue was added with methylene chloride (3 mL) and dissolved. To this solution, spiro-(indole-3,4′-piperidine)-2(1H)-one (27.6 mg, 0.136 mmol) and triethylamine (20.7 mg, 0.200 mmol) was added and the mixture was stirred at room temperature for hours. The reaction solution was added with water and then extracted with chloroform. The organic layer was dried with anhydrous sodium sulfate, followed by a vacuum concentration. The resultant residue was purified by preparative thin-layer chromatography (chloroform:methanol=10:1) and 1′-{2-(2-trifluoromethylphenoxy)acetyl}spiro(indole-3,4′-pi peridine)-2(1H)-one (22.0 mg, 39.9%) was obtained as colorless oil.

¹H-NMR (400 MHz, CDCl₃) δ; 1.78-1.89 (m, 4H), 3.79-3.86 (m, 1H), 3.97-4.12 (m, 2H), 4.23-4.27 (m, 1H), 4.83 (d, J=13.3 Hz, 1H), 4.93 (d, J=13.3 Hz, 1H), 6.91 (d, J=7.8 Hz, 1H), 7.01-7.11 (m, 3H), 7.17-7.24 (m, 2H), 7.54 (t, J=7.8 Hz, 1H), 7.61 (d, J=7.8 Hz, 1H), 8.49 (s, 1H).

IR(ATR); 3312,1706,1636,1470,1246,741 cm⁻¹.

EI-MS m/z; 404(M⁺).

### EXAMPLE 2

### Production of 1′-{2-(2-methylphenoxy)acetyl}spiro(indole-3,4′-piperidine)-2(1H)-one

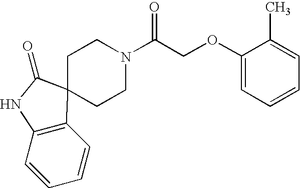

### Process 1

### Production of 1′-(2-bromoacetyl)spiro(indole-3,4′-piperidine)-2(1H)-one

1′-(2-bromoacetyl)spiro(indole-3,4′-piperidine)-2(1H)-one was produced by the method described below.

Bromoacetylbromide (175 mg, 0.870 mmol) and triethylamine mg, 0.950 mmol) were added to a mixed solution of N,N-dimethylformamide (1 mL) of spiro(indole-3,4′-piperidine)-2(1H)-one (175 mg, 0.870 mmol) and methylene chloride (5 mL) at room temperature and the resultant mixture was stirred at the same temperature for 2 hours. The reaction solution was added with water and extracted with chloroform. The organic layer was washed with brine and dried with anhydrous sodium sulfate, followed by a vacuum concentration. The resultant residue was purified by preparative thin-layer chromatography (chloroform:ethyl acetate=1:2), and 1′-(2-bromoacetyl)spiro(indole-3,4′-piperidine)-2(1H)-one (203 mg, 72.7%) was obtained as a pale yellow amorphous solid.

¹H-NMR (400 MHz, CDCl₃) δ; 1.83-2.00 (m, 4H), 3.76-3.79 (m, 1H), 3.85-4.01 (m, 2H), 4.06-4.25 (m, 3H), 6.97 (d, J=7.6 Hz, 1H), 7.05 (t, J=7.6 Hz, 1H), 7.22-7.25 (m, 2H), 9.32 (s, 1H).

### Process 2

### Production of 1′-{2-(2-methylphenoxy)acetyl}spiro(indole-3,4′-piperidine)-2(1H)-one

1′-{2-(2-methylphenoxy)acetyl}spiro(indole-3,4′-piper idine)-2(1H)-one was produced by the method described below.

To an N,N-dimethylformamide solution (2.00 mL) of 1′-(2-bromoacetyl)spiro(indole-3,4′-piperidine)-2(1H)-one mg, 0.124 mmol), ortho-cresol (13.4 mg, 0.124 mmol) and potassium carbonate (25.7 mg, 0.186 mmol) were added sequentially at room temperature. The mixture was stirred at the same temperature for 12 hours. The reaction solution was added with water and extracted with ethyl acetate. The organic layer was dried with anhydrous sodium sulfate, followed by a vacuum concentration. The resultant residue was purified by preparative thin-layer chromatography (chloroform:ethyl acetate=1:2), and 1′-{2-(2-methylphenoxy)acetyl}spiro(indole-3,4′-piperidine)-2(1H)-one (5.70 mg, 13.1%) was obtained as a white amorphous solid.

¹H-NMR (400 MHz, CDCl₃) δ; 1.78-1.90 (m, 4H), 2.26(s, 3H), 3.75-3.85 (m, 1H), 3.95-4.10 (m, 2H), 4.26-4.35 (m, 1H), 4.74 (d, J=13.2 Hz, 1H), 4.82(d, J=13.2 Hz, 1H), 6.86-6.95 (m, 3H), 7.00-7.05 (m, 2H), 7.16-7.24 (m, 3H), 7.49 (s, 1H).

IR(ATR); 3205,1706,1644,1471,1253,746 cm⁻¹.

EI-MS m/z; 350(M⁺).

### EXAMPLE 3

### Production of 1′-{2-(2-trifluoromethoxyphenoxy)acetyl}spiro(indole-3,4′-piperidine)-2(1H)-one

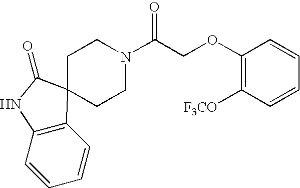

1′-{2-(2-trifluoromethoxyphenoxy)acetyl}spiro(indole-3,4′-piperidine)-2(1H)-one was produced by the method described below.

### Process 1

### Production of 2-trifluoromethoxyphenoxyacetic acid

The reaction and treatment were conducted in a similar manner to process 2 of example 1 using 2-trifluoromethoxyphenol in place of 2-trifluoromethylphenol, and 2-trifluoromethoxyphenoxyacetic acid was obtained as white crystalline powder.

¹H-NMR (400 MHz, CDCl₃) δ; 3.10(brs, 1H), 4.74 (s, 2H), 6.96 (d, J=8.0 Hz, 1H), 7.05 (t, J=7.7 Hz, 1H), 7.24-7.30 (m, 2H).

### Process 2

### Production of 1′-{2-(2-trifluoromethoxyphenoxy)acetyl}spiro(indole-3,4′-piperidine)-2(1H)-one

To a methylene chloride solution (5 mL) of spiro (indole-3,4′-piperidine)-2(1H)-one (50.0 mg, 0.247 mmol), 2-trifluoromethoxyphenoxyacetic acid (58.4 mg, 0.247 mmol), PyBOP (142 mg, 0.272 mmol), and diisopropylethylamine (63.9 mg, 0.494 mmol) were added sequentially at room temperature. The mixture was stirred at the same temperature for 5 hours. The reaction solution was added with water and then extracted with chloroform. The organic layer was dried with anhydrous sodium sulfate, followed by a vacuum concentration. The resultant residue was purified by preparative thin-layer chromatography (hexane:ethyl acetate=1:2) and 1′-{2-(2-trifluoromethoxyphenoxy)acetyl}spiro(indole-3,4′-piperidine)-2(1H)-one (33.0 mg, 31.8%) was obtained as a colorless amorphous solid.

¹H-NMR (400 MHz, CDCl₃) δ; 1.83-1.91 (m, 4H), 3.80-3.86 (m, 1H), 3.90-3.97 (m, 1H), 4.03 (quint, J=6.8 Hz, 1H), 4.24-4.30 (m, 1H), 4.79 (d, J=12.9 Hz, 1H), 4.87 (d, J=12.9 Hz, 1H), 6.91 (t, J=7.8 Hz, 1H), 7.00-7.30 (m, 7H), 8.58 (s, 1H).

IR(ATR); 3217,1711,1632,1471,1223,755 cm⁻¹.

EI-MS m/z; 420(M⁺).

### EXAMPLE 4

### Production of 1′-{2-(2-trifluoromethylphenoxy)acetyl}spiro(5-chloroindole-3,4′-piperidine)-2(1H)-one

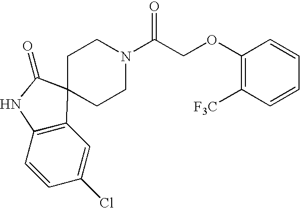

The reaction and treatment were conducted in a similar manner to example 3 using 5-chlorospiro(indole-3,4′-piperidine)-2(1H)-one produced by using 5-chloro-oxyindole in place of oxyindole in process 1 of example 1, in place of spiro (indole-3,4′-piperidine)-2(1H)-one; and also using 2-trifluoromethylphenoxyacetic acid in place of 2-trifluoromethoxyphenoxyacetic acid, respectively, and the title compound was obtained as a colorless amorphous solid.

¹H-NMR (400 MHz, CDCl₃) δ; 1.73-1.88 (m, 4H), 3.75-3.82 (m, 1H), 3.98-4.10 (m, 2H), 4.24-4.29 (m, 1H), 4.84 (d, J=13.4 Hz, 1H), 4.93 (d, J=13.4 Hz, 1H), 6.84 (d, J=8.3 Hz, 1H), 6.97 (d, J=2.0 Hz, 1H), 7.11 (t, J=7.6 Hz, 1H), 7.19 (d, J=8.3 Hz, 2H), 7.55 (t, J=7.2 Hz, 1H), 7.62 (d, J=7.8 Hz, 1H), 8.60 (s, 1H).

IR(ATR); 3222,1709,1645,1321,1117,755 cm⁻¹.

EI-MS m/z; 43.8[M+].

### EXAMPLE 5

### Production of 1′-{2-(4-methoxyphenoxy)-2-methylpropionyl}spiro(indole-3,4′-piperidine)-2(1H)-one

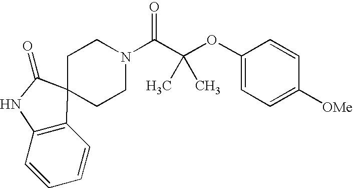

The reaction and treatment were conducted in a similar manner to example 3 using 2-(4-methoxyphenoxy)-2-methylpropionic acid in place of 2-trifluoromethoxyphenoxyacetic acid, and the title compound was obtained as a colorless amorphous solid.

¹H-NMR (400 MHz, CDCl₃) δ; 1.28-1.37 (m, 1H) 1.56-1.79 (m, 3H), 1.66 (s, 3H), 1.68 (s, 3H), 3.75-3.78 (m, 1H), 3.81 (s, 3H), 4.03-4.12 (m 8(t, J=7.7 Hz, 1H), 8.21 (s, 1H).

IR(ATR); 3179,1693,1628,1504,1221,1036 cm⁻¹.

EI-MS m/z; 394[M+].

### EXAMPLE 6

### Production of N-[4-{5-chloro-2-oxospiro(indole-3,4′-piperidine)-1′-yl)-4-oxobutyl]-2-(4-chlorophenyl)acetamide

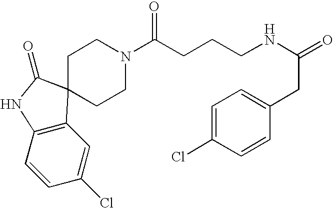

### Process 1

### Production of 4-(2-(4-chlorophenyl)acetamide}butanoic acid

4-{2-(4-chlorophenyl)acetamide}butanoic acid was synthesized by the method described below.

To a methylene chloride solution (5 mL) of 4-ethyl aminobutanoate hydrochloride (500 mg, 2.98 mmol), 4-chlorophenylacetylchloride (564 mg, 2.98 mmol) and triethylamine (905 mg, 8.95 mmol) was added sequentially at room temperature. The mixture was stirred at the same temperature for 12 hours. The reaction solution was added with water and extracted with chloroform. The organic layer was dried with anhydrous sodium sulfate, followed by a vacuum concentration. The resultant residue was purified by silica-gel chromatography (hexane:ethyl acetate=1:1) and 4-{2-(4-chlorophenyl)acetamide}ethyl butanoate (541 mg, 63.9%) was obtained as colorless needle-like crystals.

¹H-NMR (400 MHz, CDCl₃) δ; 1.24(t, J=7.2 Hz, 3H), 1.78 (quint J=7.10 (q, J=7.2 Hz, 2H), 5.65 (brs, 1H), 7.20 (d, J=8.5 Hz, 2H), 7.33 (d, J=8.5 Hz, 2H).

To an ethanol solution (3 mL) of 4-{2-(4-chlorophenyl)acetamide}ethyl butanoate (540 mg, 1.90 mmol), an aqueous solution of 4N-sodium hydroxide (2 mL) was added at room temperature. The mixture was stirred at the same temperature for 15 minutes. The reaction solution was subjected to a vacuum concentration, added with water and then washed with diethylether. The aqueous layer was added with 1N-hydrochloric acid, adjusted to pH=1.0, and then extracted with chloroform. The organic layer was dried with anhydrous sodium sulfate, followed by a vacuum concentration, and 4-{2-(4-chlorophenyl)acetamide}butanoic acid (421 mg, 86.4%) was obtained as a colorless amorphous solid.

### Process 2

### Production of N-[4-{5-chloro-2-oxospiro(indole-3,4′-piperidine)-1′-yl}-4-oxobutyl]-2-(4-chlorophenyl)acetamide

The reaction and treatment were conducted in a similar manner to example 3 using 5-chlorospiro(indole-3,4′-piperidine)-2(1H)-one in place of spiro(indole-3,4′-piperidine)-2(1H)-one, and 4-{2-(4-chlorophenyl)acetamide}butanoic acid in place of 2-trifluoromethoxyphenoxyacetic acid, and the title compound was obtained as a clolorless amorphous solid.

¹H-NMR (400 MHz, CDCl₃) δ; 1.73-1.89 (m, 6H), 2.42 (t, J=6.7 Hz, 2H), 3.29-3.33 (m, 2H), 3.52 (s, 2H), 3.63-3.82 (m, 2H), 3.91-3.97 (m, 1H), 4.11-4.17 (m, 1H), 6.42-6.46 (m, 1H), 6.85 (d, J=7.8 Hz, 1H), 7.20-7.24 (m, 4H), 7.29-7.31 (m, 3H), 8.63 (s, 1H).

IR(ATR); 3296,1708,1621,1479,1091,755 cm⁻¹.

EI-MS m/z; 473[M+].

### EXAMPLE 7

### Production of 1′-{(3-phenoxy) propionyl}spiro(indole-3,4′-piperidine)-2(1H)-one

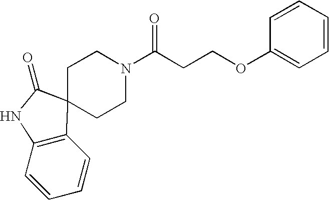

The reaction and treatment were conducted in a similar manner to example 3 using 3-phenoxypropionic acid in place of 2-trifluoromethoxyphenoxyacetic acid, and the title compound was obtained as a white crystalline solid.

¹H-NMR (400 MHz, CDCl₂) δ; 1.80-1.97 (m, 4H), 2.85-3.01 (m, 2H), 3.81-3.88 (m, 2H), 4.05-4.16 (m, 1H), 4.24-4.29 (m, 1H), 4.34-4.46 (m, 2H), 6.92-6.98 (m, 4H), 7.04 (t, J=7.6 Hz, 1H), 7.19-7.31 (m, 4H), 8.57 (s, 1H).

IR(ATR); 3190,2918,1690,1640,1468,1243,1181,1044,752 cm⁻¹.

EI-MS m/z; 350(M⁺).

### EXAMPLE 8

### Production of

### 1′-{5-(2-trifluoromethylphenoxy)pentanoyl}spiro(indole-3,4′-piperidine)-2(1H)-one

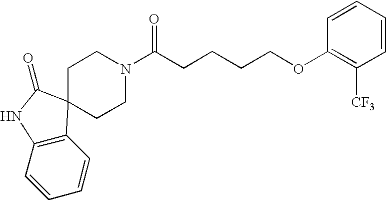

The reaction and treatment were conducted in a similar manner to example 3 using 5-trifluorophenoxypentanoic acid in place of 2-trifluoromethoxyphenoxyacetic acid, and the title compound was obtained as a white amorphous solid.

¹H-NMR (400 MHz, CDCl₃) δ; 1.79-1.94 (m, 8H), 2.52 (t, J=6.1 Hz, 2H), 3.71-3.79 (m, 1H), 3.81-3.88 (m, 1H), 3.97-4.04 (m, 1H), 4.10-4.15 (m, 2H), 4.17-4.24 (m, 1H), 6.91 (d, J=7.8 Hz, 1H), 6.97-7.01 (m, 2H), 7.05 (t, J=7.6 Hz, 1H), 7.22-7.26 (m, 2H), 7.48 (t, J=7.6 Hz, 1H), 7.55 (d, J=7.6 Hz, 1H), 7.93 (s, 1H).

IR(ATR); 3201,2941,1706,1608,1472,1459,1230,1116,1037,753 cm⁻¹.

EI-MS m/z; 446(M⁺).

### EXAMPLE 9

### Production of 1′-{2-(2-methoxyphenoxy)acetyl}spiro(indole-3,4′-piperidine)-2(1H) one

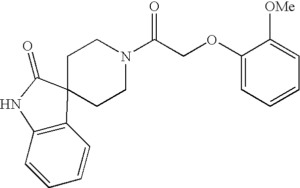

The reaction and treatment were conducted in a similar manner to process 2 of example 2 using 2-methoxyphenol in place of ortho-cresol, and the title compound was obtained as a white amorphous solid.

¹H-NMR (400 MHz, CDCl₃) δ; 1.76-1.89 (m, 4H), 3.75-3.82 (m, 1H) 3.84 (s, 3H), 3.99-4.11 (m, 2H), 4.25-4.28 (m, 1H), 4.79 (d, J=13.3 Hz, 1 H), 4.86(d, J=13.3 Hz, 1H), 6.88-6.95 (m, 3H), 6.99-7.06 (m, 4H), 7.20-7.26 (m, 1H), 7.98 (s, 1H).

IR(ATR); 3228,1706,1644,1471,1253,746 cm⁻¹.

EI-MS m/z; 366(M⁺).

### EXAMPLE 10

### Production of 1′-{2-(2-iodophenoxy)acetyl}spiro(indole-3,4′-piperdine)-2 (1H)-one

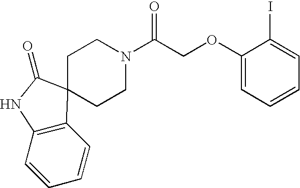

The reaction and treatment were conducted in a similar manner to process 2 of example 2 using 2-iodophenol in place of ortho-cresol, and the title compound was obtained as a white amorphous solid.

¹H-NMR (400 MHz, CDCl₃) δ; 1.76-1.92 (m, 4H), 3.78-3.83 (m, 1H), 4.00-4.10 (m, 2H), 4.25-4.29 (m, 1H), 4.79(d, J=13.2 Hz, 1H), 4.90 (d, J=13.2 Hz, 1H), 6.79 (t, J=7.8 Hz, 1H), 6.90 (d, J=7.8 Hz, 1H), 6.99-7.03 (m, 3H), 7.20-7.24 (m, 1H), 7.34 (t, J=7.8 Hz, 1H), 7.80 (d, J=7.8 Hz, 1H), 8.21(s, 1H).

IR(ATR); 3234,1704,1640,1471,1227,747 cm⁻¹.

EI-MS m/z; 462(M⁺).

### EXAMPLE 11

### Production of 1′-{2-(2-cyanophenoxy)acetyl}spiro(indole-3,4′-piperidine)-2(1H)-one

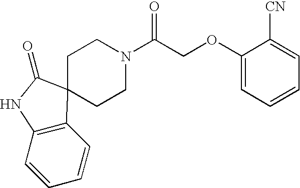

The reaction and treatment were conducted in a similar manner to process 2 of example 2 using 2-cyanophenol in place of ortho-cresol, and the title compound was obtained as a white amorphous solid.

¹H-NMR (400 MHz, CDCl₃) δ; 1.82-1.85 (m, 2H), 1.95-1.98 (m, 2H), 3.78-3.85 (m, 1H), 3.92-3.97 (m, 1H), 4.09-4.15(m, 1H), 4.24-4.29 (m, 1H), 4.87 (d, J=13.4 Hz, 1H), 4.97 (d, J=13.4 Hz, 1H), 6.90 (d, J=7.8 Hz, 1H), 7.02-7.15 (m, 4H), 7.22 (t, J=7.8 Hz, 1H), 7.55-7.61 (m, 2H), 8.20 (s, 1H).

IR(ATR); 3234,1705,1647,1472,1229,751 cm⁻¹.

EI-MS m/z; 361(M⁺).

### EXAMPLE 12

### Production of 1′-{2-(2-nitrophenoxy)acetyl}spiro(indole-3,4′-piperidine)-2(1H)-one

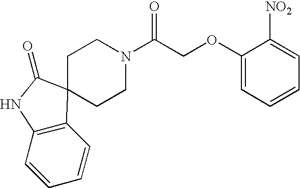

The reaction and treatment were conducted in a similar manner to process 2 of example 2 using 2-nitrophenol in place of ortho-cresol, and the title compound was obtained as a white amorphous solid.

¹H-NMR (400 MHz, CDCl₃) δ; 1.82-1.92 (m, 4H), 3.79-3.85 (m, 1H), 3.92-3.99 (m, 1H), 4.08-4.15 (m, 1H), 4.22-4.28 (m, 1H), 4.87 (d, J=13.4 Hz, 1H), 4.8 (d, J=13.4 Hz, 1H), 6.89 (d, J=7.8 Hz, 1H), 7.44 (t, J=7.8 Hz, 1H), 7.10-7.14 (m, 2H), 7.21-7.27 (m, 2H), 7.57 (t, J=7.8 Hz, 1H), 7.88 (d, J=7.8 Hz, 1H), 7.94 (s, 1H).

IR(ATR); 3229,1706,1647,1523,1229,744 cm⁻¹.

EI-MS m/z; 381(M⁺).

### EXAMPLE 13

### Production of 1′-{2-(4-nitrophenoxy)acetyl}spiro(indole-3,4′-piperidine)-2(1H)-one

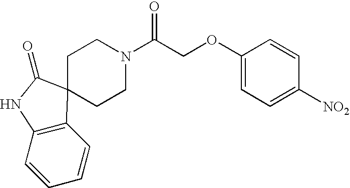

The reaction and treatment were conducted in a similar manner to process 2 of example 2 using 4-nitrophenol in place of ortho-cresol, and the title compound was obtained as a white amorphous solid.

¹H-NMR (400 MHz, CDCl₃) δ; 1.78-1.97 (m, 4H), 3.79-3.89 (m, 2H), 4.07-4.12 (m, 1H), 4.21-4.27 (m, 1H), 4.88 (d, J=13.8 Hz, 1H), 4.93 (d, J=13.8 Hz, 1H), 6.92 (d, J=7.6 Hz, 1H), 7.03-7.14 (m, 4H), 7.26 (t, J=7.6 Hz, 1H), 8.21-8.25 (m, 2H), 8.38(s, 1H).

IR(ATR); 3245,1696,1651,1341,1237,763 cm⁻¹.

EI-MS m/z; 381(M⁺).

### EXAMPLE 14

### Production of 1′-{2-(3-cyanophenoxy)acetyl}spiro(indole-3,4′-piperidine)-2(1H)-one

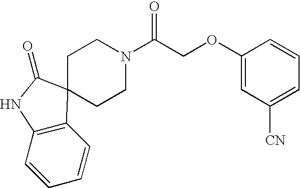

The reaction and treatment were conducted in a similar manner to process 2 of example 2 using 3-cyanophenol in place of ortho-cresol, and the title compound was obtained as a white amorphous solid.

¹H-NMR (400 MHz, CDCl₃) δ; 1.80-1.97 (m, 4H), 3.77-3.89 (m, 2H), 4.05-4.12 (m, 1H), 4.22-4.28 (m, 1H), 4.79 (d, J=13.8 Hz, 1H), 4.85 (d, J=13.8 Hz, 1H), 6.92 (d, J=7.8 Hz, 1H), 7.06(t, J=7.8 Hz, 1H), 7.15 (d, J=7.8 Hz, 1H), 7.23-7.27 (m, 3H), 7.31 (d, J=7.8 Hz, 1H), 7.42 (t, J=7.8 Hz, 1H), 8.33 (s, 1H).

IR(ATR); 3237,1706,1648,1472,1233,749 cm⁻¹.

EI-MS m/z; 361(M⁺).

### EXAMPLE 15

### Production of 1′-{2-(2,6-diphenylphenoxy)acetyl}spiro(indole-3,4′-piperid ine)-2(1H)-one

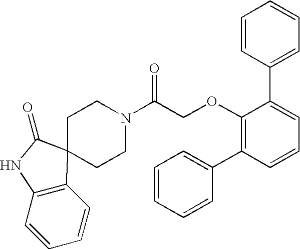

The reaction and treatment were conducted in a similar manner to process 2 of example 2 using 2,6-diphenylphenol in place of ortho-cresol, and the title compound was obtained as a white amorphous solid.

¹H-NMR (400 MHz, CDCl₃) δ; 1.44-1.84 (m, 4H), 3.18-3.24 (m, 2H), 3.51-3.62 (m, 2H), 3.80-3.86 (m, 1H), 3.93 (d, J=12.3 Hz, 1H), 3.98 (d, J=12.3 Hz, 1H), 6.88 (d, J=7.8 Hz, 1H), 7.04-7.05 (m, 2H), 7.24-7.47 (m, 10H), 7.60-7.62 (m, 4H), 8.44 (s, 1H).

IR(ATR); 3207,1709,1646,1471,1182,750 cm⁻¹.

EI-MS m/z; 488[M+].

### EXAMPLE 16

### Production of 1′-{2-(4-ethylphenoxy)acetyl}spiro(indole-3,4′-piperidine)-2(1H)-one

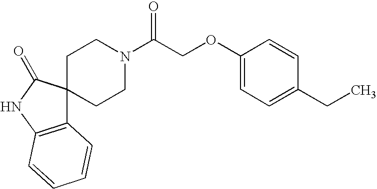

The reaction and treatment were conducted in a similar manner to process 2 of example 2 using 4-ethylphenol in place of ortho-cresol, and the title compound was obtained as a white amorphous solid.

¹H-NMR (400 MHz, CDCl₃) δ; 1.23(t, J=7.7 Hz, 3H) 1.79-1.87 (m, 4H), 2.52 (q, J=7.7 Hz, 2H), 3.75-3.83 (m, 1H), 3.90-3.95 (m, 1H), 4.01-4.07 (m, 1H), 4.25-4.31 (m, 1H), 4.71 (d, J=13.0 Hz, 1H), 4.78 (d, J=13.0 Hz, 1H), 6.86-6.93 (m, 3H), 7.02-7.03 (m, 2H), 7.13-7.15 (m, 2H), 7.20-7.24(m, 1H), 7.46 (s, 1H).

### EXAMPLE 17

### Production of 1′-{2-(4-fluorophenoxy)acetyl}spiro(indole-3,4′-piperidine)-2(1H)-one

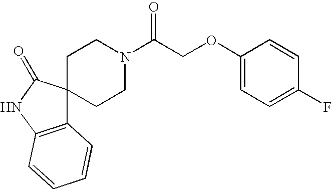

The reaction and treatment were conducted in a similar manner to process 2 of example 2 using 4-fluorophenol in place of ortho-cresol, and the title compound was obtained as a white amorphous solid.

¹H-NMR (400 MHz, CDCl₃) δ; 1.80-1.93 (m, 4H), 3.79-3.90(m, 2H) 4.03-4.09 (m, 1H), 4.23-4.28 (m, 1H), 4.71 (d, J=13.4 Hz., 1H), 4.77 (d, J=13.4 Hz, 1H), 6.89-7.09 (m, 6H), 7.19-7.25 (m, 2H), 7.85 (s, 1H).

IR(ATR); 3204,1696,1652,1504,1206,761 cm⁻¹.

EI-MS m/z; 354(M⁺).

### EXAMPLE 18

### Production of 1′-{2-(4-bromophenoxy)acetyl}spiro(indole-3,4′-piperidine)-2(1H)-one

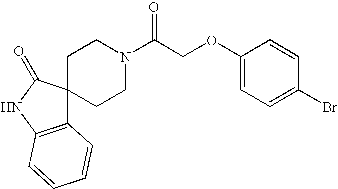

The reaction and treatment were conducted in a similar manner to process 2 of example 2 using 4-bromophenol in place of ortho-cresol, and the title compound was obtained as a white amorphous solid.

¹H-NMR (400 MHz, CDCl₂) δ; 1.76-1.92 (m, 4H), 3.78-3.89 (m, 2H), 4.02-4.09 (m, 1H), 4.22-4.28 (m, 1H), 4.73 (d, J=13.2 Hz, 1H), 4.79 (d, J=13.2 Hz, 1H), 6.88-6.92 (m, 3H), 7.03-7.08 (m, 2H), 7.21-7.25 (m, 1H), 7.40-7.42 (m, 2H), 8.20 (s, 1H).

IR(ATR); 3215,1707,1652,1487,1226,758 cm⁻¹.

EI-MS m/z; 414(M⁺).

### EXAMPLE 19

### Production of 1′-{2-(4-bromo-2,6-dimethylphenoxy)acetyl}spiro(indole-3,4′-piperidine)-2(1H)-one

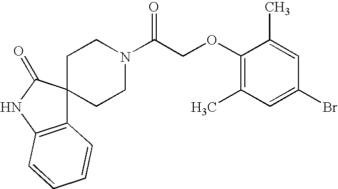

The reaction and treatment were conducted in a similar manner to process 2 of example 2 using 4-bromo-2,6-dimethylphenol in place of ortho-cresol, and the title compound was obtained as a white amorphous solid.

¹H-NMR (400 MHz, CDCl₂) δ; 1.86-1.97 (m, 4H), 2.30 (m, 6H), 3.79-3.84 (m, 1H), 3.88-3.93 (m, 1H), 4.05-4.12 (m 1H), 4.25-4.30 (m, 1H) 4.46 (d, J=12.7 Hz, 1H), 4.57 (d, J=12.7 Hz, 1H), 6.93 (d, J=7.6 Hz, 1H), 7.06 (t, J=7.6 Hz, 1H), 7.16 (s, 2H), 7.23 (d, J=7.6 Hz, 2H), 8.19 (s, 1H).

IR(ATR); 3235,1707,1646,1471,1182,748 cm⁻¹.

EI-MS m/z; 442(M⁺).

### EXAMPLE 20

### Production of 1′-{2-(4-idoophenoxy)acetyl}spiro(indole-3,4′-piperidine)-2 (1H)-one

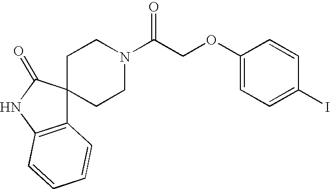

The reaction and treatment were conducted in a similar manner to process 2 of example 2 using 4-iodophenol in place of ortho-cresol, and the title compound was obtained as a white amorphous solid.

¹H-NMR (400 MHz, CDCl₃) δ; 1.75-1.89 (m, 4H), 3.77-3.88 (m, 2H), 4.01-4.08 (m, 1H), 4.23-4.28 (m, 1H), 4.71 (d, J=13.4 Hz, 1H), 4.78 (d, J=13.4 Hz, 1H), 6.76-6.80 (m, 2H), 6.90 (d, J=7.6 Hz, 1H), 7.05-7.06 (m, 2H), 7.21-7.25 (m, 1H), 7.58-7.61 (m, 2H), 8.13 (s, 1H).

IR(ATR); 3238,1705,1646,1485,1227,749 cm¹.

EI-MS m/z; 462(M⁺).

### EXAMPLE 21

### Production of 1′-{2-(4-methoxyphenoxy)acetyl}spiro(indole-3,4′-piperidine)-2(1H)-one

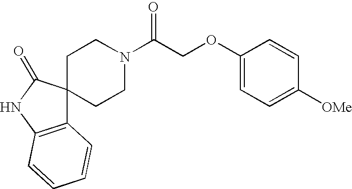

The reaction and treatment were conducted in a similar manner to process 2 of example 2 using 4-methoxyphenol in place of ortho-cresol, and the title compound was obtained as a white amorphous solid.

¹H-NMR (400 MHz, CDCl₂) δ; 1.81-1.91 (m, 4H), 3.76-3.81 (m, 2H), 3.79 (s, 3H), 3.83-3.93 (m, 1H), 4.01-4.10 (m, 1H), 4.24-4.30 (m, 1H), 4.69 (d, J=13.8 Hz, 1H), 4.75 (d, J=13.8 Hz, 1H), 6.85-6.95 (m, 4H), 7.02-7.08(m, 2H), 7.20-7.25 (m, 2H), 7.49 (s, 1H).

### EXAMPLE 22

### Production of 1′-{2-(3-methoxyphenoxy)acetyl}spiro(indole-3,4′-piperidine)-2(1H)-one

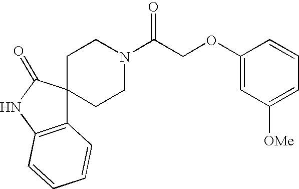

The reaction and treatment were conducted in a similar manner to process 2 of example 2 using 3-methoxyphenol in place of ortho-cresol, and the title compound was obtained as a white amorphous solid.

¹H-NMR (400 MHz, CDCl₃) δ; 1.78-1.89 (m, 4H), 3.77-3.90 (m, 2H), 3.80 (s, 3H), 4.02-4.09 (m, 1H), 4.25-4.30 (m, 1H), 4.74(d, J=13.1 Hz, 1H), 4.78 (d, J=13.1 Hz, 1H), 6.57-6.60 (m, 3H), 6.89(d, J=7.6 Hz, 1H), 7.02-7.09 (m, 2H), 7.20-7.25 (m, 2H), 7.76 (s, 1H).

IR(ATR); 3238,1706,1647,1471,1254,757 cm⁻¹.

EI-MS m/z; 366(M⁺).

### EXAMPLE 23

### Production of 1′-{2-(4-chlorophenoxy)acetyl}spiro(indole-3,4′-piperidine)-2(1H)-one

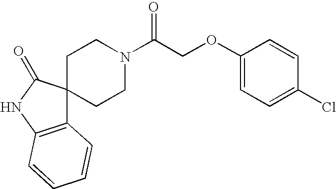

The reaction and treatment were conducted in a similar manner to process 2 of example 2 using 4-chlorophenol in place of ortho-cresol, and the title compound was obtained as a white amorphous solid.

¹H-NMR (400 MHz, CDCl₃) δ; 1.76-1.93 (m, 4H), 3.79-3.88 (m, 2H), 4.02-4.09 (m, 1H), 4.23-4.27 (m, 1H), 4.73 (d, J=13.4 Hz, 1H), 4.79 (d, J=13.4 Hz, 1H), 6.90-6.95 (m, 3H), 7.03-7.09 (m, 2H), 7.21-7.28 (m, 3H), 8.21(s, 1H).

IR(ATR); 3215,1710,1655,1488,1226,764 cm⁻¹.

EI-MS m/z; 370(M⁺).

### EXAMPLE 24

### Production of 1′-{2-(4-chloro-3-methylphenoxy)acetyl}spiro(indole-3,4′-pi peridine)-2(1H)-one

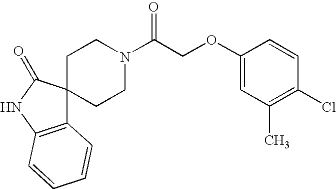

The reaction and treatment were conducted in a similar manner to process 2 of example 2 using 4-chloro-3-methylphenol in place of ortho-cresol, and the title compound was obtained as a white amorphous solid.

¹H-NMR (400 MHz, CDCl₃) δ; 1.76-1.92 (m, 4H), 2.35 (s, 3H), 3.78-3.88 (m, 2H), 4.02-4.08 (m, 1H), 4.24-4.28 (m, 1H), 4.71 (d, J=13.3 Hz, 1 H), 4.77 (d, J=13.3 Hz, 1H), 6.76-6.79 (m, 1H), 6.87-6.91 (m, 2H), 7.03-7.09(m, 2H), 7.21-7.27(m, 2H), 7.98 (s, 1H).

IR(ATR); 3238,1706,1647,1472,1231,748 cm⁻¹.

EI-MS m/z; 384(M⁺).

### EXAMPLE 25

### Production of 1′-{2-(3-chlorophenoxy)acetyl}spiro(indole-3,4′-piperidine)-2(1H)-one

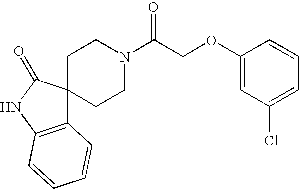

The reaction and treatment were conducted in a similar manner to process 2 of example 2 using 3-chlorophenol in place of ortho-cresol, and the title compound was obtained as a white amorphous solid.

¹H-NMR (400 MHz, CDCl₃) δ; 1.78-1.94 (m, 4H), 3.81-3.87 (m, 2H), 4.03-4.10 (m, 1H), 4.24-4.28 (m, 1H), 4.74 (d, J=13.3 Hz, 1H), 4.80 (d, J=13.3 Hz, 1H), 6.89-6.93 (m, 2H), 6.98-7.12 (m, 4H), 7.22-7.26 (m, 2H), 8.43(s, 1H).

IR(ATR); 3229,1706,1647,1472,1226,749 cm⁻¹

EI-MS m/z; 370(M⁺).

### EXAMPLE 26

### Production of 1′-{2-(3-nitrophenoxy)acetyl}spiro(indole-3,4′-piperidine)-2(1H)-one

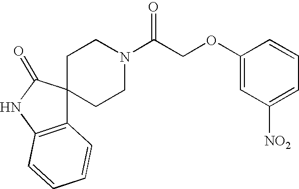

The reaction and treatment were conducted in a similar manner to process 2 of example 2 using 3-nitrophenol in place of ortho-cresol, and the title compound was obtained as a white amorphous solid.

¹H-NMR (400 MHz, CDCl₃) δ; 1.86-1.99 (m, 4H), 3.78-3.90 (m, 2H), 4.08-4.15 (m, 1H), 4.25-4.30 (m, 1H), 4.86 (d, J=13.5 Hz, 1H), 4.91(d, J=13.5 Hz, 1H), 6.93 (d, J=7.6 Hz, 1H), 7.06 (t, J=7.6 Hz, 1H), 7.17-7.27 (m, 2H), 7.35-7.38 (m, 1H), 7.48 (t, J=8.3 Hz, 1H) 7.79-7.80 (m, 1H), 7.88-7.90 (m, 1H), 8.43 (s, 1H).

IR(ATR); 3211,1706,1648,1527,1351,737 cm⁻¹.

EI-MS m/z; 381(M⁺).

### EXAMPLE 27

### Production of 1′-(2-phenoxyacetyl)spiro(indole-3′,4′-piperidine)-2(1H)-one

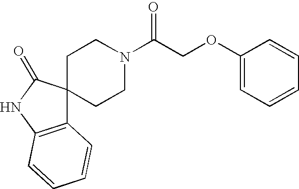

The reaction and treatment were conducted in a similar manner to process 2 of example 2 using phenol in place of ortho-cresol, and the title compound was obtained as a red-brown amorphous solid.

¹H-NMR (400 MHz, CDCl₃) δ; 1.79-1.89 (m, 4H), 3.76-3.83 (m, 1H), 3.88-3.94 (m, 1H), 4.02-4.09 (m, 1H), 4.25-4.31 (m, 1H), 4.74 (d, J=13. 1 Hz, 1H), 4.81 (d, J=13.1 Hz, 1H), 6.88 (d, J=7.6 Hz, 1H), 6.99-7.04 (m, 5H), 7.20-7.26 (m, 1H), 7.31-7.35 (m, 2H), 7.62 (s, 1H).

### EXAMPLE 28

### Production of 1′-(2-thiophenoxyacetyl)spiro(indole-3,4′-piperidine)-2(1H)-one

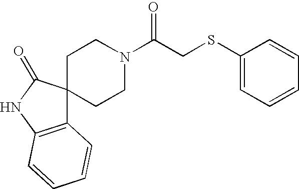

The reaction and treatment were conducted in a similar manner to process 2 of example 2 using thiophenol in place of ortho-cresol, and the title compound was obtained as a red-brown amorphous solid.

¹H-NMR (400 MHz, CDCl₃) δ; 1.81-1.92 (m, 4H), 3.73-3.89 (m, 4H), 4.01-4.06 (m, 1H), 4.20-4.24 (m, 1H), 6.92 (d, J=7.6 Hz, 1H), 7.05 (t, J=7.6 Hz, 1H), 7.18-7.26 (m, 4H), 7.33 (t, J=7.6 Hz, 1H), 7.49-7.51 (m, 2H), 8.50(s, 1H).

IR(ATR); 3207,1705,1619,1471,1228,744 cm⁻¹.

EI-MS m/z; 352(M⁺).

### EXAMPLE 29

### Production of 1′-[2-{(naphtho-1-yl)oxy}acetyl]spiro(indole-3,4′-piperidin e)-2(1H)-one

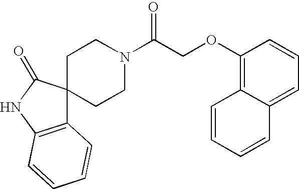

The reaction and treatment were conducted in a similar manner to process 2 of example 2 using 1-naphthol in place of ortho-cresol, and the title compound was obtained as a red-brown amorphous solid.

¹H-NMR (400 MHz, CDCl₃) δ; 1.74-1.85 (m, 4H), 3.80-3.87 (m, 1H), 3.98-4.03 (m, 1H), 4.07-4.13 (m, 1H), 4.27-4.32 (m, 1H), 4.94 (d, J=13.2 Hz, 1H), 5.01 (d, J=13.2 Hz, 1H), 6.86-7.00 (m, 4H), 7.18 (t, J=7.3 Hz, 0.25(s, 1H), 8.29 (d, J=7.3 Hz, 1H).

IR(ATR); 3236,1707,1647,1471,1230,772 cm⁻¹.

EI-MS m/z; 386(M⁺).

### EXAMPLE 30

### Production of 1′-[2-{(naphtho-2-yl)oxy}acetyl]spiro(indole-3,4′-pipetidin e)-2(1H)-one

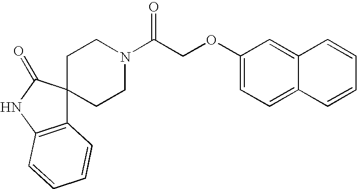

The reaction and treatment were conducted in a similar manner to process 2 of example 2 using 2-naphthol in place of ortho-cresol, and the title compound was obtained as a red-brown amorphous solid.

¹H-NMR (400 MHz, CDCl₃) δ; 1.75-1.92 (m, 4H), 3.79-3.86 (m, 1H), 3.93-3.97 (m, 1H), 4.05-4.11 (m, 1H), 4.27-4.32 (m, 1H), 4.87 (d, J=13. 1 Hz, 1H), 4.92 (d, J=13.1 Hz, 1H), 6.88 (d, J=7.7 Hz, 1H), 6.92-6.99 (m, 2H), 7.18-7.27 (m, 3H), 7.38 (t, J=7.7 Hz, 1H, 7.47 (t, J=7.7 Hz, 1H), 7.77-7.80 (m, 3H), 8.02 (s, 1H).

IR(ATR); 3213,1706,1630,1471,1215,749 cm⁻¹.

EI-MS m/z; 386(M⁺).

### EXAMPLE 31

### Production of 1′-{2-(2,6-difluorophenoxy)acetyl}spiro(indole-3,4′-piperid ine)-2(1H)-one

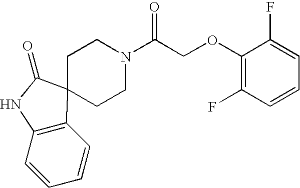

The reaction and treatment were conducted in a similar manner to process 2 of example 2 using 2,6-difluorophenol in place of ortho-cresol, and the title compound was obtained as a red-brown amorphous solid.

¹H-NMR (400 MHz, CDCl₃) δ; 1.89-2.04 (m, 4H), 3.84-3.90 (m, 2H), 4.09-4.15 (m, 1H), 4.24-4.28 (m, 1H), 4.83 (d, J=12.9 Hz, 1H), 4.94 (d, J=12.9 Hz, 1H), 6.89-7.02 (m, 4H), 7.06 (t, J=7.7 Hz, 1H), 7.,22-7.27 (m, 2H), 8.86 (s, 1H).

IR(ATR); 3237,1707,1647,1473,1009,750 cm⁻¹.

EI-MS m/z; 372(M⁺).

### EXAMPLE 32

### Production of 1′-{2-(2,6-dichlorophenoxy)acetyl}spiro(indole-3,4′-piperid ine)-2(1H)-one

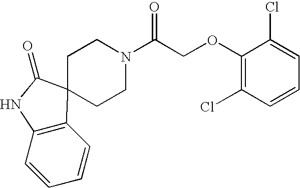

The reaction and treatment were conducted in a similar manner to process 2 of example 2 using 2,6-dichlorophenol in place of ortho-cresol, and the title compound was obtained as a red-brown amorphous solid.

¹H-NMR (400 MHz, CDCl₃) δ; 1.92-2.04 (m, 4H), 3.89-3.96 (m, 1H), 4.02-4.05 (m, 1H), 4.16-4.21 (m, 1H), 4.27-4.31 (m, 1H), 4.68 (d, J=12. 2 Hz, 1H), 4.84 (d, J=12.2 Hz, 1H), 6.95 (d, J=7.6 Hz, 1H), 7.02-7.07 (m, 2H), 7.22-7.26 (m, 2H), 7.32 (d, J=8.3 Hz, 2H), 8.76 (s, 1H).

IR(ATR); 3248,1707,1647,1471,1231,749 cm⁻¹.

EI-MS m/z; 404(M⁺).

### EXAMPLE 33

### Production of 1′-{2-(2,6-dimethoxyphenoxy)acetyl}spiro(indole-3,4′-piperi dine)-2(1H)-one

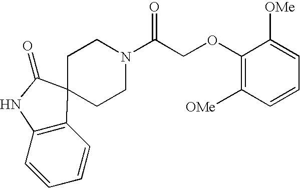

The reaction and treatment were conducted in a similar manner to process 2 of example 2 using 2,6-dimethoxyphenol in place of ortho-cresol, and the title compound was obtained as a red-brown amorphous solid.

¹H-NMR (400 MHz, CDCl₃) δ; 1.80-2.00 (m, 4H), 3.85 (s, 6H), 3.89-3.96 (m, 1H), 4.19-4.25 (m, 3H), 4.67 (s, 2H), 6.58 (d, J=8.3 Hz, 2H), 6.93 (d, J=7.8 Hz, 1H), 7.00-7.06 (m, 2H), 7.21-7.27 (m, 2H), 8.60 (s, 1H).

IR(ATR); 3208,1707,1620,1478,1110,749 cm⁻¹.

EI-MS m/z; 396(M⁺).

### EXAMPLE 34

### Production of 1′-{2-(2,6-dimethylphenoxy)acetyl}spiro(indole-3,4′-piperid ine)-2(1H)-one

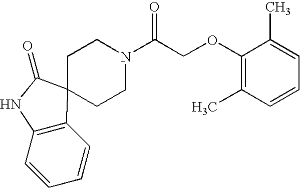

The reaction and treatment were conducted in a similar manner to process 2 of example 2 using 2,6-dimethylphenol in place of ortho-cresol, and the title compound was obtained as a red-brown amorphous solid.

¹H-NMR (400 MHz, CDCl₃) δ; 1.91-1.96 (m, 4H), 2.34 (s, 6H), 3.85-3.93 (m, 2H), 4.07-4.13 (m, 1H), 4.29-4.32 (m, 1H), 4.48 (d, J=12.7 Hz, 1 H), 4.61 (d, J=12.7 Hz, 1H), 6.90-6.97 (m, 2H), 7.02-7.08 (m, 3H), 7.23 (d, J=7.6 Hz, 2H), 7.73 (s, 1H).

### EXAMPLE 35

### Production of 1′-{2-(4-trifluoromethylphenoxy)acetyl}spiro(indole-3,4′-pi peridine)-2(1H)-one

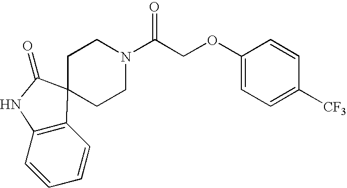

The reaction and treatment were conducted in a similar manner to process 2 of example 2 using 4-trifluoromethylphenol in place of ortho-cresol, and the title compound was obtained as a red-brown amorphous solid.

¹H-NMR (400 MHz, CDCl₃) δ; 1.76-1.93 (m, 4H), 3.80-3.88 (m, 2H), 4.04-4.10 (m, 1H), 4.24-4.28 (m, 1H), 4.81 (d, J=13.4 Hz, 1H), 4.89 (d, J=13.4 Hz, 1H), 6.91 (d, J=7.8 Hz, 1H), 7.03-7.12 (m, 4H), 7.20-7.25 (m, 1H), 7.59 (d, J=8.8 Hz, 2H), 8.42 (s, 1H).

IR(ATR); 3206,1707,1647,1329,1112,749 cm⁻¹.

EI-MS m/z; 404(M⁺).

### EXAMPLE 36

### Production of 1′-{2-(3-trifluoromethylphenoxy)acetyl}spiro(indole-3,4′-pi peridine)-2(1H)-one

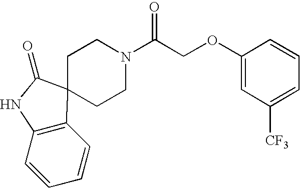

The reaction and treatment were conducted in a similar manner to process 2 of example 2 using 3-trifluoromethylphenol in place of ortho-cresol, and the title compound was obtained as a red-brown amorphous solid.

¹H-NMR (400 MHz, CDCl₃) δ; 1.78-1.95 (m, 4H), 3.83-3.88 (m, 2H), 4.05-4.12 (m, 1H), 4.24-4.29 (m, 1H), 4.80 (d, J=13.4 Hz, 1H), 4.86 (d, J=13.4 Hz, 1H), 6.92 (d, J=7.8 Hz, 1H), 7.02-7.11 (m, 2H), 7.19-7.29 (m, 4H), 7.44 (t, J=7.8 Hz, 1H), 8.58 (s, 1H).

IR(ATR); 3207,1706,1647,1472,1328,750 cm⁻¹.

EI-MS m/z; 404(M⁺).

### EXAMPLE 37

### Production of 1′-{2-(2,4-difluorophenoxy)acetyl}spiro(indole-3,4′-piperid ine)-2(1H)-one

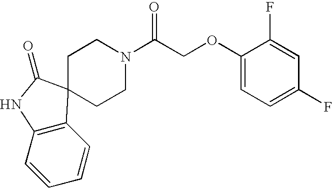

The reaction and treatment were conducted in a similar manner to process 2 of example 2 using 2,4-difluorophenol in place of ortho-cresol, and the title compound was obtained as a red-brown amorphous solid.

¹H-NMR (400 MHz, CDCl₃) δ; 1.81-1.92 (m, 4H), 3.81-3.90 (m, 2H), 4.05-4.12 (m, 1H), 4.23-4.27 (m, 1H), 4.78 (d, J=13.2 Hz, 1H), 4.85 (d, J=13.2 Hz, 1H), 6.80-6.93 (m, 3H), 7.03-7.15 (m, 3H), 7.22-7.26 (m, 1H), 8.47(s, 1H).

IR(ATR); 3212,1705,1647,1512,1207,749 cm⁻¹.

EI-MS m/z; 372(M⁺).

### EXAMPLE 38

### Production of 1′-{2-(2,4-dichlorophenoxy)acetyl}spiro(indole-3,4′-piperid ine)-2(1H)-one

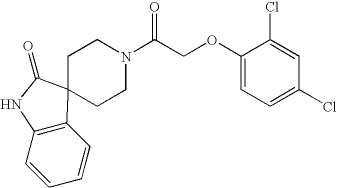

The reaction and treatment were conducted in a similar manner to process 2 of example 2 using 2,4-dichlorophenol in place of ortho-cresol, and the title compound was obtained as a red-brown amorphous solid.

¹H-NMR (400 MHz, CDCl₃) δ; 1.77-1.94 (m, 4H), 3.76-3.83(m, 1H), 3.92-3.96 (m, 1H), 4.05-4.12 (m, 1H), 4.25-4.29 (m, 1H), 4.79(d, J=13.2 Hz, 1H), 4.89 (d, J=13.2 Hz, 1H), 6.89 (d, J=7.6 Hz, 1H), 7.01-7.07 (m, 3H), 7.21-7.25 (m, 2H), 7.40 (d, J=2.4 Hz, 1H), 7.85 (s, 1H).

IR(ATR); 3234,1706,1647,1472,1229,750 cm⁻¹.

EI-MS m/z; 404(M⁺).

### EXAMPLE 39

### Production of 1′-{2-(2,3-difluorophenoxy)acetyl}spiro(indole-3,4′-piperid ine)-2(1H)-one

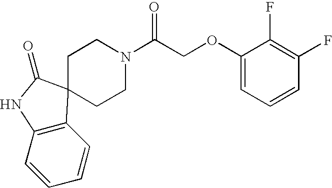

The reaction and treatment were conducted in a similar manner to process 2 of example 2 using 2,3-difluorophenol in place of ortho-cresol, and the title compound was obtained as a red-brown amorphous solid.

¹H-NMR (400 MHz, CDCl₃) δ; 1.80-1.93 (m, 4H), 3.81-3.90 (m, 2H), 4.06-4.13 (m, 1H), 4.24-4.28 (m, 1H), 4.83 (d, J=13.2 Hz, 1H), 4.90 (d, J=13.2 Hz, 1H), 6.83-6.93 (m, 3H), 7.01-7.07 (m, 2H), 7.13 (d, J=7.7 Hz, 1H), 7.23 (t, J=7.7 Hz, 1H), 8.53 (s, 1H).

IR(ATR); 3245,1706,1647,1481,1092,750 cm⁻¹.

EI-MS m/z; 372(M⁺).

### EXAMPLE 40

### Production of 1′-{2-(2,3-dichlorophenoxy)acetyl}spiro(indole-3,4′-piperid ine)-2(1H)-one

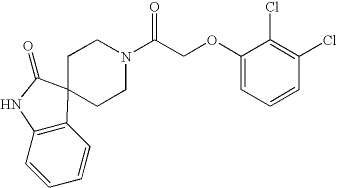

The reaction and treatment were conducted in a similar manner to process 2 of example 2 using 2,3-dichlorophenol in place of ortho-cresol, and the title compound was obtained as a red-brown amorphous solid.

¹H-NMR (400 MHz, CDCl₃) δ; 1.78-1.93 (m, 4H), 3.79-3.86 (m, 1H), 3.92-3.97 (m, 1H), 4.06-4.15 (m, 1H), 4.23-4.28 (m, 1H), 4.82 (d, J=13.2 Hz, 1H), 4.91 (d, J=13.2 Hz, 1H), 6.91 (d, J=7.8 Hz, 1H), 6.99-7.08 (m, 3H), 7.14-7.27 (m, 3H), 8.49 (s, 1H).

IR(ATR); 3246,1705,1647,1471,1231,749 cm⁻¹.

EI-MS m/z; 404(M⁺).

### EXAMPLE 41

### Production of 1′-{2-(4-bromo-2-fluorophenoxy)acetyl}spiro(indole-3,4′-pip eridine)-2(1H)-one

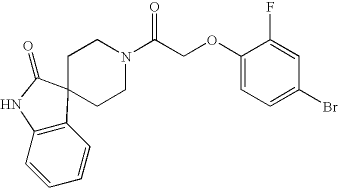

The reaction and treatment were conducted in a similar manner to process 2 of example 2 using 4-bromo-2-fluorophenol in place of ortho-cresol, and the title compound was obtained as a red-brown amorphous solid.

¹H-NMR (400 MHz, CDCl₃) δ; 1.78-1.94 (m, 4H), 3.79-3.88 (m, 2H), 4.04-4.11 (m, 1H), 4.23-4.27 (m, 1H), 4.79 (d, J=13.2 Hz, 1H), 4.87 (d, J=13.2 Hz, 1H), 6.91 (d, J=7.6 Hz, 1H), 6.99-7.12 (m, 3H), 7.21-7.28 (m, 3H), 8.39 (s, 1H).

IR(ATR); 3245,1706,1647,1498,1200,749 cm⁻¹.

EI-MS m/z; 432(M⁺).

### EXAMPLE 42

### Production of 1′-{2-(2-chloro-4-fluorophenoxy)acetyl}spiro(indole-3,4′-pi peridine)-2(1H)-one

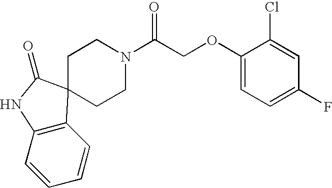

The reaction and treatment were conducted in a similar manner to process 2 of example 2 using 2-chloro-4-fluorophenol in place of ortho-cresol, and the title compound was obtained as a red-brown amorphous solid.

¹H-NMR (400 MHz, CDCl₃) δ; 1.78-1.93(m, 4H), 3.79-3.86 (m, 1H), 3.93-3.98 (m, 1H), 4.07-4.12 (m, 1H), 4.23-4.28 (m, 1H), 4.78 (d, J=13.3 Hz, 1H), 4.87 (d, J=13.3 Hz, 1H), 6.92 (d, J=7.8 Hz, 1H), 6.96-6.99 (m, 1H), 7.04-7.10 (m, 3H), 7.15 (dd, J=3.0, 7.8 Hz, 1H), 7.23 (dt, J=1.2, 7.8 Hz, 1H), 8.47 (s, 1H).

IR(ATR); 3306,1707,1639,1471,1194,761 cm⁻¹.

EI-MS m/z; 388(M⁺).

### EXAMPLE 43

### Production of

### 1′-{2-(4-chloro-2-fluorophenoxy)acetyl}spiro(indole-3,4′-pi peridine)-2(1H)-one

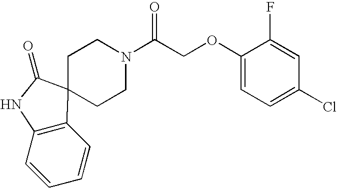

The reaction and treatment were conducted in a similar manner to process 2 of example 2 using 4-chloro-2-fluorophenol in place of ortho-cresol, and the title compound was obtained as a red-brown amorphous solid.

¹H-NMR (400 MHz, CDCl₃) δ; 1.76-1.95 (m, 4H), 3.79-3.89 (m, 2H), 4.05-4.11 (m, 1H), 4.23-4.27 (m, 1H), 4.79 (d, J=13.3 Hz, 1H), 4.87 (d, J=13.3 Hz, 1H), 6.92 (d, J=7.8 Hz, 1H), 7.03-7.14 (m, 5H), 7.23 (dt, J=1.3, 7.8 Hz, 1H), 8.40 (s, 1H).

IR(ATR); 3228,1706,1647,1500,1202,749 cm⁻¹.

EI-MS m/z; 388(M⁺).

### EXAMPLE 44

### Production of 1′-{2-(2-chloro-6-fluorophenoxy)acetyl}spiro(indole-3,4′-pi peridine)-2(1H)-one

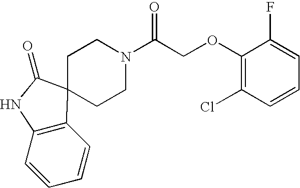

The reaction and treatment were conducted in a similar manner to process 2 of example 2 using 2-chloro-6-fluorophenol in place of ortho-cresol, and the title compound was obtained as a red-brown amorphous solid.

¹H-NMR (400 MHz, CDCl₃) δ; 1.77-1.90 (m, 4H), 3.78-3.84 (m, 1H), 3.91-3.96 (m, 1H), 4.05-4.11 (m, 1H), 4.23-4.28 (m, 1H), 4.77 (d, J=12. 4 Hz, 1H), 4.92 (d, J=12.4 Hz, 1H), 6.94 (d, J=7.3 Hz, 1H), 7.01-7.08 (m, 3H), 7.17-7.27 (m, 3H), 8.60 (s, 1H).

IR(ATR); 3247,1707,1647,1472,1227,750 cm⁻¹.

EI-MS m/z; 388(M⁺).

### EXAMPLE 45

### Production of 1′-{2-(4-bromo-2-chlorophenoxy)acetyl}spiro(indole-3,4′-pip eridine)-2(1H)-one

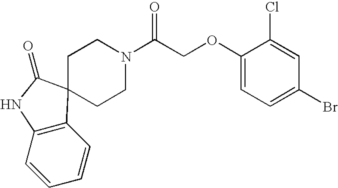

The reaction and treatment were conducted in a similar manner to process 2 of example 2 using 4-bromo-2-chlorophenol in place of ortho-cresol, and the title compound was obtained as a red-brown amorphous solid.

¹H-NMR (400 MHz, CDCl₃) δ; 1.91-2.04 (m, 4H), 3.85-3.97 (m, 2H), 4.12-4.19 (m, 1H), 4.26-4.31 (m, 1H), 4.80 (d, J=13.3 Hz, 1H), 4.89 (d, J=13.3 Hz, 1H), 6.91 (d, J=7.6 Hz, 1H), 6.97 (d, J=8.8 Hz, 1H), 7.03-7.06 (m, 2H), 7.21-7.25 (m, 1H), 7.36 (dd, J=2.2, 8 Hz, 1H), 7.53 (d, J=2.2 Hz, 1H), 8.38 (s, 1H).

IR(ATR); 3308,1708,1631,1470,1064,759 cm⁻¹.

EI-MS m/z; 448(M⁺).

### EXAMPLE 46

### Production of 1′-{2-(2-bromo-4-chlorophenoxy)acetyl}spiro(indole-3,4′-pip eridine)-2(1H)-one

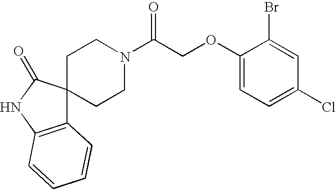

The reaction and treatment were conducted in a similar manner to process 2 of example 2 using 2-bromo-4-chlorophenol in place of ortho-cresol, and the title compound was obtained as a red-brown amorphous solid.

¹H-NMR (400 MHz, CDCl₃) δ; 1.76-1.94 (m, 4H), 3.76-3.83 (m, 1H), 3.94-3.97 (m, 1H), 4.05-4.12 (m, 1H), 4.24-4.28 (m, 1H), 4.79 (d, J=13.4 Hz, 1H), 4.89 (d, J=13.4 Hz, 1H), 6.90 (d, J=7.6 Hz, 1H), 7.00 (d, J=8.8 Hz, 1H), 7.04-7.06 (m, 2H), 7.21-7.29 (m, 2H), 7.56-7.57 (m, 1H), 8.02 (s, 1H).

IR(ATR); 3303,1708,1630,1470,1050,759 cm⁻¹.

EI-MS m/z; 448(M⁺).

### EXAMPLE 47

### Production of 1′-{2-(2-fluorophenoxy)acetyl}spiro(indole-3,4′-piperidine)-2(1H)-one

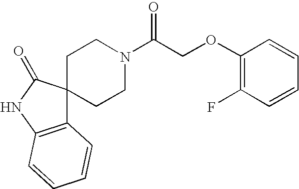

The reaction and treatment were conducted in a similar manner to process 2 of example 2 using 2-fluorophenol in place of ortho-cresol, and the title compound was obtained as a red-brown amorphous solid.

¹H-NMR (400 MHz, CDCl₃) δ; 1.90(m, 4H), 3.84 (m, 1H), 3.93 (m, 1H), 4.10(m, 1H), 4.26 (m, 1H), 4.80 (d, J=13.4 Hz, 1H), 4.86 (d, J=13.4 Hz, 1 H), 7.05(m, 7H), 7.21 (m, 1H), 8.54 (s, 1H).

IR(ATR); 3236,1706,1647,1505,1259,746 cm⁻¹.

EI-MS m/z; 354(M⁺).

### EXAMPLE 48

### Production of 1′-{2-(2-chlorophenoxy)acetyl}spiro(indole-3,4′-piperidine)-2(1H)-one

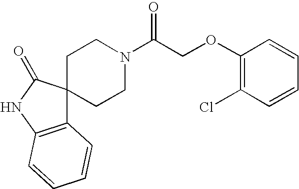

The reaction and treatment were conducted in a similar manner to process 2 of example 2 using 2-chlorophenol in place of ortho-cresol, and the title compound was obtained as a red-brown amorphous solid.

¹H-NMR (400 MHz, CDCl₃) δ; 1.85(m, 4H), 3.82 (m, 1H), 4.00 (m, 1H), 4.09 (m, 1H), 4.27 (m, 1H), 4.80 (d, J=13.2 Hz, 1H), 4.90 (d, J=13.2 Hz, 1 H), 6.91(d, J=7.6 Hz, 1H), 6.98 (m, 1H), 7.04 (m, 2H), 7.08 (d, J=8.4 Hz, 1H), 7.23(m, 2H), 7.39 (dd, J=1.5, 7.8 Hz, 1H), 8.49 (s, 1H).

IR(ATR); 3313,1707,1635,1484,1246,741 cm⁻¹.

EI-MS m/z; 370(M⁺).

### EXAMPLE 49

### Production of 1′-{2-(2-bromophenoxy)acetyl}spiro(indole-3,4′-piperidine)-2(1H-one

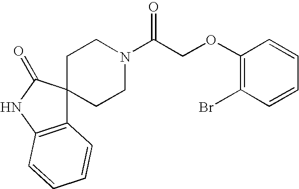

The reaction and treatment were conducted in a similar manner to process 2 of example 2 using 2-bromophenol in place of ortho-cresol, and the title compound was obtained as a red-brown amorphous solid.

¹H-NMR (400 MHz, CDCl₃) δ; 1.84(m, 4H), 3.82 (m, 1H), 4.04 (m, 1H), 4.11 (m, 1H), 4.27 (m, 1H), 4.82 (d, J=13.2 Hz, 1H), 4.93 (d, J=13.2 Hz, 1 H) 6.92 (m, 2H), 7.06 (m, 3H), 7.22 (m, 1H), 7.31 (m, 1H), 7.56 (dd, J=1.7, 8.0 Hz, 1H), 8.41(s, 1H).

IR(ATR); 3312,1706,1636,1470,1246,741 cm⁻¹.

EI-MS m/z; 414(M⁺).

### EXAMPLE 50

### Production of 1′-{2-(2-ethylphenoxy)acetyl}spiro(indole-3,4′-piperidine)-2(1H)-one

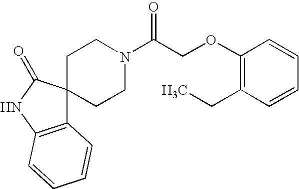

The reaction and treatment were conducted in a similar manner to process 2 of example 2 using 2-ethylphenol in place of ortho-cresol, and the title compound was obtained as a red-brown amorphous solid.

¹H-NMR (400 MHz, CDCl₃) δ; 1.22 (t, J=7.6 Hz, 2H), 1.82 (m, 4H), 2.69 (m, 2H), 3.81 (m, 1H), 3.99 (m, 1H), 4.07 (m, 1H), 4.31 (m, 1H), 4.73 (d, J=12.9 Hz, 1H), 4.84(d., J=12.9 Hz, 1H), 6.87 (d, J=7.8 Hz, 1H), 6.95 (m, 2H), 7.04 (m, 2H), 7.22 (m, 3H), 7.46 (s, 1H).

### EXAMPLE 51

### Production of 1′-{2-(2-propylphenoxy)acetyl}spiro(indole-3,4′-piperidine)-2(1H)-one

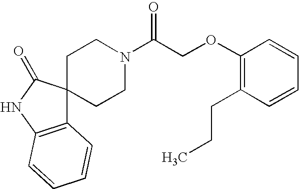

The reaction and treatment were conducted in a similar manner to process 2 of example 2 using 2-propylphenol in place of ortho-cresol., and the title compound was obtained as a red-brown amorphous solid.

¹H-NMR (400 MHz, CDCl₃) δ; 0.95(t, J=7.3 Hz, 3H), 1.70 (m, 2H) 1.86 (m, 4H), 2.64 (m, 2H), 3.79 (m, 1H), 3.98 (m, 1H), 4.06 (m, 1H), 4.30 (m, 1H), 4.73 (d, J=12.9 Hz, 1H), 4.79 (d, J=12.9 Hz, 1H), 6.87 (d, J=7.8 Hz, 1H), 6.95 (m, 2H), 7.04 (m, 2H), 7.20 (m, 3H), 7.38 (s, 1H).

### EXAMPLE 52

### Production of 1′-{2-(2-t-butylphenoxy)acetyl}spiro(indole-3,4′-piperidine)-2(1H)-one

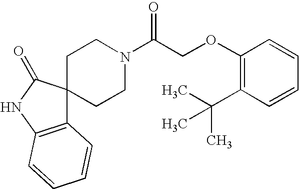

The reaction and treatment were conducted in a similar manner to process 2 of example 2 using 2-t-butylphenol in place of ortho-cresol, and the title compound was obtained as a red-brown amorphous solid.

¹H-NMR (400 MHz, CDCl₃) δ; 1.48(s, 9H), 1.88 (m, 4H), 3.83 (m, 1H), 3.95(m, 1H), 4.10 (m, 1H), 4.32 (m, 1H), 4.72 (d, J=12.9 Hz, 1H), 4.83 (d, J=12.9 Hz, 1H), 6.87 (d, J=7.9 Hz, 1H), 6.97 (m, 2H), 7.04 (t, J=7.9 Hz, 1H), 7.09 (d, J=7.9 Hz, 1H), 7.24 (m, 4H).

### EXAMPLE 53

### Production of 1′-{2-(2-phenylphenoxy)acetyl}spiro(indole-3,4′-piperidine)-2(1H)-one

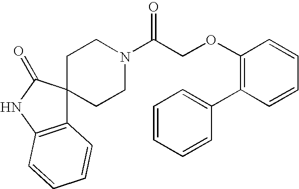

The reaction and treatment were conducted in a similar manner to process 2 of example 2 using 2-phenylphenol in place of ortho-cresol, and the title compound was obtained as a red-brown amorphous solid.

¹H-NMR (400 MHz, CDCl₃) δ; 1.45(m, 1H), 1.55 (m, 1H), 1.72 (m, 2H), 3.71 (m, 2H), 3.85 (m, 1H), 4.26 (m, 1H), 4.70 (d, J=13.2 Hz, 1H), 4.76 (d, J=13.2 Hz, 1H), 6.86 (d, J=7.8 Hz, 1H), 6.92 (d, J=7.8 Hz, 1H), 7.08 (m, 3H), 7.30 (m, 6H), 7.50 (m, 2H), 7.75 (s, 1H).

IR(ATR); 3231,1705,1638,1472,1216,749 cm⁻¹.

EI-MS m/z; 412(M⁺).

### EXAMPLE 54

### Production of 1′-{2-(3-fluorophenoxy)acetyl}spiro(indole-3,4′-piperidine)-2(1H)-one

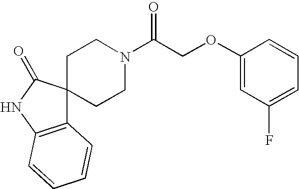

The reaction and treatment were conducted in a similar manner to process 2 of example 2 using 3-fluorophenol in place of ortho-cresol, and the title compound was obtained as a red-brown amorphous solid.

¹H-NMR(270 MHz, CDCl₃) δ; 1.84(m, 4H), 3.85 (m, 2H), 4.06 (m, 1H), 4.24 (m, 1H), 4.72 (d, J=13.2 Hz, 1H), 4.82 (d, J=13.2 Hz, 1H), 6.73 (m, 3 H), 6.92(d, J=8.1 Hz, 1H), 7.07 (m, 2H), 7.24 (m, 2H), 8.50 (s, 1H).

IR(ATR); 3227,1709,1642,1471,1146,753 cm⁻¹.

EI-MS m/z; 354(M⁺).

### EXAMPLE 55

### Production of 1′-{2-(3-bromophenoxy)acetyl}spiro(indole-3,4′-piperidine)-2(1H)-one

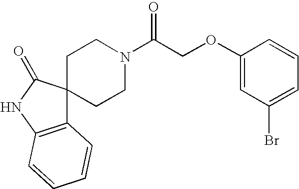

The reaction and treatment were conducted in a similar manner to process 2 of example 2 using 3-bromophenol in place of ortho-cresol, and the title compound was obtained as a red-brown amorphous solid.

¹H-NMR (270 MHz, CDCl₃) δ; 1.90(m, 4H), 3.85 (m, 2H), 4.06 (m, 1H), 4.26 (m, 1H), 4.73 (d, J=13.2 Hz, 1H), 4.79 (d, J=13.2 Hz, 1H), 6.93 (m, 2 H), 7.14 (m, 5H), 7.23 (m, 1H), 8.50 (s, 1H).

IR(ATR); 3219,1706,1644,1473,1225,750 cm⁻¹.

EI-MS m/z; 414(M⁺).

### EXAMPLE 56

### Production of 1′-{2-(2,5-difluorophenoxy)acetyl}spiro(indole-3,4′-piperid ine)-2(1H)-one

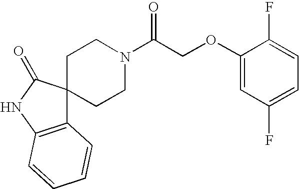

The reaction and treatment were conducted in a similar manner to process 2 of example 2 using 2,5-difluorophenol in place of ortho-cresol, and the title compound was obtained as a red-brown amorphous solid.

¹H-NMR (270 MHz, CDCl₃) δ; 1.90(m, 4H), 3.85 (m, 2H) 4.09 (m, 1H), 4.27 (m, 1H), 4.76 (d, J=13.5 Hz, 1H), 4.89 (d, J=13.5 Hz, 1H), 6.67 (m, 1 H), 6.85 (m, 5H), 6.93 (d, J=7.6 Hz, 1H), 7.10 (m, 2H), 7.24 (m, 1H), 8.74 (s, 1H).

IR(ATR); 3248,1705,1648,1472,1203,749 cm⁻¹.

EI-MS m/z; 372(M⁺).

### EXAMPLE 57

### Production of 1′-{2-(3,5-dichlorophenoxy)acetyl}spiro(indole-3,4′-piperid ine)-2(1H)-one

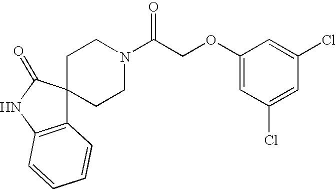

The reaction and treatment were conducted in a similar manner to process 2 of example 2 using 3,5-dichlorophenol in place of ortho-cresol, and the title compound was obtained as a red-brown amorphous solid.

¹H-NMR (270 MHz, CDCl₃) δ; 1.90(m, 4H), 3.80 (m, 2H), 4.07 (m, 1H), 4.26 (m, 1H), 4.72 (d, J=13.5 Hz, 1H), 4.80 (d, J=13.5 Hz, 1H), 6.89 (d, J=2.2 Hz, 2H), 6.93 (d, J=7.6 Hz, 1H), 7.01 (t, J=2.2 Hz, 1H), 7.06 (dt, J=1.1, 7.6 Hz, 1H), 7.16 (d, J=7.6 Hz, 1H), 7.25 (dt, J=1.1, 7.6 Hz, 1H), 8. 76(s, 1H).

IR(ATR); 3219,1706,1644,1473,1225,750 cm⁻¹.

EI-MS m/z; 404(M⁺).

### EXAMPLE 58

### Production of 1′-{2-(4-chloro-3-fluorophenoxy)acetyl}spiro(indole-3,4′-pi peridine)-2(1H)-one

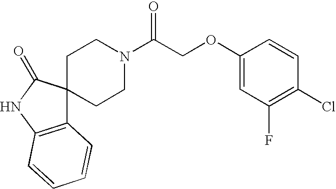

The reaction and treatment were conducted in a similar manner to process 2 of example 2 using 4-chloro-3-fluorophenol in place of ortho-cresol, and the title compound was obtained as a red-brown amorphous solid.

¹H-NMR (270 MHz, CDCl₃) δ; 1.86(m, 4H), 3.81 (m, 1H), 3.96 (m, 1H), 4.08 (m, 1H), 4.25 (m, 1H), 4.78 (d, J=13.2 Hz, 1H), 4.68 (d, J=13.2 Hz, 1 H), 6.89 (d, J=8.1 Hz, 1H), 7.04 (m, 4H), 7.24 (m, 1H), 7.35 (m, 1H), 8.33 (s, 1H).

IR(ATR); 3210,1707,1638,1470,1191,760 cm⁻¹.

EI-MS m/z; 388(M⁺).

### EXAMPLE 59

### Production of 1′-{2-(2-bromo-4-fluorophenoxy)acetyl}spiro(indole-3,4′-pip eridine)-2(1H)-one

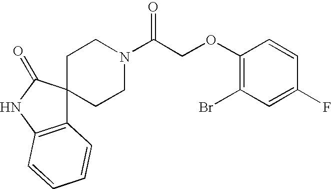

The reaction and treatment were conducted in a similar manner to process 2 of example 2 using 2-bromo-4-fluorophenol in place of ortho-cresol, and the title compound was obtained as a red-brown amorphous solid.

¹H-NMR (270 MHz, CDCl₃) δ; 1.85(m, 4H), 3.83 (m, 2H), 4.08 (m, 1H), 4.24 (m, 1H), 4.72 (d, J=13.5 Hz, 1H), 4.80 (d, J=13.5 Hz, 1H), 6.73 (m, 1 H), 6.81 (dd, J=3.0, 10.8 Hz, 1H), 6.93 (d, J=7.6 Hz, 1H), 7.05 (t, J=7.6 Hz, 1H), 7.12 (d, J=7.6 Hz, 1H), 7.22 (dd, J=1.6, 7.6 Hz, 1H), 7.34 (t, J=7.6 Hz, 1H), 8.68 (s, 1H).

IR(ATR); 3299,1707,1637,1471,1170,759 cm⁻¹.

EI-MS m/z; 432(M⁺).

### EXAMPLE 60

### Production of 1′-{2-(2-bromo-5-fluorophenoxy)acetyl}spiro(indole-3,4′-pip eridine)-2(1H)-one

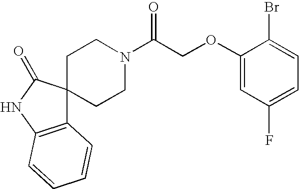

The reaction and treatment were conducted in a similar manner to process 2 of example 2 using 2-bromo-5-fluorophenol in place of ortho-cresol, and the title compound was obtained as a red-brown amorphous solid.

¹H-NMR (400 MHz, CDCl₃) δ; 1.87(m, 4H), 3.84 (m, 1H), 3.93 (m, 1H), 4.12 (m, 1H), 4.27 (m, 1H), 4.79 (d, J=13.2 Hz, 1H), 4.87 (d, J=13.2 Hz, 1 H), 6.66 (m, 1H), 6.82 (dd, J=2.7, 10.2 Hz, 1H), 6.91 (d, J=7.8 Hz, 1H), 7.04 (t, J=7.8 Hz, 1H), 7.09 (d, J=6.1, 1H), 7.23(d, J=7.8 Hz, 1H), 7.51 (dd, J=6.1, 8.8 Hz, 1H), 8.55 (s, 1H).

IR(ATR); 3238,1704,1648,1472,1167,749 cm⁻¹.

EI-MS m/z; 432(M⁺).

### EXAMPLE 61

### Production of 1′-{2-(2-fluoro-5-trifluoromethylphenoxy)acetyl}spiro(indol e-3,4′-piperidine)-2(1H)-one

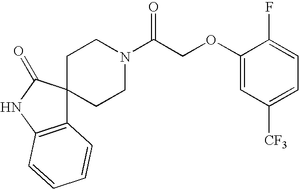

The reaction and treatment were conducted in a similar manner to process 2 of example 2 using 2-fluoro-5-trifluoromethylphenol in place of ortho-cresol, and the title compound was obtained as a red-brown amorphous solid.

¹H-NMR (400 MHz, CDCl₃) δ; 1.88(m, 2H), 1.94 (m, 2H), 3.85 (m, 2H), 4.10 (m, 1H), 4.28 (m, 1H), 4.84 (d, J=13.2 Hz, 1H), 4.93 (d, J=13.2 Hz, 1 H), 6.92(d, J=7.8 Hz, 1H), 7.06 (t, J=7.8 Hz, 1H), 7.14-7.30 (m, 4H), 7.32 (d, J=7.8 Hz, 1H), 8.51 (s, 1H).

IR(ATR); 3258,1707,1648,1472,1121,750 cm⁻¹.

EI-MS m/z; 422(M⁺).

### EXAMPLE 62

### Production of 1′-{2-(2-fluorophenylthio)acetyl}spiro(indole-3,4′-piperidi ne)-2(1H)-one

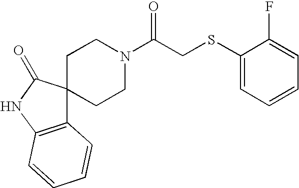

The reaction and treatment were conducted in a similar manner to process 2 of example 2 using 2-fluorothiophenol in place of ortho-cresol, and the title compound was obtained as a pale-yellow amorphous solid.

¹H-NMR (400 MHz, CDCl₃) δ; 1.80-2.00 (m, 4H), 3.70-3.87 (m, 4H), 4.02-4.09 (m, 1H), 4.18-4.23 (m, 1H), 6.92 (d, J=7.6 Hz, 1H), 7.04-7.16 (m, 3H), 7.21-7.32 (m, 3H), 7.60 (t, J=7.6 Hz, 1H), 8.55 (s, 1H).

IR(ATR); 3235,1705,1620,1472,1225,749 cm⁻¹.

EI-MS m/z; 370(M⁺).

### EXAMPLE 63

### Production of 1-{(2-(2-chlorophenylthio)acetyl}spiro(indole-3,4′-piperidi ne)-2(1H)-one

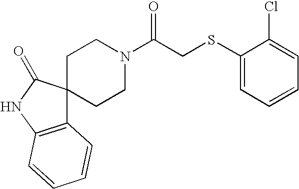

The reaction and treatment were conducted in a similar manner to process 2 of example 2 using 2-chlorothiophenol in place of ortho-cresol, and the title compound was obtained as a pale-yellow amorphous solid.

¹H-NMR (400 MHz, CDCl₃) δ; 1.78-1.95 (m, 4H), 3.77-3.91 (m, 4H) 4.04-4.11 (m, 1H), 4.20-4.24 (m, 1H), 6.92 (d, J=7.6 Hz, 1H), 7.05 (t, J=7.6 Hz, 1H), 7.17-71.30 (m, 4H), 7.40(t, J=7.8 Hz, 1H), 7.63 (d, J=7.8 Hz 1H), 8.40 (s, 1H).

IR(ATR); 3234,1706,1619,1471,1229,747 cm⁻¹.

EI-MS m/z; 386(M⁺).

### EXAMPLE 64

### Production of 1′-{2-(2-methoxyphenylthio)acetyl}spiro(indole-3,4′-piperid ine)-2(1H)-one

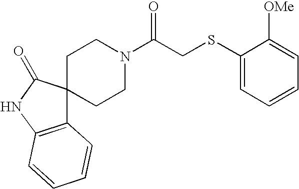

The reaction and treatment were conducted in a similar manner to process 2 of example 2 using 2-methoxythiophenol in place of ortho-cresol, and the title compound was obtained as a pale-yellow amorphous solid.

¹H-NMR (400 MHz, CDCl₃) δ; 1.77-1.93 (m, 4H), 3.76-3.87 (m, 4H) 3.91 (s, 3H), 4.02-4.08 (m, 1H), 4.15-4.21 (m, 1H), 6.88-6.93 (m, 2H), 6.96 (t, J=7.6 Hz, 1H), 7.05 (t, J=7.6 Hz, 1H), 7.19-7.27 (m, 3H), 7.54 (d, J=7.6 Hz, 1H), 8.58 (s, 1H).

IR(ATR); 3234,1706,1619,1472,1244,748 cm⁻¹.

EI-MS m/z; 382[M+].

### EXAMPLE 65

### Production of 1′-{2-(3-fluorophenylthio)acetyl}spiro(indole-3,4′-piperidi ne)-2(1H)-one

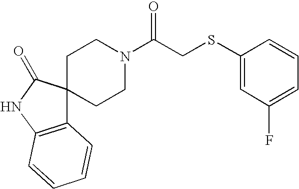

The reaction and treatment were conducted in a similar manner to process 2 of example 2 using 3-fluorothiophenol in place of ortho-cresol, and the title compound was obtained as a pale-yellow amorphous solid.

¹H-NMR (400 MHz, CDCl₃) δ; 1.80-1.94 (m, 4H), 3.70-3.91 (m, 4H), 4.03-4.10 (m, 1H), 4.21-4.24 (m, 1H), 6.93 (d, J=7.6 Hz, 2H), 7.06 (t, J=7.6 Hz, 1H), 7.19-7.32(m, 5H), 8.60 (s, 1H).

IR(ATR); 3235,1706,1619,1472,1219,749 cm⁻¹.

EI-MS m/z; 370(M⁺).

### EXAMPLE 66

### Production of 1′-{2-(3-chlorophenylthio)acetyl}spiro(indole-3,4′-piperidi ne)-2(1H)-one

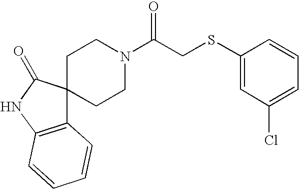

The reaction and treatment were conducted in a similar manner to process 2 of example 2 using 3-chlorothiophenol in place of ortho-cresol, and the title compound was obtained as a pale-yellow amorphous solid.

¹H-NMR (400 MHz, CDCl₃) δ; 1.78-1.94 (m, 4H), 3.70-3.90 (m, 4H) 4.02-4.09 (m, 1H), 4.20-4.26 (m, 1H), 6.93(d, J=7.6 Hz, 1H), 7.06 (t, J=7.6 Hz, 1H), 7.19-7.28 (m, 4H), 7.37-7.39 (m, 1H), 7.46-7.47 (m, 1H), 8.50 (s, 1H).

IR(ATR); 3200,1705,1619,1471,1229,749 cm⁻¹.

EI-MS m/z; 386(M⁺).

### EXAMPLE 67

### Production of 1′-{2-(3-methoxyphenylthio)acetyl}spiro(indole-3,4′-piperid ine)-2(1H)-one

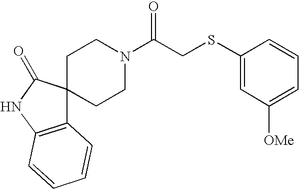

The reaction and treatment were conducted in a similar manner to process 2 of example 2 using 3-methoxythiophenol in place of ortho-cresol, and the title compound was obtained as a pale-yellow amorphous solid.

¹H-NMR (400 MHz, CDCl₃) δ; 1.78-1.91 (m, 4H), 3.70-3.86 (m, 4H) 3.81 (s, 3H), 4.00-4.07 (m, 1H), 4.20-4.25 (m, 1H), 6.77-6.79 (m, 1H), 6.92 (d, J=7.6 Hz, 1H), 7.03-7.07 (m, 3H), 7.18-7.26 (m, 3H), 8.59 (s, 1H

IR(ATR); 3227,1706,1620,1471,1230,749 cm⁻¹.

EI-MS m/z; 382(M⁺).

### EXAMPLE 68

### Production of 1′-{2-(4-fluorophenylthio)acetyl}spiro(indole-3,4′-piperidi ne)-2(1H)-one

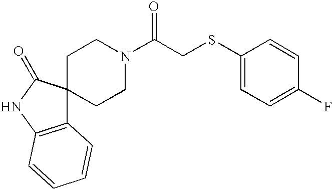

The reaction and treatment were conducted in a similar manner to process 2 of example 2 using 4-fluorothiophenol in place of ortho-cresol, and the title compound was obtained as a pale-yellow amorphous solid.

¹H-NMR (400 MHz, CDCl₃) δ; 1.78-1.96 (m, 4H), 3.68-3.88 (m, 4H), 4.00-4.07 (m, 1H), 4.16-4.21 (m, 1H), 6.93 (d, J=7.6 Hz, 1H), 7.02-7.08 (m, 3H), 7.19-7.27 (m, 2H), 7.50-7.54 (m, 2H), 8.59 (s, 1H).

IR(ATR); 3217,1705,1620,1471,1227,750 cm⁻¹.

EI-MS m/z; 370(M⁺).

### EXAMPLE 69

### Production of 1′-{2-(2,6-dichlorophenylthio)acetyl}spiro(indole-3,4′-pipe ridine)-2(1H)-one

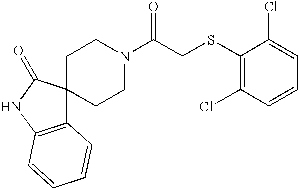

The reaction and treatment were conducted in a similar manner to process 2 of example 2 using 2,6-dichlorothiophenol in place of ortho-cresol, and the title compound was obtained as a pale-yellow amorphous solid.

¹H-NMR (400 MHz, CDCl₃) δ; 1.86-2.01(m, 4H), 3.69-3.86 (m, 4H) 4.03-4.10 (m, 1H), 4.15-4.20 (m, 1H), 6.93 (d, J=7.6 Hz, 1H), 7.06 (t, J=7.6 Hz, 1H), 7.20-7.26 (m, 3H), 7.41 (d, J=8.1 Hz, 2H), 8.63 (s, 1H).

IR(ATR); 3242,1706,1620,1471,1186,749 cm⁻¹.

EI-MS m/z; 420(M⁺).

### EXAMPLE 70

### Production of 1′-{2-(2-trifluoromethylphenylthio)acetyl}spiro(indole-3,4′-piperidine)-2(1H)-one

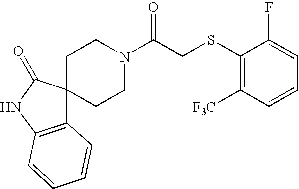

The reaction and treatment were conducted in a similar manner to process 2 of example 2 using 2-trifluoromethylthiophenol in place of ortho-cresol, and the title compound was obtained as a pale-yellow amorphous solid.

¹H-NMR (400 MHz, DMSO-d₆) δ; 1.65-1.83 (m, 4H), 3.77 (m, 2H) 3.90 (m, 2H), 4.18 (d, J=13.6 Hz, 1H), 4.26 (d, J=13.6 Hz, 1H), 6.86 (d, J=7.6 Hz, 1H), 6.97 (t, J=7.6 Hz, 1H), 7.20 (d, J=7.6 Hz, 1H), 7.39 (m, 2H), 7.6 4(m, 1H), 7.74 (t, J=8.6 Hz, 2H), 10.45 (s, 1H).

IR(ATR); 3256,1704,1630,1470,1118,761 cm⁻¹.

EI-MS m/z; 420(M⁺).

### EXAMPLE 71

### Production of 1′-{2-(2,6-dibromophenoxy)acetyl}spiro(indole-3,4′-piperidi ne)-2(1H)-one

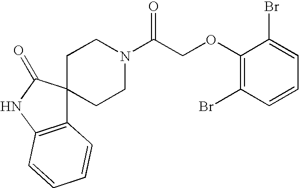

The reaction and treatment were conducted in a similar manner to process 2 of example 2 using 2,6-dibromophenol in place of ortho-cresol, and the title compound was obtained as a white amorphous solid.

¹H-NMR (400 MHz, CDCl₃) δ; 1.94-1.99 (m, 4H), 3.88-3.94 (m, 4H) 4.04-4.22 (m, 2H), 4.29-4.33 (m, 1H), 4.67 (d, J=12.0 Hz, 1H), 4.85 (d, J=12.0 Hz, 1H), 6.90-6.93 (m, 2H), 7.06 (t, J=7.6 Hz, 1H), 7.22-7.26 (m, 2H), 7.53 (d, J=1.8 Hz, 2H), 8.14 (s, 1H).

IR(ATR); 3209,2926,1708,1635,1471,1433,1230,749 cm⁻¹.

EI-MS m/z; 414(M⁺−80).

### EXAMPLE 72

### Production of 1′-{2-(phenylamino)acetyl}spiro(indole-3,4′-piperidine)-2(1H)-one

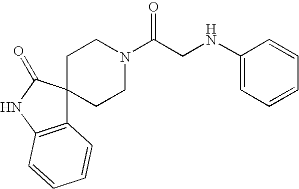

### Process 1

### Production of 1′-[2-{N-(2-nitrobenzenesulfonyl)-N-phenylamino}acetyl]spir o(indole-3,4′-piperidine)-2(1H)-one

The reaction and treatment were conducted in a similar manner to process 2 of example 2 using N-(2-nitrobenzenesulfonyl)aniline in place of ortho-cresol, and the title compound was obtained as white crystalline powder.

¹H-NMR (400 MHz, CDCl₃) δ; 1.79-1.99(m, 4H), 3.72 (m, 2H), 4.02 (m, 1H), 4.15 (m, 1H), 4.58 (d, J=16.9 Hz, 1H), 4.88(d, J=16.9 Hz, 1H), 6.91 (d, J=8.3 Hz, 1H), 7.06 (t, J=7.3 Hz, 1H), 7.22 (t, J=6.8 Hz, 2H), 7.32-7.37 (m, 3H), 7.45-7.54 (m, 3H), 7.63-7.69 (m, 3H), 8.48 (s, 1H).

### Process 2

### Production of 1′-{2-(phenylamino) acetyl}spiro(indole-3,4′-piperidine)-2(1H)-one

1′-{2-(phenylamino)acetyl}spiro(indole-3,4′-piperidine)-2(1H)-one was produced by the method described below. 1′-[2-{N-(2′-nitrobenzenesulfonyl)-N-phenylamino}acetyl]spiro(indole-3,4′-piperidine)-2(1H)-one (10.3 mg, 0.02 mmol) was dissolved in acetonitrile (1 mL), and potassium carbonate (5.5 mg, 0.04 mmol) and thiophenol (3 mg, 0.03 mmol) were added thereto. The mixture was stirred at room temperature for 1 hour. The reaction solution was added with water and extracted with diethyl ether. The organic layer was dried with anhydrous sodium sulfate, and the resultant residue was purified by preparative thin-layer chromatography (chloroform:ethyl acetate=1:2) and 6.2 mg (92.5%) of 1′-{2-(phenylamino)acetyl}spiro(indole-3,4′-piperidine)-2(1H)-one was obtained as a pale-yellow amorphous solid.

¹H-NMR (400 MHz, CDCl₃) δ; 1.91(m, 4H), 3.70 (m, 1H), 3.88 (m, 1H), 3.97 (s, 2H), 4.04 (m, 1H), 4.32 (m, 1H), 4.98 (s, 1H), 6.66 (d, J=7.6 Hz, 2H), 6.74 (t, J=7.6 Hz, 1H), 6.91(d, J=7.6 Hz, 1H), 7.06 (t, J=7.6 Hz, 1H), 7.18-7.26 (m, 4H), 7.94 (s, 1H).

IR(ATR); 3212,1698,1632,1472,1185,743 cm⁻¹.

EI-MS m/z; 335(M⁺).

### EXAMPLE 73

### Production of 1′-{8-(2-trifluoromethylphenoxy)octanoyl}spiro(indole-3,4′-piperidine)-2(1H)-one

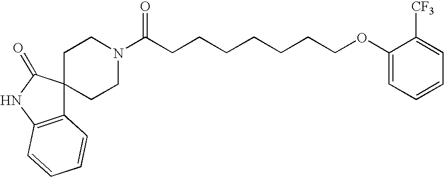

### Process 1

### Production of 1′-(8-bromooctanoyl) spiro(indole-3,4′-piperidine)-2(1H)-one

1′-(8-bromooctanoyl)spiro(indole-3,4′-piperidine)-2(1H)-one was produced by the method described below.

Diisopropylethylamine (95.6 mg, 0.740 mmol) and PyBOP (92.6 mg, 0.178 mmol) was added sequentially at room temperature to a methylene chloride solution (2 mL) of spiro(indole-3,4′-piperidine)-2(1H)-one (30.0 mg, 0.148 mmol) and 8-bromooctanoic acid (39.7 mg, 0.178 mmol). The mixture was stirred at the same temperature for 5.5 hours. The reaction solution was added with water and extracted with chloroform. The organic layer was dried with anhydrous sodium sulfate, followed by a vacuum concentration. The resultant residue was purified by silica-gel chromatography (hexane:ethyl acetate=2:1) and 1′-(8-bromooctanoyl) spiro(indole-3,4′-piperidine)-2(1H)-one (42.1 mg, 69.8%) was obtained as a white amorphous solid.

¹H-NMR (400 MHz, CDCl₃) δ; 1.38-1.47 (m, 6H), 1.65-1.72 (m, 2H), 1 (m, 1H), 3.80-3.87 (m, 1H), 3.97-4.03 (m, 1H), 4.17-4.23 (m, 1H), 6.90 (d, J=7.3 Hz, 1H), 7.06 (t, J=7.6 Hz, 1H), 7.22-7.26 (m, 2H), 7.65 (s, 1H).

### Process 2

### Production of 1′-{8-(2-trifluoromethylphenoxy)octanoyl}spiro(indole-3,4′-piperidine)-2(1H)-one

The reaction and treatment were conducted in a similar manner to process 2 of example 2 using 1′-(8-bromooctanoyl)spiro(indole-3,4-piperidine)-2(1H)-one in place of 1′-(2-bromoacetyl)spiro(indole-3,4′-piperidine)-2(1H)-one, as well as 2-trifluoromethylphenol in place of ortho-cresol, and the title compound was obtained as a white amorphous solid.

¹H-NMR (400 MHz, CDCl₃) δ; 1.26-1.91(m, 14H), 2.41(t, J=7.6 Hz, 2H), 3.66-3.79 (m, 1H), 3.82-3.89 (m, 1H), 3.95-4.10 (m, 3H), 4.15-4.2 5(m, 1H), 6.92 (d, J=8.0 Hz, 1H), 6.95-6.99 (m, 2H), 7.05 (t, J=7.6 Hz, 1H), 7.22-7.27 (m, 2H), 7.46 (t, J=7.8 Hz, 1H), 7.55 (d, J=7.8 Hz, 1H), 8.05 (s, 1H).

EI-MS m/z; 488(M⁺).

### EXAMPLE 74

### Production of 1′-{2-(2-trifluoromethylphenylsulfinyl)acetyl}spiro(indole-3,4′-piperidine)-2(1H)-one

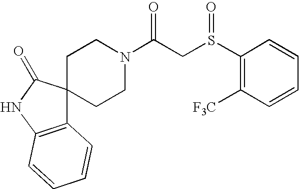

1′-{2-(2-trifluoromethylphenylsulfinyl)acetyl}spiro(in dole-3,4′-piperidine)-2(1H)-one was produced by the method described below.

To a methylene chloride solution (3 mL) of 1′-{2-(2-trifluoromethylphenylthio)acetyl}spiro(indole-3,4′-piperidine)-2(1H)-one (20.0 mg, 0.0480 mmol) of example 70, a methylene chloride solution (2 mL) of m-chloroperbenzoic acid (8.30 mg, 0.0480 mmol) was added under ice-cold condition. The mixture was stirred at the same temperature for 1 hour. The reaction solution was added with water and extracted with chloroform. The organic layer was dried with anhydrous sodium sulfate, followed by a vacuum concentration. The resultant residue was purified by preparative thin-layer chromatography (hexane:ethyl acetate=1-3) and 1′-{2-(2-trifluoromethylphenylsulfinyl)acetyl}spiro(indole-3,4′-piperidine)-2(1H)-one (16.4 mg, 78.5%) was obtained as a white crystalline solid.

¹H-NMR (400 MHz, CDCl₃) δ; 1.89-2.03 (m, 4H), 3.71-3.94 (m, 4H), 4.05-4.10 (m, 1H), 4.32 (d, J=13.7 Hz, 1H), 6.92 (d, J=8.1 Hz, 1H), 7.07 (t, J=7.2 Hz, 1H), 7.22-7.27 (m, 2H), 7.67 (t, J=7.8 Hz, 1H), 7.77 (d, J=7.8 Hz, 1H), 7.85 (t, J=7.8 Hz, 1H), 8.20 (d, J=22.0 Hz, 1H), 8.36 (d, J=7.8 Hz, 1H).

IR(ATR); 3245,1705,1621,1471,1314,1117,1027,751 cm⁻¹

EI-MS m/z; 436(M⁺).

### EXAMPLE 75

### Production of 1′-{2-(2-trifluoromethylphenylsulfonyl)acetyl}spiro(indole-3,4′-piperidine)-2(1H)-one

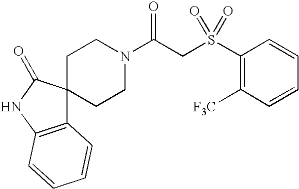

1′-{2-(2-ttrifluoromethylphenylsulfonyl)acetyl}spiro(i ndole-3,4-piperidin)-2(1H)-one was produced by the method described below.

To a methylene chloride solution (3 mL) of 1-{2-(2-trifluoromethylphenylthio)acetyl}spiro(indole-3,4′-piperidine)-2(1H)-one (20.0 mg, 0.0480 mmol) of example 70, a methylene chloride solution (2 mL) of m-chloroperbenzoic acid mg, 0.144 mmol) was added under ice-cold condition. The mixture was stirred at the same temperature for 1 hour. Then, the reaction solution was further added with a methylene chloride solution (2 mL) of m-chloroperbenzoic acid (15.3 mg, 0.0890 mmol) and stirred at room temperature for 40 minutes. The reaction solution was added with water and extracted with chloroform. The organic layer was dried with anhydrous sodium sulfate, followed by a vacuum concentration. The resultant residue was purified by preparative thin-layer chromatography (hexane:ethyl acetate=1:3) and 1′-{2-(2-trifluoromethylphenylsulfonyl)acetyl}spiro(indole-3,4′-piperidine)-2(1H)-one (12.0 mg, 55.3%) was obtained as a white crystalline solid.

¹H-NMR (400 MHz, CDCl₃) δ; 1.87-1.90 (m, 2H), 1.95-2.00 (m, 1H), 2.11-2.17 (m, 1H), 3.74-3.81 (m, 1H), 3.90-3.96 (m, 1H), 4.10-4.20 (m, 1H), 4.23-4.28 (m, 1H), 4.40 (d, J=14.0 Hz, 1H), 4.56 (d, J=14.0 Hz, 1H), 6.90(d, J=7.6 Hz, 1H), 7.08 (t, J=7.8 Hz, 1H), 7.21-7.25 (m, 3H), 7.81-7.83 (m, 2H), 7.94-7.96 (m, 1H), 8.32-8.34 (m, 1H).

IR(ATR); 1702,1637,1307,1159,748 cm⁻¹.

EI-MS m/z; 452(M⁺).

### EXAMPLE 76

### Production of 1-methyl-1′-{2-(2-trifluoromethylphenoxy)acetyl}spiro(indol e-3,4′-piperidine)-2-one

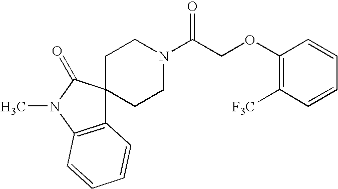

1-methyl-1′-{2-(2-trifluoromethylphenoxy)acetyl}spiro(indole-3,4′-piperidine)-2-one was produced by the method described below.

To a tetrahydrofuran solution (2 mL) of 1′-{2-(2-trifluoromethylphenoxy)acetyl}spiro(indole-3,4′-pi peridine)-2(1H)-one (15.0 mg, 0.0371 mmol) of example 1, a tetrahydrofuran solution (1 mL) of sodium hydride (4.00 mg, 0.111 mmol) was added under ice-cold condition. The mixture was stirred at the same temperature for 10 minutes, then added with excessive amounts of methyl iodide and stirred for 3.5 hours at room temperature. The reaction solution was added with water and extracted with diethylether. The organic layer was dried with anhydrous sodium sulfate, followed by a vacuum concentration. The resultant residue was purified by preparative thin-layer chromatography (hexane:ethyl acetate=1:1) and 1-methyl-1′-{2-(2-trifluoromethylphenoxy)acetyl}spiro(indol e-3,4′-piperidine)-2-one (18.0 mg, 100%) was obtained as colorless oil.

¹H-NMR (400 MHz, CDCl₃) δ; 1.78-1.82 (m, 4H), 3.20 (s, 3H), 3.80-3.87 (m, 1H), 3.95-4.00 (m, 1H), 4.08-4.14 (m, 1H), 4.23-4.28 (m, 1H), 4.82 (d, J=13.4 Hz, 1H), 4.92 (d, J=13.4 Hz, 1H), 6.85 (d, J=7.8 Hz, 1H), 7.05-7.07 (m, 2H), 7.09 (t, J=13.4 Hz, 1H), 7.18 (d, J=8.3 Hz, 1H), 7.26-7.31 (m, 1H), 7.54 (t, J=7.6 Hz, 1H), 7.61 (d, J=7.6 Hz, 1H).

IR(ATR); 3469,2936,1638,1237,1138,772 cm⁻¹.

EI-MS m/z; 418(M⁺).

### EXAMPLE 77

### Production of 1-benzyl-1′-{2-(2-trifluoromethylphenoxy)acetyl}spiro(indol e-3,4′-piperidine)-2-one

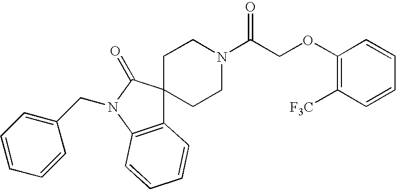

The reaction and treatment were conducted in a similar manner to the process of example 76 using benzyl bromide in place of methyl iodide, and the title compound was obtained as colorless oil.

¹H-NMR (400 MHz, CDCl₃) δ; 1.83-1.87 (m, 4H), 3.82-3.89 (m, 1H), 4.00-4.18 (m, 1H), 4.12-4.18 (m, 1H), 4.29-4.33 (m, 1H), 4.83 (d, J=13.4 Hz, 1H), 4.89 (s, 2H), 4.94 (d, J=13.4 Hz, 1H), 6.72 (d, J=7.6 Hz, 1H), 6.99-7.34 (m, 10H), 7.55(t, J=7.3 Hz, 1H), 7.61 (d, J=7.8 Hz, 1H).

IR(ATR); 2925,1700,1648,1609,1465,1321,1117,1038,757 cm⁻¹

EI-MS m/z; 494(M⁺).

### EXAMPLE 78

### Production of 1-isopropyl-1′-{2-(2-trifluoromethylphenoxy)acetyl}spiro(in dole-3,4′-piperidine)-2-one

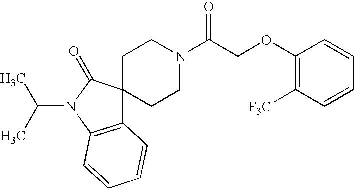

The reaction and treatment were conducted in a similar manner to the process of example 76 using 2-iodopropane in place of methyl iodide, and the title compound was obtained as colorless oil.

¹H-NMR (400 MHz, CDCl₃) δ; 1.47(d, J=7.1 Hz, 6H), 1.75-1.79 (m, 4H), 3.76-3.83 (m, 1H), 3.95-4.00 (m, 1H), 4.06-4.13 (m, 1H), 4.26-4.32 (m, 1H), 4.64 (quint, J=7.1 Hz, 1H), 4.82 (d, J=13.4 Hz, 1H), 4.93 (d, J=13.4 Hz, 1H), 7.00-7.03 (m, 3H), 7.09 (t, J=7.6 Hz, 1H), 7.19 (d, J=8.3H z, 1H), 7.22-7.26 (m, 1H), 7.54 (t, J=7.6 Hz, 1H), 7.61(d, J=7.8 Hz, 1H)

IR(ATR); 1697,1648,1608,1461,1321,1130,1038,750 cm⁻¹.

EI-MS m/z; 446(M⁺).

### EXAMPLE 79

### Production of 1-allyl-1′-{2-(2-trifluoromethylphenoxy)acetyl}spiro(indole-3,4′-piperidine)-2-one

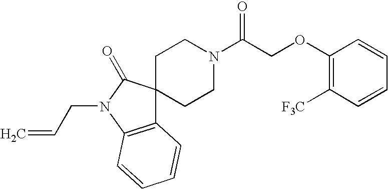

The reaction and treatment were conducted in a similar manner to the process of example 76 using allyl iodide in place of methyl iodide, and the title compound was obtained as a white amorphous solid.

¹H-NMR (400 MHz, CDCl₃) δ; 1.80-1.82 (m, 4H), 3.7.8-3.85 (m, 1H), 3.97-4.00 (m, 1H), 4.07-4.14 (m, 1H), 4.26-4.33 (m, 3H), 4.82 (d, J=13.4 Hz, 1H), 4.92 (d, J=13.4 Hz, 1H), 5.16-5.23 (m, 2H), 5.79-5.86 (m, 1H), 6.83 (d, J=7.8 Hz, 1H), 7.02-7.11 (m, 3H), 7.18 (d, J=8.5 Hz, 1H), 7.23-7.27(m, 1H), 7.54 (t, J=7.8 Hz, 1H), 7.61 (d, J=7.8 Hz, 1H).

IR(ATR); 1701,1648,1610,1466,1321,1118,1037,758 cm⁻¹.

EI-MS m/z; 444(M⁺).

### EXAMPLE 80

### Production of 1-ethyl-1′-{2-(2-trifluoromethylphenoxy)acetyl}spiro(indole-3,4′-piperidine)-2-one

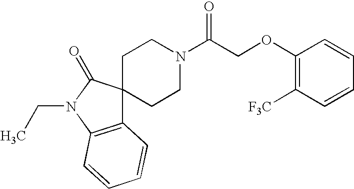

The reaction and treatment were conducted in a similar manner to the process of example 76 using ethyl iodide in place of methyl iodide, and the title compound was obtained as colorless oil.

¹H-NMR (400 MHz, CDCl₃) δ; 1.26(t, J=7.1 Hz, 3H), 1.78-1.81 (m, 4H), 3.74(q, J=7.1 Hz, 2H), 3.82 (quint, J=7.0 Hz, 1H), 3.95-4.01 (m, 1H), 4.07-4.14 (m, 1H), 4.23-4.29 (m, 1H), 4.83 (d, J=13.4 Hz, 1H), 4.93 (d, J=13.4 Hz, 1H), 6.86 (d, J=7.8 Hz, 1H), 7.02-7.06 (m, 2H), 7.09 (t, J=7.6 Hz, 1H), 7.18 (d, J=8.5 Hz, 1H), 7.26-7.30 (m, 1H), 7.54 (t, J=7.8 Hz, 1H), 7.61 (d, J=7.8 Hz, 1H).

EI-MS m/z; 432(M⁺).

### EXAMPLE 81

### Production of 1-propyl-1′-{2-(2-trifluoromethylphenoxy)acetyl}spiro(indol e-3,4′-piperidine)-2-one

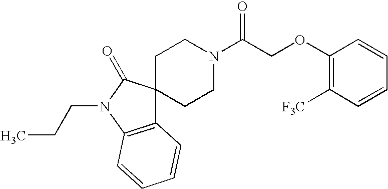

The reaction and treatment were conducted in a similar manner to the process of example 76 using propyl iodide in place of methyl iodide, and the title compound was obtained as a white amorphous solid.

¹H-NMR (400 MHz, CDCl₃) δ; 0.95(t, J=7.3 Hz, 3H), 1.70 (sext, J=7.3 Hz, 2H), 1.77-1.81 (m, 4H), 3.65 (t, J=7.3 Hz, 2H), 3.83 (quint, J=7.0 Hz, 1H), 3.98 (quint, J=7.0 Hz, 1H), 4.07-4.14 (m, 1H), 4.24-4.29 (m, 1 H), 4.82 (d, J=13.4 Hz, 1H), 4.93 (d, J=13.4 Hz, 1H), 6.85 (d, J=7.8 Hz, 1 H), 7.01-7.06 (m, 2H), 7.09 (t, J=7.8 Hz, 1H), 7.18 (d, J=8.3 Hz, 1H), 7.24-7.29 (m, 1H), 7.54 (t, J=7.8 Hz, 1H), 7.60 (d, J=7.8 Hz, 1H).

IR(ATR); 1700,1650,1610,1466,1322,1236,1130,1061,757 cm⁻¹ EI-MS m/z; 446(M⁺).

### EXAMPLE 82

### Production of 1-cyclopropylmethyl-1′-{2-(2-trifluoromethylphenoxy)acetyl}spiro(indole-3,4′-piperidine)-2-one

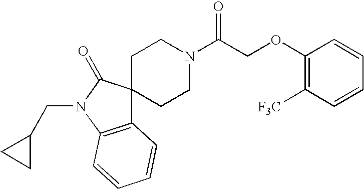

The reaction and treatment were conducted in a similar manner to the process of example 76 using cyclopropane methylbromide in place of methyl iodide, and the title compound was obtained as a white amorphous solid.

¹H-NMR (400 MHz, CDCl₃) δ; 0.34-0.55 (m, 4H), 1.10-1.20 (m, 1H) 1.80-1.82 (m, 4H), 3.58 (d, J=7.0 Hz, 2H), 3.82 (quint, J=7.0 Hz, 1H), 3.96-4.16 (m, 2H), 4.23-4.32 (m, 1H), 4.82 (d, J=13.2 Hz, 1H), 4.94 (d, J=13.2 Hz, 1H), 6.89 (d, J=7.8 Hz, 1H), 6.94-7.11 (m, 3H), 7.18 (d, J=8.0H z, 1H), 7.25-7.31 (m, 1H), 7.54 (t, J=8.0 Hz, 1H), 7.61 (d, J=8.0 Hz, 1H)

IR(ATR); 1700,1649,1610,1466,1321,1238,1118,1038,758 cm⁻¹

EI-MS m/z; 458(M⁺).

### EXAMPLE 83

### Production of 1-(4-pyridyl)methyl-1′-{2-(2-trifluoromethylphenoxy)acetyl}spiro(indole-3,4′-piperidine)-2-one

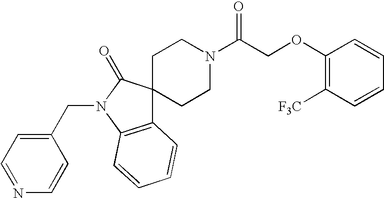

The reaction and treatment were conducted in a similar manner to the process of example 76 using 4-(methylbromide)pyridine bromate in place of methyl iodide, and the title compound was obtained as a white amorphous solid.

¹H-NMR (400 MHz, CDCl₃) δ; 1.86-1.88 (m, 4H), 3.84 (quint, J=7.3Hz, 1H), 4.02-4.16 (m, 2H, 4.30-4.36 (m, 1H), 4.84 (d, J=13.2 Hz, 1H), 4.90 (s, 2H), 4.94 (d, J=13.2 Hz, 1H), 6.64 (d, J=7.6 Hz, 1H), 7.04-7.20 (m, 7H), 7.55 (t, J=7.8 Hz, 1H), 7.62 (d, J=7.8 Hz, 1H), 8.56 (dd, J=4.5, 1.7 Hz, 2H).

IR(ATR); 1702,1651,1609,1466,1322,1237,1118,1038,759 cm⁻¹

EI-MS m/z; 495(M⁺).

### EXAMPLE 84

### Production of 1-cyclopropyl-1′-{2-(2-trifluoromethylphenoxy)acetyl}spiro(indole-3,4′-piperidine)-2-one

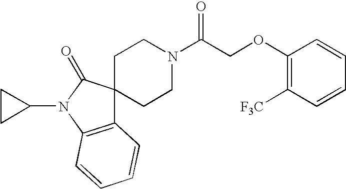

1-cyclopropyl-1′-{2-(2-trifluoromethylphenoxy)acetyl}spiro(indole-3,4′-piperidine)-2-one was produced by the method described below.

To a methylene chloride solution (2 mL) of 1′-{2-(2-trifluoromethylphenoxy)acetyl}spiro(indole-3,4′-pi peridine)-2(1H)-one (74.0 mg, 0.183 mmol) of example 1, copper acetate (II) (49.9 mg, 0.275 mmol), pyridine (43.4 mg, 0.549 mmol), and cyclopropylbismuth (III) (152 mg, 0.457 mmol) was added sequentially under an argon atmosphere at room temperature. The mixture was stirred at 50° C. for 14 hours. The reaction solution was added with water and extracted with chloroform. The organic layer was dried with anhydrous sodium sulfate, followed by a vacuum concentration. The resultant residue was purified by preparative thin-layer chromatography (hexane:ethyl acetate=1:1) and 1-cyclopropyl-1′-{2-(2-trifluoromethylphenoxy)acetyl}spiro(indole-3,4′-piperidine)-2-one (48.8 mg, 60.0%) was obtained as colorless oil.

¹H-NMR (400 MHz, CDCl₃) δ; 0.86-0.90 (m, 2H), 1.04-1.10(m, 2H), 1.74-1.79 (m, 4H), 2.60-2.66 (m, 1H), 3.80 (quint, J=7.3 Hz, 1H), 3.93-3.99 (m, 1H), 4.05-4.15 (m, 1H), 4.22-4.28 (m, 1H), 4.82 (d, J=13.5 Hz, 1H), 4.92 (d, J=13.5 Hz, 1H), 7.01-7.13 (m, 4H), 7.17 (d, J=8.3 Hz, 1H), 7.26-7.32 (m, 1H), 7.53 (t, J=8.3 Hz, 1H), 7.60 (d, J=7.8 Hz, 1H).

IR(ATR); 3469,2936,1638,1237,1138,772 cm⁻¹.

EI-MS m/z; 444(M⁺).

### EXAMPLE 85

### Production of 1′-{4-(2-trifluoromethylphenoxy)butanoyl}spiro(indole-2,4′-pip eridine)-2(1H)-one

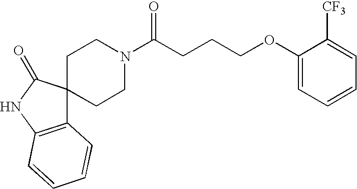

The reaction and treatment were conducted in a similar manner to process 3 of example 1 using 4-(2-trifluoromethylphenoxy)butanoic acid in place of 2-trifluoromethylphenoxyacetic acid., and the title compound was obtained as a white amorphous solid.

¹H-NMR (400 MHz, CDCl₃) δ; 1.76-1.96 (m, 4H), 2.18-2.26 (m, 2H), 2.63-2.70 (m, 2H), 3.75-3.86 (m, 2H), 3.96-4.04 (m, 1H), 4.11-4.25 (m, 3H), 6.89 (d, J=7.6 Hz, 1H), 7.02-7.03 (m, 3H), 7.17 (d, J=7.3 Hz, 1H), 7.23 (t, J=7.6 Hz, 1H), 7.50 (t, J=8.2 Hz, 1H), 7.55 (d, J=7.8 Hz, 1H), 7.67 (s, 1H).

IR(ATR); 3204,2941,1706,1609,1497,1472,1460,1323,1275,1 257,1165,1115,1057,1037,946,844,753,701 cm⁻¹.

EI-MS m/z; 432(M⁺).

### EXAMPLE 86

### Production of 1′-{2-(2,4-bistrifluoromethylphenoxy)acetyl}spiro(indole-2,4′-piperidine)-2(1H)-one

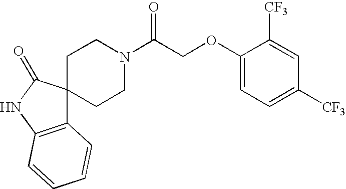

The reaction and treatment were conducted in a similar manner to process 2 of example 2 using 2,4-bistrifluoromethylphenol in place of ortho-cresol, and the title compound was obtained as a white amorphous solid.

¹H-NMR (400 MHz, CDCl₃) δ; 1.73-1.94(m, 4H), 3.70-3.80(m, 1H), 3.86-3.92 (m, 1H), 4.01-4.14 (m, 1H), 4.17-4.23 (m, 1H), 4.91 (d, J=13.6 Hz, 1H), 5.00(d, J=13.6 Hz, 1H), 6.90 (d, J=7.8 Hz, 1H), 7.02-7.08 (m, 2H), 7.23-7.24 (m, 1H), 7.30 (d, J=8.8 Hz, 1H), 7.80 (d, J=8.2 Hz, 1H), 7.88 (s, 1H), 8.28(s, 1H).

IR(ATR); 3195,1706,1666,1654,1622,1472,1348,1283,1265,1 127,1068,917,747 cm⁻¹.

EI-MS m/z; 472(M⁺).

### EXAMPLE 87

### Production of 2-oxo-N-{2-(trifluoromethyl)benzyl}spiro(indoline-3,4′-pipe ridine)-1′-carboxamide

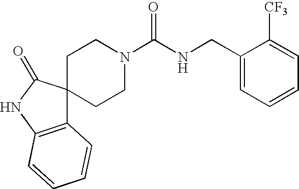

2-oxo-N-{2-(trifluoromethyl)benzyl}spiro(indoline-3,4′-piperidine)-1′-carboxamide was produced by the method described below.

To an acetonitrile solution (3 mL) of spiro(indole-3,4′-piperidine)-2(1H)-one (20.2 mg, 0.10 mmol), 4-nitrophenylchloroformate (20.1 mg, 0.10 mmol), diisopropylethylamine (26.0 mg, 0.20 mmol), and 2-trifluoromethylbenzylamine (17.5 mg, 0.10 mmol) were added at room temperature. The mixture was stirred at the same temperature for 2 hours. The reaction solution was added with water and extracted with chloroform. The organic layer was washed with brine and dried with anhydrous sodium sulfate, followed by a vacuum concentration. The resultant residue was purified by preparative thin-layer chromatography (hexane:ethyl acetate=3:1), and 2-oxo-N-{2-(trifluoromethyl)benzyl}spiro(indoline-3,4′-pipe ridine)-1′-carboxamide (10.4 mg, 25.8%) was obtained as white crystalline powder.

¹H-NMR (400 MHz, acetone-d₆) δ; 1.76-1.82 (m, 4H), 3.60-3.63 (m, 2H), 3.79-3.93 (m, 2H), 4.65 (d, J=5.8 Hz, 2H), 6.56 (s, 1H), 6.92-6.93 (m, 1H), 7.00-7.02 (m, 1H), 7.19-7.23 (m, 1H), 7.41-7.44 (m, 2H), 7.60-7.69(m, 3H), 9.40 (s, 1H).

IR(ATR); 2954,1707,1621,1537,1313,1118,767 cm⁻¹.

EI-MS m/z; 403(M⁺).

### EXAMPLE 88

### Production of N,1-dimethyl-2-oxo-N-{2-(trifluoromethyl)benzyl}spiro(indol ine-3,4′-piperidine)-1′-carboxamide

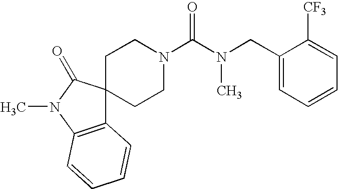

N,1-dimethyl-2-oxo-N-{2-(trifluoromethyl)benzyl}spiro (indoline-3,4′-piperidine)-1′-carboxamide was produced by the method described below.

To a tetrahydrofuran solution (1 mL) of 2-oxo-N-{2-(trifluoromethyl)benzyl}spiro(indoline-3,4′-pipe ridine)-1′-carboxamide (4.00 mg, 0.01 mmol), sodium hydride (4.80 mg, 0.10 mmol) and methyl iodide (14.1 mg, 0.10 mmol) was added at room temperatures. The mixture was stirred at the same temperature for 20 hours. The reaction solution was added with water and extracted with chloroform. The organic layer was washed with brine and dried with anhydrous sodium sulfate, followed by a vacuum concentration. The resultant residue was purified by preparative thin-layer chromatography (hexane:ethyl acetate=3:1) and N,1-dimethyl-2-oxo-N-{2-(trifluoromethyl)benzyl}spiro(indol ine-3,4′-piperidine)-1′-carboxamide (4.40 mg, 25.8%) was obtained as white crystalline powder.

¹H-NMR (400 MHz, CDCl₃) δ; 1.81-1.89 (m, 4H), 2.85 (s, 3H), 3.21 (s, 3H), 3.54-3.59(m, 2H), 3.77-3.83 (m, 2H), 4.65 (s, 2H), 6.85 (d, J=7.8 Hz, 1H), 7.07 (t, J=7.8 Hz, 1H), 7.25-7.28 (m, 3H), 7.51-7.55 (m, 2H), 7.66 (d, J=7.8 Hz, 1H).

### EXAMPLE 89

### Production of 1-{2-(2-trifluoromethylphenoxy)acetyl}spiro{indole-3,4′-(t rans-2′,6′-dimethyl)piperidine)-2(1H)-one

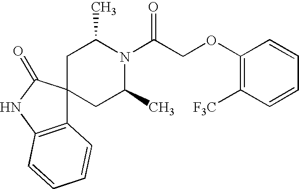

### Process 1

### Production of N-{2-(2′-trifluoromethylphenoxy)acetyl}-trans-2,6-dimethylppiperidine-4-one

N-{2-(2′-trifluoromethylphenoxy)acetyl}-trans-2,6-dim ethyl-piperidine-4-one was produced by the method described below.

To a methylene chloride solution (3 mL) of 2-trifluoromethylphenoxyacetic acid (760 mg, 3.45 mmol), oxalyl chloride (876 mg, 6.90 mmol) and N,N-dimethylformamide mL) was added at room temperature. The mixture was stirred at the same temperature for 1 hour. The reaction solution was subjected to a vacuum concentration and the residue was added with methylene chloride (5 mL) and dissolved. This solution was added with N-benzyl-trans-2,6-dimethylpiperidine-4-one (500 mg, 2.30 mmol), triethylamine (466 mg, 4.60 mmol) and stirred at room temperature for 2 hours. The reaction solution was added with water and extracted with chloroform. The organic layer was dried with anhydrous sodium sulfate, followed by a vacuum concentration. The resultant residue was purified by silica-gel chromatography (hexane:ethyl acetate=1:1) and N-{2-(2′-trifluoromethylphenoxy)acetyl}-trans-2,6-dimethylppiperidine-4-one (417 mg, 55.0%) was obtained as a pale yellow crystal.

¹H-NMR (270 MHz, CDCl₃) δ; 1.59(s, 6H), 2.45 (d, J=17.8 Hz, 2H), 2.86 (d, J=5.9 Hz, 1H), 2.92 (d, J=5.9 Hz, 1H), 4.65-4.80 (m, 2H), 4.86 (q, J=13.2 Hz, 2H), 7.11 (t, J=9.0 Hz, 2H), 7.50 (t, J=7.3 Hz, 1H), 7.60 (d, J=7.3 Hz, 1H).

### Process 2

### Production of N-{2-(2′-trifluoromethylphenoxy)acetyl}-trans-5,7-dimethyl-1-oxa-6-azaspiro[2,5]octane

N-{2-(2′-trifluoromethylphenoxy)acetyl}-trans-5,7-dim ethyl-1-oxa-6-azaspiro[2,5]octane was produced by the method described below.

To a dimethylsulfoxide solution (5 mL) of N-{2-(2′-trifluoromethylphenoxy)acetyl}-trans-2,6-dimethylppiperidine-4-one (415 mg, 1.26 mmol), sodium hydride (63.5 mg, 2.65 mmol) and trimethylsulfoxonium iodide (55.5 mg, 2.52 mmol), were added under an argon atmosphere at room temperature. The mixture was stirred at the same temperature for 12 hours. The reaction solution was added with water and extracted with diethylether. The organic layer was dried with anhydrous magnesium sulfate, followed by a vacuum concentration. The resultant residue was purified by silica-gel chromatography (hexane:acetone=3:1) and N-{2-(2′-trifluoromethylphenoxy)acetyl}-trans-5,7-dimethyl-1-oxa-6-azaspiro[2,5]octane (385 mg, 89.0%) was obtained as pale-yellow oil.

¹H-NMR (400 MHz, CDCl₃) δ; 1.26-1.74 (m, 8H), 2.37 (d, J=12.9 Hz, 1 H), 2.57 (dd, J=5.0, 15.5 Hz, 1H), 2.70 (q, J=5.0 Hz, 2H), 4.42 (s, 2H), 4.81 (q, J=13.6 Hz, 2H), 7.05 (t, J=7.8 Hz, 1H), 7.10 (d, J=8.6 Hz, 1H), 7.49 (t, J=7.8 Hz, 1H), 7.58 (d, J=7.1 Hz, 1H).

### Process 3

### Production of N-{2-(2′-trifluoromethylphenoxy)acetyl}-trans-2,6-dimethylppiperidine-4-carboxaldehyde

N-{2-(2′-trifluoromethylphenoxy)acetyl}-trans-2,6-dim ethyl-piperidine-4-carboxaldehyde was produced by the method described below.

To a methylene chloride solution (5 mL) of N-{2-(2′-trifluoromethylphenoxy)acetyl}-trans-5,7-dimethyl-1-oxa-6-azaspiro[2,5]octane (380 mg, 1.11 mmol), botontrifluoride diethylether (236 mg, 1.66 mmol) was added at room temperature. The mixture was stirred at the same temperature for 3 hours. The reaction solution was added with water and extracted with chloroform. The organic layer was dried with anhydrous sodium sulfate, followed by a vacuum concentration. The resultant residue was purified by preparative thin-layer chromatography (hexane:acetone=2:1) and N-{2-(2′-trifluoromethylphenoxy)acetyl}-trans-2,6-dimethylppiperidine-4-carboxaldehyde (277 mg, 72.9%) was obtained as pale-yellow oil.

¹H-NMR (400 MHz, CDCl₃) δ; 1.17(d, J=6.8 Hz, 3H), 1.31 (d, J=6.3 Hz, 3H), 1.93-2.00 (m, 1H), 2.13-2.21 (m, 3H), 2.76-2.82 (m, 1H), 4.21-4.26 (m, 1H), 4.44 (brs, 1H), 4.74 (d, J=13.6 Hz, 1H), 4.81 (d, J=13.6 Hz, 1H), 7.02-7.07 (m, 2H), 7.46 (t, J=7.3 Hz, 1H), 7.58 (d, J=7.8 Hz, 1H), 9.75 (s, 1H).

### Process 4

### Production of 1-{2-(2-methylphenoxy)acetyl}spiroindole-3′,4-piperidine

1-{2-(2-methylphenoxy)acetyl}spiroindole-3′,4-piperid ine was produced by the method described below.

To a mixed solution of trifluoroacetic acid, acetonitrile, and toluene (1:1:50) (5.2 mL) of N-{2-(2′-trifluoromethylphenoxy)acetyl}-trans-2,6-dimethylppiperidine-4-carboxaldehyde (275 mg, 0.800 mmol), phenylhydrazine (95.3 mg, 0.880 mmol) was added at room temperature. The mixture was stirred at 40° C. for 17 hours. The reaction solution was added with water and extracted with ethyl acetate. The organic layer was dried with anhydrous sodium sulfate, followed by a vacuum concentration. The resultant residue was purified by preparative thin-layer chromatography (hexane:ethyl acetate=1:2) and 1-{2-(2-methylphenoxy)acetyl}spiroindole-3′,4-piperidine mg, 53.4%) was obtained as a pale-yellow amorphous solid.

¹H-NMR (400 MHz, CDCl₃) δ; 1.53(d, J=6.8 Hz, 3H), 1.61 (d, J=6.8 Hz, 3H), 1.65-1.86 (m, 2H), 2.14 (brs, 1H), 2.44 (dd, J=5.2, 14.8 Hz, 1H), 4.32 (brs, 1H), 4.50-4.60 (m, 1H), 4.86 (s, 2H), 7.08 (t, J=7.7 Hz, 1H), 7.15 (d, J=8.3 Hz, 1H), 7.28 (t, J=6.2 Hz, 1H), 7.33-7.39 (m, 2H), 7.52 (t, J=7.4 Hz, 1H), 7.60-7.64 (m, 2H), 8.27 (s, 1H).

IR(ATR); 1654,1462,1321,1118,1038,754 cm⁻¹.

EI-MS m/z; 416(M⁺).

### Process 5

### Production of 1′-{2-(2-trifluoromethylphenoxy)acetyl}spiro{(indole-3,4′)-trans-2,6-dimethylpiperidine-2(1H)-one

1′-{2-(2-trifluoromethylphenoxy)acetyl}spiro{(indole-3,4′)-trans-2,6-dimethylpiperidine-2(1H)-one was produced by the method described below.

To a methylene chloride solution (5 mL) of 1-(2-(2-methylphenoxy)acetyl}spiroindole-3′,4-piperidine mg, 0.410 mmol), 3-chloroperbenzoic acid (106 mg, 0.610 mmol) was added at room temperature. The mixture was stirred at the same temperature for 5 hours. The reaction solution was added with water and extracted with chloroform. The organic layer was dried with anhydrous sodium sulfate, followed by a vacuum concentration. The resultant residue was purified by preparative thin-layer chromatography (hexane:acetone=1:2) and 1′-{2-(2-trifluoromethylphenoxy)acetyl}spiro{(indole-3,4′)-trans-2,6-dimethylpiperidine}-2(1H)-one (84.0 mg, 47.6%) was obtained as a pale-yellow amorphous solid.

¹H-NMR (400 MHz, CDCl₃) δ; 1.35(dd, J=6.7, 13.8 Hz, 3H), 1.56 (t, J=6.7 Hz, 3H), 2.40-2.46 (m, 1H), 2.66-3.13 (m, 3H), 4.23-4.66 (m, 2H), 4.76 (s, 2H), 7.03-7.58 (m, 5H), 7.82-7.94 (m, 3H), 8.48 (d, J=11.0 Hz, 1H).

IR(ATR); 1687,1654,1322,1249,1117,750 cm⁻¹.

EI-MS m/z; 432(M⁺).

### TEST EXAMPLE 1

### Human HSD1-, HSD2-Iinhibitory Effect

Human 11β-HSD1-, human 11β-HSD2-Gene Clonings and Establishment of Stably Expressing Cells

Human 11β-HSD1- and human 11β-HSD2-gene clonings were conducted using as a template a reverse transcription product of human-liver RNA and human-kidney RNA (CELL APPLICATIONS) respectively, by means of PCR cloning with reference to nucleotide sequences of Genbank Accession No. NM_{—}00.5525 and NM 000196. The obtained PCR products of about 0.9 kbp and 1.2 kbp were subcloned into an expression vector pcDNA3.1+/Zeo (Invitrogen).

Human 11β-HSD1- and human 11β-HSD2-expressing vectors were transfected into human kidney-derived cell line, HEK293 cells, using a transfection reagent, jet PEI (Funakoshi). Selection was conducted with 400 μg/mL of zeocine (Invitrogen) to provide stably-expressing-cell clones. The stably expressing cells were suspended in buffer solution A (20 mmol/L Tris-HCl, pH 7.4, 250 mmol/L sucrose, 1 mmol/L EGTA, 1 mmol/L EDTA, 1 mmol/L MgCl₂), sonicated, and then stored at −80° C.

### Assay of Enzyme Inhibitory Activity

An enzymatic reaction was conducted using a polystyrene 96-well plate. Each well was added with 1 μL of a test agent dissolved in DMSO and then diluted (0.003 to 3 mmol/L), and further added with 10 μL of cell lysate diluted to a concentration of 0.1 mg/mL to 0.4 mg/mL. Next, 90 μL of buffer solution A containing substrate (100 nmol/L cortisone) and coenzyme (400 μmol/L NADPH) was added and the mixture was incubated at 37° C. for 1 hour. The enzymatic reaction was stopped by treating at 95° C. for 3 minutes. Cortisol that was present in the reaction solution was determined by a competitive ELISA shown below.

Anti-rabbit IgG antibody (Chemi-con) diluted to 2 μg/mL with carbonate buffer solution (pH 9.6) was added in 100 μL each to a 96-well immuno plate (Nunc) and immobilized by incubating at 4° C. overnight. 50 μL of enzymatic reaction solution was put onto the plates, and further, anti-cortisol antibody (Cosmo Bio) and HRP-labeled cortisol (Cosmo Bio), diluted with buffer solution B (25 mmol/L Tris-HCl pH 7.4, 137 mmol/L NaCl, 2.68 mmol/L KCl), were added in 50 μL respectively and incubated at 4° C. overnight. After washed three times with buffer solution B containing 0.05% Tween 20, the plates were allowed to develop color by adding 100 μL of color reagent, TMB (Moss). The color reaction was stopped by 25 μL of 1 mol/L sulfuric acid and the absorbance was determined at 450 nm with a microplate reader (Molecular Device, VersaMax).

The values of human 11β-HSD1 and human 11β-HSD2 activities were subtracted from 100, and the resultant values were regarded as the respective 11β-HSD inhibition rates of example compounds. For each example compound, the value of 50% inhibitory concentration (IC₅₀) was calculated from 11β-HSD inhibition rates at plural concentrations, for 11β-HSD1 and 11β-HSD2 activities. The results are shown in table 1.

TABLE 1

| | |
|---|---|
| Example | IC₅₀(μM) |

| No. | HSD1 | HSD2 |
|---|---|---|
| 1 | 0.033 | 30 |
| 2 | 0.82 | NA |
| 10 | 0.087 | >30 |
| 12 | 1.00 | NA |
| 40 | 0.96 | 30 |
| 49 | 0.41 | >30 |
| 52 | 0.73 | NA |
| 70 | 0.16 | 30 |
| 76 | 0.068 | >30 |
| 80 | 0.67 | 30 |
| 81 | 0.43 | NA |
| 84 | 0.72 | NA |

Surprisingly, actually synthesizing example 56 (compound A) of patent document 1 and measuring the inhibition rate revealed that the 50% inhibitory concentration (IC₅₀) of compound A was 3.0 μM for 11β-HSD1 activity, which represented a great difference from the present invention in activity.

1. A spiro oxindole compound represented by the following general formula (1) or salt thereof, or their solvate: 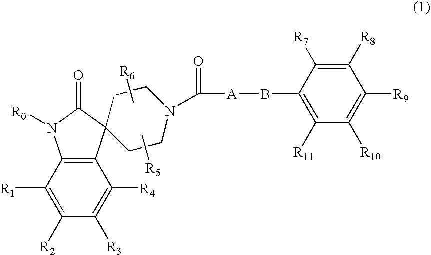
(wherein A is a —CR₁₂R₁₃—(CH₂)ₙ— or —NR₁₄—, B is an oxygen atom, sulfur atom, —NR₁₅—, —CR₁₆R₁₇—, sulfonyl group, sulfinyl group, or —NHCO—CH₂— (here, when A is —NR₁₄—, B is —CR₁₆R₁₇—); R₀ is a hydrogen atom, alkyl group with 1 to 6 carbon atoms, alkenyl group with 2 to 6 carbon atoms, (aryl with 6 to 14 carbon atoms)-alkyl group with 1 to 6 carbon atoms, cycloalkyl group with 3 to 6 carbon atoms, (cycloalkyl with 3 to 6 carbon atoms)-alkyl group with 1 to 6 carbon atoms, or (5 to 14-membered heteroaryl)-alkyl group with 1 to 6 carbon atoms; each R₁, R₂, R₃, and R₄ are same or different and are a hydrogen atom, halogen atom, alkyl group with 1 to 6 carbon atoms, haloalkyl group with 1 to 6 carbon atoms, alkenyl group with 2 to 6 carbon atoms, (alkoxy with 1 to 6 carbon atoms)-alkyl group with 1 to 6 carbon atoms, alkynyl group with 2 to 6 carbon atoms, aryl group with 6 to 14 carbon atoms, aryloxy group with 6 to 14 carbon atoms, cycloalkyl group with 3 to 6 carbon atoms, 5 to 14-membered heteroaryl group, 5 to 14-membered heteroaryloxy group, or 5 to 7-membered heterocycloalkyl group; each R₅ and R₆ are same or different and are a hydrogen atom, alkyl group with 1 to 6 carbon atoms, haloalkyl group with 1 to 6 carbon atoms, alkenyl-group with 2 to 6 carbon atoms, (alkoxy with 1 to 6 carbon atoms)-alkyl group with 1 to 6 carbon atoms, alkynyl group with 2 to 6 carbon atoms, aryl group with 6 to 14 carbon atoms, aryloxy group with 6 to 14 carbon atoms, cycloalkyl group with 3 to 6 carbon atoms, 5 to 14-membered heteroaryl group, 5 to 14-membered heteroaryloxy group, or 5 to 7-membered heterocycloalkyl group;
each R₇, R₈, R₉, R₁₀, and R₁₁ are same or different and are a hydrogen atom, halogen atom, amino group, cyano group, nitro group, hydroxy group, alkoxy group with 1 to 6 carbon atoms, haloalkoxy group with 1 to 6 carbon atoms, mono- or di-alkylamino group with 1 to 6 carbon atoms, alkyl group with 1 to 6 carbon atoms, haloalkyl group with 1 to 6 carbon atoms, alkenyl group with 2 to 6 carbon atoms, (alkoxy with 1 to 6 carbon atoms)-alkyl group with 1 to 6 carbon atoms, alkynyl group with 2 to 6 carbon atoms, alkylsulfinyl group with 1 to 6 carbon atoms, alkylsulfonyl group with 1 to 6 carbon atoms, aryl group with 6 to 14 carbon atoms, aryloxy group with 6 to 14 carbon atoms, cycloalkyl group with 3 to 6 carbon atoms, 5 to 14-membered heteroaryl group, 5 to 14-membered heteroaryloxy group, or 5 to 7-membered heterocycloalkyl group, or R₁₇ and R₈, or R₈ and R₉ may together form a benzene ring;
when they exist together, each R₁₂, R₁₃, R₁₄, R₁₅, R₁₆, and R₁₇, same or different, are a hydrogen atom, alkyl group with 1 to 6 carbon atoms, haloalkyl group with 1 to 6 carbon atoms, or alkenyl group with 2 to 6 carbon atoms; and
n is an integer of 0 to 10).

2. A spiro oxindole compound or salt thereof, or their solvate according to claim 1, wherein the compound represented by general formula (1) is:
1′-{2-(2-trifluoromethylphenoxy)acetyl}spiro(indole-3,4′-pi peridine)-2(1H)-one,
1′-{2-(2-methylphenoxy)acetyl}spiro(indole-3,4′-piperidine)-2(1H)-one,
1′-{2-(2-iodophenoxy)acetyl}spiro(indole-3,4′-piperidine)-2 (1H)-one,
1′-{2-(2-nitrophenoxy)acetyl}spiro(indole-3,4′-piperidine)-2(1H)-one,
1′-{2-(2,3-dichlorophenoxy)acetyl}spiro(indole-3,4′-piperid ine)-2(1H)-one,
1′-{2-(2-bromophenoxy)acetyl}spiro(indole-3,4′-piperidine)-2(1H)-one,
1′-{2-(2-t-butylphenoxy)acetyl}spiro(indole-3,4′-piperidine)-2(1H)-one,
1′-{2-(2-trifluoromethylphenylthio)acetyl}spiro(indole-3,4′-piperidine)-2(1H)-one,
1-methyl-1′-{2-(2-trifluoromethylphenoxy)acetyl}spiro(indol e-3,4′-piperidine)-2-one,
1-ethyl-1′-{2-(2-trifluoromethylphenoxy)acetyl}spiro(indole-3,4′-piperidine)-2-one,
1-propyl-1′-{2-(2-trifluoromethylphenoxy)acetyl}spiro(indol e-3,4′-piperidine)-2-one, or
1-cyclopropyl-1′-{2-(2-trifluoromethylphenoxy)acetyl}spiro(indole-3,4′-piperidine)-2-one.

3. A pharmaceutical composition consisting of the spiro oxindole compound or salt thereof, or their solvate according to claim 1 or 2 and a pharmaceutically acceptable carrier.

4. An inhibitor of 11β-hydroxysteroid dehydrogenasel, which inhibitor comprises the spiro oxindole compound or salt thereof, or their solvate according to claim 1 or 2 as an active ingredient.

5. An agent for preventing and/or treating diabetes, insulin resistance, diabetes complication, obesity, dyslipidemia, hypertension, fatty liver, or metabolic syndrome, which agent comprises the spiro oxindole compound or salt thereof, or their solvate according to claim 1 or 2 as an active ingredient.

6. Use of the spiro oxindole compound or salt thereof, or their solvate according to claim 1 or 2 for producing a formulation for inhibiting 11β-hydroxysteroid dehydrogenasel.

7. Use of the spiro oxindole compound or salt thereof, or their solvate according to claim 1 or 2 for producing a formulation for an agent for preventing and/or treating diabetes, insulin resistance, diabetes complication, obesity, dyslipidemia, hypertension, fatty liver, or metabolic syndrome.

8. A method for inhibiting 11β-hydroxysteroid dehydrogenasel, which method comprises administering an effective amount of the spiro oxindole compound or salt thereof, or their solvate according to claim 1 or 2.

9. A method for preventing and/or treating diabetes, insulin resistance, diabetes complication, obesity, dyslipidemia, hypertension, fatty liver, or metabolic syndrome, which method comprises administering an effective amount of the spiro oxindole compound or salt thereof, or their solvate according to claim 1 or 2.
